(19) United States
(12) Patent Application Publication
(10) Pub. No.: US 2004/0147612 A1
(43) Pub. Date: Jul. 29, 2004

(54) Sulfonamide derivatives

(76) Inventors: Joshua Zwick Davison, Indianapolis, IN (US); Eric George Tromiczak, Indianapolis, IN (US); Hamideh Zarrinmayeh, Carmel, IN (US); Dennis Michael Zimmerman, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/479,283

(22) PCT Filed: May 23, 2002
(86) PCT No.: PCT/US02/15139

(51) Int. Cl. A61K 31/18

(52) U.S. Cl. 514/602; 564/79; 564/80

(57) ABSTRACT

The present invention provides certain sulfonamide derivatives of formula (I) (in which the variables are as defined in the claims) useful for potentiating glutamate receptor function in a patient and therefore useful for treating a wide variety of conditions, such as psychiatric and neurological disorders.

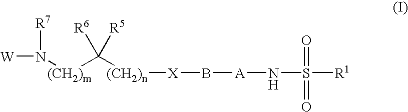

The present invention relates to the potentiation of glutamate receptor function using certain sulfonamide derivatives. It also relates to novel sulfonamide derivatives, to processes for their preparation and to pharmaceutical compositions containing them.

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these sub-units are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1 B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647. The physiological implications of rapid desensitization, and deactivation if any, are not fully understood.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

AMPA receptor potentiators have been shown to improve memory in a variety of animal tests. Staubli et al., Proc. Natl. Acad. Sci., Vol. 91, pp 777-781, 1994, Neurobiology, and Arai et al., The Journal of Pharmacology and Experimental Therapeutics, 278: 627-638, 1996.

In addition, certain sulfonamide derivatives which potentiate glutamate receptor function in a mammal have been disclosed in the following International Patent Application Publications: WO 98/33496 published Aug. 6, 1998; WO 99/43285 published Sep. 2, 1999; WO 00/06539; WO 00/06537, WO 00/06176, WO 00/06159, WO 00/06158, WO 00/06157, WO 00/06156, WO 00/06149, WO 00/06148, and WO 00/06083, all published Feb. 10, 2000; and WO 00/66546 published Nov. 9, 2000.

The present invention provides compounds of formula I:

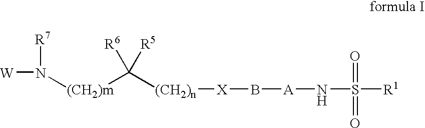

wherein

A represents

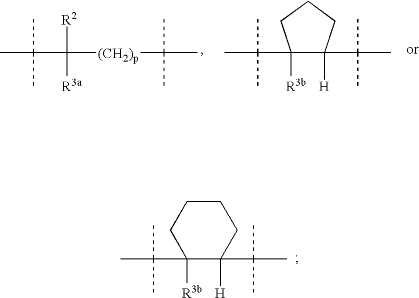

B represents

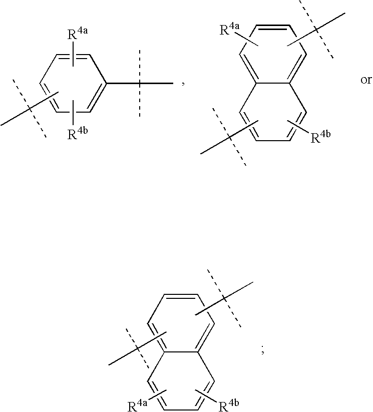

X represents O, NR, or S:

W represents R⁸SO₂—, R¹³C(═O)—, R¹³R¹⁵NC(═O)—, H₂NC(═O)—, R¹⁶, or R¹⁴OC(═O)—;

R represents hydrogen, (1-6C)alkyl, or -(1-4C)alkylaromatic;

R¹ represents (1-6C)alkyl, (2-6C)alkenyl, halo(1-4C)alkyl, or NR⁹R¹⁰;

R² and R^{3a} each independently represent hydrogen, (1-4C)alkyl, F, or —OR¹¹;

R^{3b} represents hydrogen, (1-4C)alkyl, or —OR¹²;

R^{4a} and R^{4b} each independently represent hydrogen, (1-4C) alkyl, (1-4C)alkoxy, I, Br, Cl, or F;

R⁵ and R⁶ each independently represent hydrogen, (1-4C)alkyl, F, or —OR¹¹;

R⁷ represents hydrogen, (1-4C)alkyl or —(1-4C)alkylaromatic;

R⁸ represents (1-6C)alkyl,-(1-4C)alkylphenyl, halo(1-4C)alkyl, unsubstituted or substituted aromatic group, unsubstituted or substituted heteroaromatic group, cycloalkyl, alkylcycloalkyl or NR⁹R¹⁰;

n is zero or an integer 1, 2, 3, 4, or 5;

m is zero or an integer 1, 2, 3, 4, or 5;

p is an integer 1 or 2;

R⁹ and R¹⁰ each independently represent hydrogen or (1-4C)alkyl;

R¹¹ represents hydrogen or (1-4C)alkyl;

R¹² represents (1-4C)alkyl;

R¹³ represents phenyl or (1-6C)alkyl;

R¹⁴ represents (1-6C)alkyl;

R¹⁵ represents (1-4C)alkyl; and

R¹⁶ represents (1-4C)alkyl or -(1-4C)alkylphenyl;

or a pharmaceutically acceptable salt thereof, with the proviso that when W is R¹⁶, then B is other than

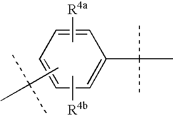

The present invention further provides a method of potentiating glutamate receptor function in a patient, which comprises administering to said patient an effective amount of a compound of formula I.

The present invention provides a method of treating cognitive disorders in a patient, which comprises administering to said patient an effective amount of a compound of formula I.

The present invention provides a method of treating depression in a patient, which comprises administering to said patient an effective amount of a compound of formula I.

The present invention provides a method of treating Alzheimer's disease in a patient, which comprises administering to said patient an effective amount of a compound of formula I.

In addition, the present invention further provides a method of treating psychosis or cognitive deficits associated with psychosis in a patient, which comprises administering to said patient an effective amount of a compound of formula I.

According to another aspect, the present invention provides the use of a compound of formula I, or a pharmaceutically acceptable salt thereof for the manufacture of a medicament for potentiating glutamate receptor function.

In addition, the present invention provides the use of a compound of formula I or a pharmaceutically acceptable salt thereof for potentiating glutamate receptor function.

The invention further provides pharmaceutical compositions comprising, a compound of formula I and a pharmaceutically acceptable diluent or carrier.

This invention also encompasses novel intermediates and processes for the synthesis of the compounds of formula I.

In addition, the present invention includes compounds of the formula:

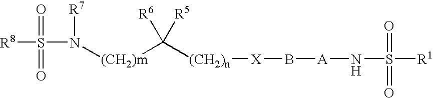

wherein

A represents

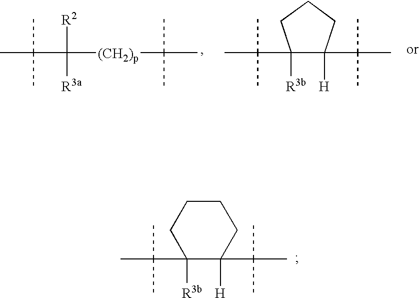

B represents

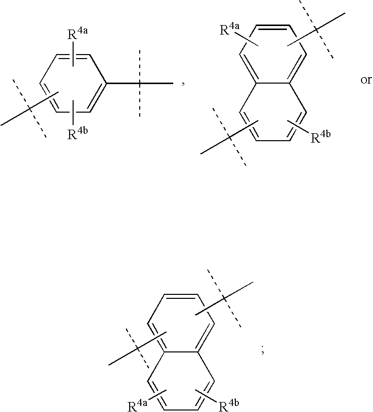

X represents O, NR, or S:

R represents hydrogen, (1 -6C)alkyl, or -(1-4C)alkylaromatic;

R¹ represents (1-6C)alkyl, (2-6C)alkenyl, halo(1-4C)alkyl, or NR⁹R¹⁰;

R² and R^{3a} each independently represent hydrogen, (1-4C)alkyl, F, or —OR¹¹;

R^{3b} represents hydrogen, (1-4C)alkyl, or —OR¹²;

R^{4a} and R^{4b} each independently represent hydrogen, (1-4C) alkyl, (1-4C)alkoxy, I, Br, Cl, or F;

R⁵ and R⁶ each independently represent hydrogen, (1-4C)alkyl, F, or —OR¹¹;

R⁷ represents hydrogen;

R⁸ represents (1-6C)alkyl;

n is zero or an integer 1, 2, 3, 4, or 5;

m is zero or an integer 1, 2, 3, 4, or 5;

p is an integer 1 or 2;

R⁹ and R¹⁰ each independently represent hydrogen or (1-4C)alkyl; and

R¹¹ represents hydrogen or (1-4C)alkyl;

R¹² represents (1-4C)alkyl;

or a pharmaceutically acceptable salt thereof.

Included within the scope of the present invention are compounds of the formula X:

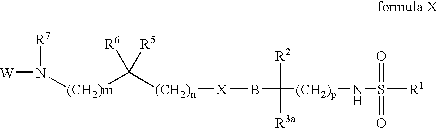

wherein

B represents

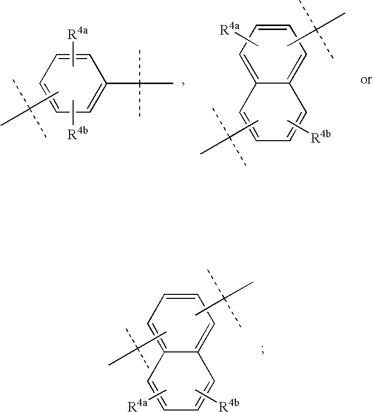

X represents O, NR, or S:

W represents R⁸SO₂—, R¹³C(═O)—, R¹³R¹⁵NC(═O)—, H₂NC(═O)—, R¹⁶, or R¹⁴OC(═O)—;

R represents hydrogen, (1-6C)alkyl, or -(1-4C)alkylaromatic;

R¹ represents (1-6C)alkyl, (2-6C)alkenyl, halo(1-4C)alkyl, or NR⁹R¹⁰;

R² and R^{3a} each independently represent hydrogen, (1-4C)alkyl, F, or —OR¹¹;

R^{3b} represents hydrogen, (1-4C)alkyl, or —OR¹²;

R^{4a} and R^{4b} each independently represent hydrogen, (1-4C) alkyl, (1-4C)alkoxy, I, Br, Cl, or F;

R⁵ and R⁶ each independently represent hydrogen, (1-4C)alkyl, F, or —OR¹¹;

R⁷ represents hydrogen, (1-4C)alkyl or -(1-4C)alkylaromatic;

R⁸ represents (1-6C)alkyl,-(1-4C)alkylphenyl, halo(1-4C)alkyl, unsubstituted or substituted aromatic group, unsubstituted or substituted heteroaromatic group, cycloalkyl, alkylcycloalkyl, or NR⁹R¹⁰;

n is zero or an integer 1, 2, 3, 4, or 5;

m is zero or an integer 1, 2, 3, 4, or 5;

p is an integer 1 or 2;

R⁹ and R¹⁰ each independently represent hydrogen or (1-4C)alkyl;

R¹¹ represents hydrogen or (1-4C)alkyl;

R¹² represents (1-4C)alkyl;

R¹³ represents phenyl or (1-6C)alkyl;

R¹⁴ represents (1-6C)alkyl;

R¹⁵ represents (1-4C)alkyl; and

R¹⁶ represents (1-4C)alkyl or -(1-4C)alkylphenyl;

or a pharmaceutically acceptable salt thereof, with the proviso that when W is R¹⁶, then B is other than

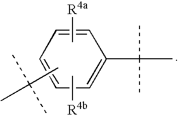

Further included within the scope of the present invention are compounds of the formula XX:

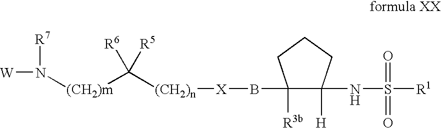

wherein

B represents

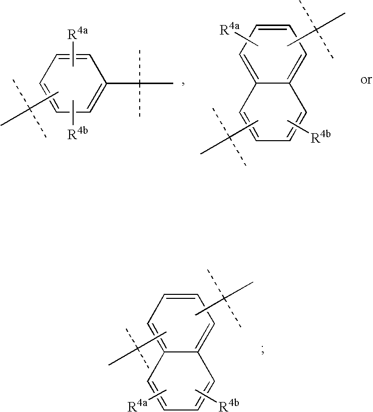

X represents O, NR, or S:

W represents R⁸SO₂—, R¹³C(═O)—, R¹³R¹⁵NC(═O)—, H₂NC(═)—, R¹⁶, or R¹⁴OC(═O)—;

R represents hydrogen, (1-6C)alkyl, or -(1-4C)alkylaromatic;

R¹ represents (1-6C)alkyl, (2-6C)alkenyl, halo(1-4C)alkyl, or NR⁹R¹⁰;

R² and R^{3a} each independently represent hydrogen, (1-4C)alkyl, F, or —OR¹¹;

R^{3b} represents hydrogen, (1-4C)alkyl, or —OR¹²;

R^{4a} and R^{4b} each independently represent hydrogen, (1-4C) alkyl, (1-4C)alkoxy, I, Br, Cl, or F;

R⁵ and R⁶ each independently represent hydrogen, (1-4C)alkyl, F, or —OR¹¹;

R⁷ represents hydrogen, (1-4C)alkyl or -(1-4C)alkylaromatic;

R⁸ represents (1-6C)alkyl,-(1-4C)alkylphenyl, halo(1-4C)alkyl, unsubstituted or substituted aromatic group, unsubstituted or substituted heteroaromatic group, cycloalkyl, alkylcycloalkyl, or NR⁹R¹⁰;

n is zero or an integer 1, 2, 3, 4, or 5;

m is zero or an integer 1, 2, 3, 4, or 5;

p is an integer 1 or 2;

R⁹ and R¹⁰ each independently represent hydrogen or (1-4C)alkyl;

R¹¹ represents hydrogen or (1-4C)alkyl;

R¹² represents (1-4C)alkyl;

R¹³ represents phenyl or (1-6C)alkyl;

R¹⁴ represents (1-6C)alkyl;

R¹⁵ represents (1-4C)alkyl; and

R¹⁶ represents (1-4C)alkyl or -(1-4C)alkylphenyl;

or a pharmaceutically acceptable salt thereof, with the proviso that when W is R¹⁶, then B is other than

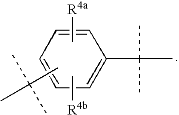

Further included within the scope of the present invention are compounds of the formula XXX:

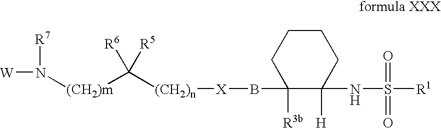

wherein

B represents

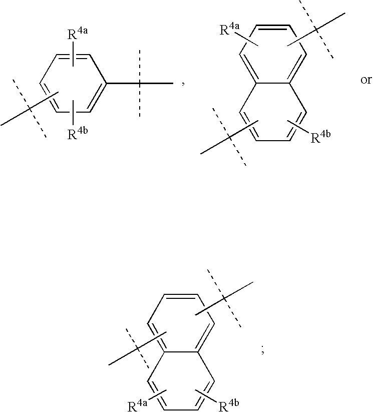

X represents O, NR, or S:

W represents R⁸SO₂—, R¹³C(═O)—, R¹³R¹⁵NC(═O)—, H₂NC(═O), R¹⁶, or R¹⁴OC(═O)—;

R represents hydrogen, (1-6C)alkyl, or —(1-4C)alkylaromatic;

R¹ represents (1-6C)alkyl, (2-6C)alkenyl, halo(1-4C)alkyl, or NR⁹R¹⁰;

R² and R^{3a} each independently represent hydrogen, (1-4C)alkyl, F, or —OR¹¹;

R^{3b} represents hydrogen, (1-4C)alkyl, or —OR¹²;

R^{4a} and R^{4b} each independently represent hydrogen, (1-4C) alkyl, (1-4C)alkoxy, I, Br, Cl, or F;

R⁵ and R⁶ each independently represent hydrogen, (1-4C)alkyl, F, or —OR¹¹;

R⁷ represents hydrogen, (1-4C)alkyl or -(1-4C)alkylaromatic;

R⁸ represents (1-6C)alkyl,-(1-4C)alkylphenyl, halo(1-4C)alkyl, unsubstituted or substituted aromatic group, unsubstituted or substituted heteroaromatic group, cycloalkyl, alkylcycloalkyl, or NR⁹R¹⁰;

n is zero or an integer 1, 2, 3, 4, or 5;

m is zero or an integer 1, 2, 3, 4, or 5;

p is an integer 1 or 2;

R⁹ and R¹⁰ each independently represent hydrogen or (1-4C)alkyl;

R¹¹ represents hydrogen or (1-4C)alkyl;

R¹² represents (1-4C)alkyl;

R¹³ represents phenyl or (1-6C)alkyl;

R¹⁴ represents (1-6C)alkyl;

R¹⁵ represents (1-4C)alkyl; and

R¹⁶ represents (1-4C)alkyl or -(1-4C)alkylphenyl;

or a pharmaceutically acceptable salt thereof, with the proviso that when W is R¹⁶, then B is other than

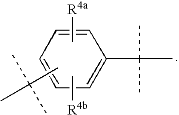

In this specification, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

A wide variety of conditions may be treated or prevented by compounds of formula I and their pharmaceutically acceptable salts through their action as potentiators of glutamate receptor function. Such conditions include those associated with glutamate hypofunction, such as psychiatric and neurological disorders, for example cognitive disorders and neuro-degenerative disorders such as Alzheimer's disease; age-related dementias; age-induced memory impairment; cognitive deficits due to autism, Down's syndrome and other central nervous system disorders with childhood onset, cognitive deficits post electroconvulsive therapy, movement disorders such as tardive dyskinesia, Hungtington's chorea, myoclonus, dystonia, spasticity, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, drug-induced psychosis, obesity, stroke, and sexual dysfunction. Compounds of formula I may also be useful for improving memory (both short term and long term) and learning ability. The present invention provides the use of compounds of formula I for the treatment of each of these conditions.

The present invention includes the pharmaceutically acceptable salts of the compounds defined by formula I. A compound of this invention can possess a sufficiently acidic group, a sufficiently basic group, or both functional groups, and accordingly react with-any of a number of organic and inorganic bases, and inorganic and organic acids, to form a pharmaceutically acceptable salt. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. Such salts include the pharmaceutically acceptable salts listed in Journal of Pharmaceutical Science, 66, 2-19 (1977), which are known to the skilled artisan. Acids commonly employed to form acid addition salts are inorganic acids such as hydrochloric acid, hydrobromic acid, hydriodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4dicarboxylate, hexyne-1,6-dioate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, 1-naphthalenesulfonate, 2-napththalenesulfonate, 1,5-naphthalenedisulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ⅇⅇ = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

As used herein, the term “aromatic group” means the same as aryl, and includes phenyl and a polycyclic aromatic carbocyclic ring such as 1- or 2-naphthyl, 1,2-dihydronaphthyl, 1,2,3,4-tetrahydronaphthyl, and the like.

The term “heteroaromatic group” includes an aromatic 5-6 membered ring containing from one to four heteroatoms selected from oxygen, sulfur and nitrogen, and a bicyclic group consisting of a 5-6 membered ring containing from one to four heteroatoms selected from oxygen, sulfur and nitrogen fused with a benzene ring or another 5-6 membered ring containing one to four atoms selected from oxygen, sulfur and nitrogen. Examples of heteroaromatic groups are thienyl, furyl, oxazolyl, isoxazolyl, oxadiazoyl, pyrazolyl, thiazolyl, thiadiazolyl, isothiazolyl, imidazolyl, triazolyl, tetrazolyl, pyridyl, pyridazinyl, pyrimidyl, benzofuryl, benzothienyl, benzimidazolyl, benzoxazolyl, benzothiazolyl, indolyl, and quinolyl.

The term “substituted” as used in the term “substituted aromatic or heteroaromatic group” herein signifies that one or more (for example one or two) substituents may be present, said substituents being selected from atoms and groups which, when present in the compound of formula I, do not prevent the compound of formula I from functioning as a potentiator of glutamate receptor function.

Examples of substituents which may be present in a substituted aromatic or heteroaromatic group include halogen; nitro; cyano; (1-10C) alkyl; (2-10C)alkenyl; (2-10C)alkynyl; (3-8C)cycloalkyl; halo(1-10C)alkyl; and (1-6C)alkoxy.

The term (1-10C)alkyl includes (1-8C)alkyl, (1-6C)alkyl and (1-4C)alkyl. Particular values are methyl, ethyl, propyl, isopropyl, butyl, isobutyl, t-butyl, pentyl, hexyl, heptyl, octyl, nonyl and decyl.

The term (2-10C)alkenyl includes (3-10C)alkenyl, (2-8C)alkenyl, (2-6C)alkenyl and (2-4C)alkenyl. Particular values are vinyl and prop-2-enyl.

The term (2-10C)alkynyl includes (3-10C)alkynyl, (2-8C)alkynyl, (2-6C)alkynyl and (3-4C)alkynyl. A particular value is prop-2-ynyl.

The term (3-8C)cycloalkyl, as such or in the term (3-8C)cycloalkyloxy, includes monocyclic and polycyclic groups. Particular values are cyclopropyl, cyclobutyl, cyclopentyl, cyclohexyl and bicyclo[2.2.2]octane. The term includes (3-6C)cycloalkyl: cyclopropyl, cyclobutyl, cyclopentyl and cyclohexyl.

The term (5-8C)cycloalkyl includes cyclopentyl, cyclohexyl, cycloheptyl and cyclooctyl.

The term hydroxy(3-8C)cycloalkyl includes hydroxy-cyclopentyl, such as 3-hydroxycyclopentyl.

The term oxo(3-8C)cycloalkyl includes oxocyclopentyl, such as 3-oxocyclopentyl.

The terms “halogen”, “Hal”, “halo”, or “halide” include fluorine, chlorine, bromine and iodine unless otherwise specified.

The term halo(1 -10C)alkyl includes halo(1-6C)alkyl, halo(1-4C)alkyl, fluoro(1-10C)alkyl, fluoro(1-6C)alkyl, fluoro(1-4C)alkyl, chloro(1-6C)alkyl and chloro(1-4C)alkyl, such as trifluoromethyl, 2,2,2-trifluoroethyl, and chloromethyl.

The term (1-10C)alkoxy includes (1-6C)alkoxy and (1-4C)alkoxy, such as methoxy, ethoxy, propoxy, isopropoxy and isobutoxy;

The term cyano(2-10C)alkenyl includes 2-cyanoethenyl.

The term (2-4C)alkylene includes ethylene, propylene and butylene. A preferred value is ethylene.

The term thienyl includes thien-2-yl and thien-3-yl.

The term furyl includes fur-2-yl and fur-3-yl.

The term oxazolyl includes oxazol-2-yl, oxazol-4-yl and oxazol-5-yl.

The term isoxazolyl includes isoxazol-3-yl, isoxazol-4-yl and isoxazol-5-yl.

The term oxadiazolyl includes [1,2,4]oxadiazol-3-yl and [1,2,4]oxadiazol-5-yl.

The term pyrazolyl includes pyrazol-3-yl, pyrazol-4-yl and pyrazol-5-yl.

The term thiazolyl includes thiazol-2-yl, thiazol-4-yl and thiazol-5-yl.

The term thiadiazolyl includes [1,2,4]thiadiazol-3-yl, and [1,2,4]thiadiazol-5-yl.

The term isothiazolyl includes isothiazol-3-yl, isothiazol-4-yl and isothiazol-5-yl.

The term imidazolyl includes imidazol-2-yl, imidazolyl-4-yl and imidazolyl-5-yl.

The term triazolyl includes [1,2,4]triazol-3-yl and [1,2,4]triazol-5-yl.

The term tetrazolyl includes tetrazol-5-yl.

The term pyridyl includes pyrid-2-yl, pyrid-3-yl and pyrid-4-yl.

The term pyridazinyl includes pyridazin-3-yl, pyridazin-4-yl, pyridazin-5-yl and pyridazin-6-yl.

The term pyrimidyl includes pyrimidin-2-yl, pyrimidin-4-yl, pyrimidin-5-yl and pyrimidin-6-yl.

The term benzofuryl includes benzofur-2-yl and benzofur-3-yl.

The term benzothienyl includes benzothien-2-yl and benzothien-3-yl.

The term benzimidazolyl includes benzimidazol-2-yl.

The term benzoxazolyl includes benzoxazol-2-yl.

The term benzothiazolyl includes benzothiazol-2-yl.

The term indolyl includes indol-2-yl and indol-3-yl.

The term quinolyl includes quinol-2-yl.

The term dihydrothiazolyl includes 4,5-dihydrothiazol-2-yl, and the term (1-4C)alkoxycarbonyldihydrothiazolyl includes 4-methoxycarbonyl-4,5-dihydrothiazol-2-yl.

The term -(1-4C)alkyl(3-8C)cycloalkyl includes the following:

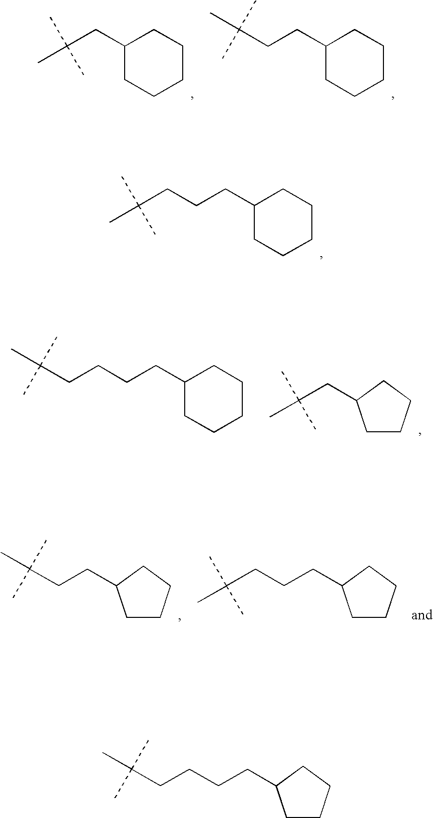

The term -(1-4C)alkylaromatic includes the following:

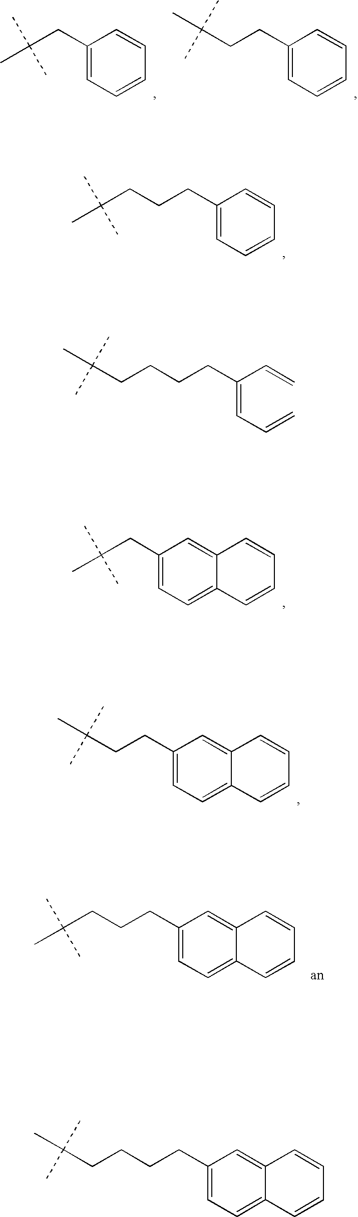

It is preferred that -(1-4C)alkylaromatic is -(1-4)alkylphenyl.

R¹ is preferably (1-6C)alkyl, with methyl, ethyl, propyl, 2-propyl, and butyl being most preferred, and 2-propyl being most especially preferred.

Examples of particular values for y are 0 and 1.

The compounds of the present invention can be prepared by one of ordinary skill in the art following art recognized techniques and procedures such as those that can be found, for example, in International Patent Application Publications: WO 98/33496 published Aug. 6, 1998; WO 99/43285 published Sep. 2, 1999; WO 00/06539; WO 00/06537, WO 00/06176, WO 00/06159, WO 00/06158, WO 00/06157, WO 00/06156, WO 00/06149, WO 00/06148, and WO 00/06083, all published Feb. 10, 2000; and WO 00/66546 published Nov. 9, 2000. More specifically, compounds of formula Ia and Ib can be prepared as set forth in Scheme I. The reagents and starting materials are lo readily available to one of ordinary skill in the art. All substituents, unless otherwise specified are as previously defined.

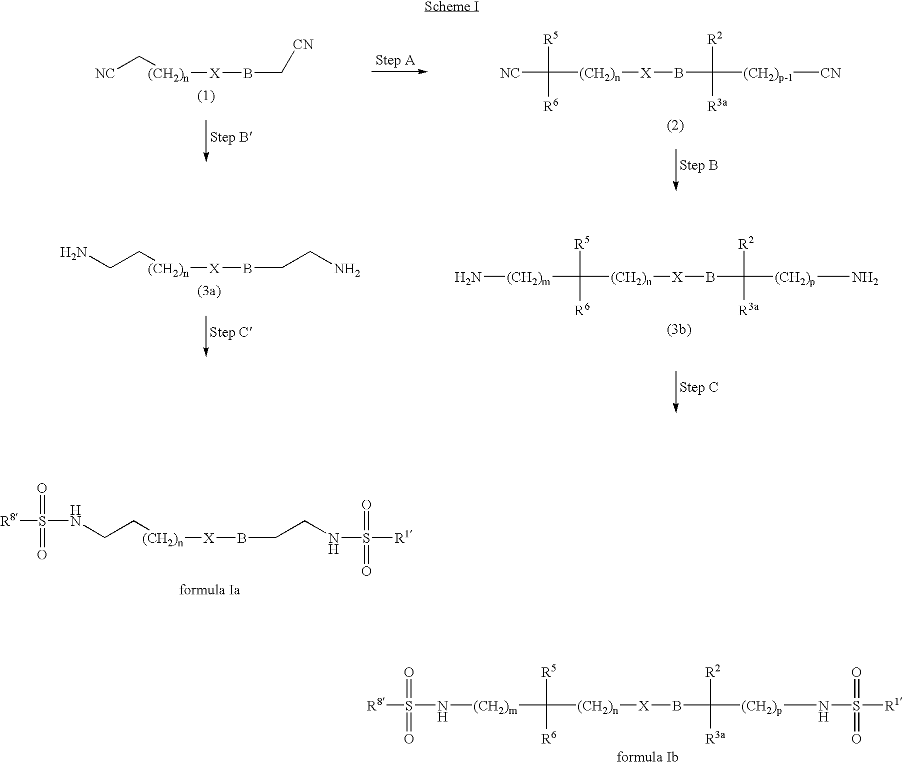

In Scheme I, step A the compound of structure (1) is alkylated under standard conditions to provide the compound of structure (2). For example, compound (1) is dissolved in a suitable organic solvent, such as THF, cooled to about −78° C. and treated with about 1.1 to 2.1 equivalents of a suitable base, such as hexamethylsilylazide. The mixture is stirred for about 30 minutes and then treated with about 1 to 2 equivalents of a suitable alkylating agent, such as iodomethane. The mixture is allowed to warm to room temperature and stirred for about 4 to 12 hours. The reaction is then quenched with water and extracted with a suitable organic solvent, such as ethyl acetate. The organic extracts are washed with water, brine, dried over anhydrous sodium sulfate, filtered, and concentrated under vacuum to provide the crude compound (2). This crude material can then be purified by chromatography on silica gel to with a suitable eluent, such as hexanes/ethyl acetate to provide the purified compound (2).

In Scheme I, step B compound (2) is reduced under standard conditions well known in the art to provide the compound (3b). For example, compound (2) is dissolved in a suitable organic solvent, such as THF and treated with about 2.1 equivalents of a suitable reducing agent, such as boron dimethylsulfide. The reaction is heated at reflux for about 4 to 14 hours, then cooled to room temperature, and quenched with a saturated solution of HCl in methanol. The quenched reaction mixture is then treated with a suitable organic solvent, such as diethyl ether, cooled to about 0° C., and the precipitated product (3b) collected by filtration as the dihydrochloride salt.

In Scheme I, step C, compound (3b) is sulfonylated under conditions well known in the art to provide the compound of formula Ib. For example, compound (3b) dissolved in a suitable organic solvent. Examples of suitable organic solvents include methylene chloride, tetrahydrofuran, and the like. The solution is treated with about 2.0 to about 5 equivalents of a suitable base, and then cooled to about −5° C. to about 0° C. Examples of suitable bases include triethylamine, pyridine, 1,8-diazabicyclo[5.4.0]undec-7-ene (DBU), and the like. To the stirring solution is added about 2.1 to about 2.3 equivalents of LgSO₂R^{1′} wherein R^{1′} and R^{8′} are equivalent The term “Lg” as used herein refers to a suitable leaving group. Examples of suitable leaving groups include, Cl, Br, and the like. Cl is the preferred leaving group. The reaction mixture is stirred at about 0° C. to about 25° C. for about 0.5 hours to about 16 hours. The compound of formula Ib is then isolated and purified by techniques well known in the art, such as extraction techniques and chromatography. For example, the mixture is washed with 10% sodium bisulfate, the layers separated and the aqueous extracted with several times with a suitable organic solvent, such as methylene chloride. The organic extracts are combined, dried over anhydrous sodium sulfate, filtered and concentrated under vacuum. The residue is then purified by flash chromatography on silica gel with a suitable eluent such as ethyl acetate/hexane to provide the purified compound of formula Ib.

In Scheme I, step B′, the compound (1) is reduced to the compound (3a) in a manner analogous to the procedure set forth in Scheme I, step B.

In Scheme I, step C′ the compound (3a) is sulfonylated to provide the compound of formula Ia in a manner analogous to the procedure set forth in Scheme I, step C.

Compounds of formula I′ can be prepared as set forth in Scheme II. The reagents and starting materials are readily available to one of ordinary skill in the art. All substituents, unless otherwise specified are as previously defined.

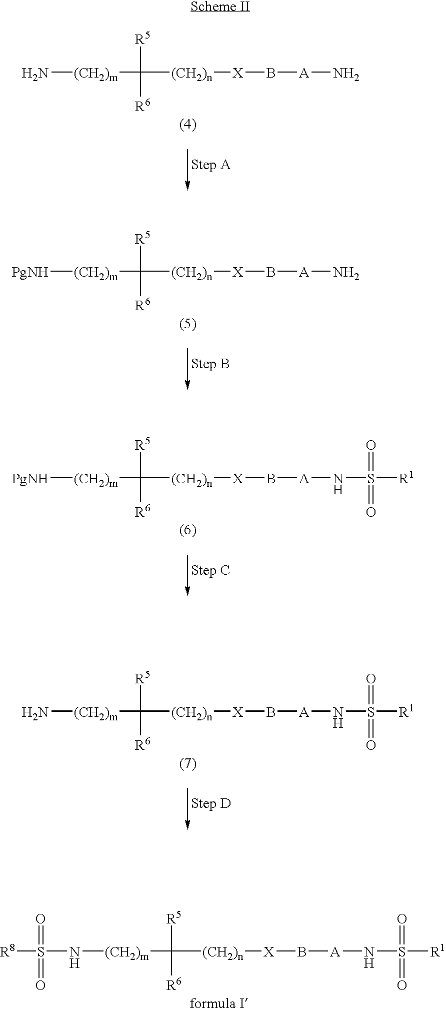

In Scheme II, step A, diamino compound (4) is protected with a suitable protecting group “Pg” under standard conditions to provide the mono-protected compound of structure (5). As used herein the term “Pg” refers to suitable protecting groups on the amine which are commonly employed to block or protect the amine while reacting other functional groups on the compound. Examples of suitable protecting groups used to protect the amino group and their preparation are disclosed by T. W. Greene, “Protective Groups in Organic Synthesis,” John Wiley & Sons, 1981, pages 218-287. Choice of the suitable protecting group used will depend upon the conditions that will be employed in subsequent reaction steps wherein protection is required, and is well within the knowledge of one of ordinary skill in the art. Preferred protecting groups are t-butoxycarbonyl also known as a BOC protecting group, and benzyloxycarbonyl, also known as CBz. For example, the diamino compound (4) is dissolved in a suitable organic solvent, such as methylene chloride and treated with about 1.2 equivalents of triethylamine. The solution is then cooled to about −5° C. and treated with one equivalent of as suitable protecting group, such as benzylchloroformate. The reaction mixture was warmed up to room temperature while stirring overnight. The reaction is then diluted with a suitable organic solvent, such as ethyl acetate, rinsed with water, brine, dried over anhydrous sodium sulfate, filtered, and concentrated under vacuum to provide the crude mono-protected compound (5) wherein Pg represents a CBz protecting group. The crude material can then be purified by techniques well known in the art, such as flash chromatography on silica gel with a suitable eluent, such as methanol/methylene chloride.

In Scheme II, step B, the protected compound (5) is sulfonylated with a compound of formula LgSO₂R¹ to provide sulfonamide (6) in a manner analogous to the procedure described in Scheme I, step C.

In Scheme II, step C, sulfonamide (6) is deprotected under conditions well known in the art as disclosed by T. W. Greene, “Protective Groups in Organic Synthesis,” John Wiley & Sons, 1981, pages 218-287 to provide the amino derivative of structure (7). The conditions employed for deprotection will depend upon the protecting group that needs to be removed and the substituents present on the compound itself which must remain unaffected by the deprotection reaction conditions, the conditions of which are well within the knowledge of one of ordinary skill in the art. For example, sulfonamide (6) wherein Pg represents a CBz protecting group is dissolved in a suitable organic solvent, such as ethanol and treated with a catalytic amount of 10% palladium on carbon. The reaction mixture is placed under an atmosphere of hydrogen for 2 to 12 hours and then filtered through Celite®. The filtrate is concentrated under vacuum and the crude amino derivative (7) is purified using standard techniques well known in the art, such as chromatography on silica gel with a suitable eluent, such as methanol/methylene chloride.

In Scheme II, step D, the amino compound (7) is sulfonylated with a compound of formula LgSO₂R⁸ to provide sulfonamide of formula I′ in a manner analogous to the procedure described in Scheme I, step C.

The compounds of structures (12) and (13) can be prepared following the procedures set forth in Scheme III below. The reagents and starting materials are readily available to one of ordinary skill in the art. All substituents, unless otherwise specified are as previously defined.

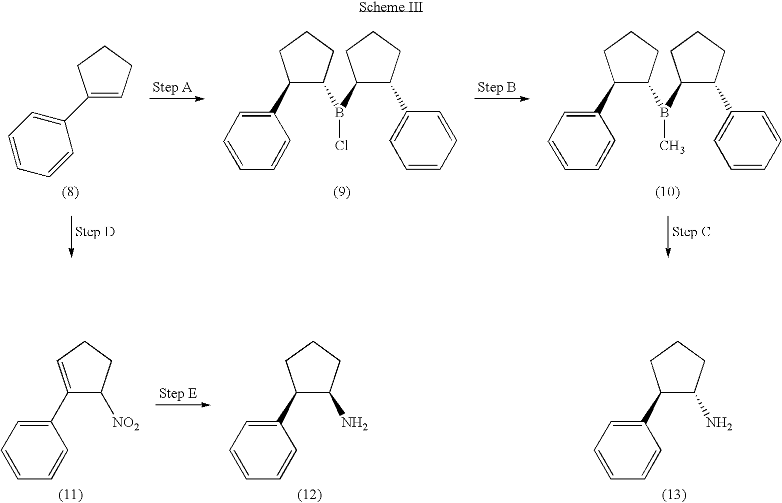

In Scheme III, step A, the cyclopentene of structure (8) is converted to the borane of structure (9) under standard conditions. For example, cyclopentene (8) is dissolved in a suitable organic solvent, such as dry methylene chloride under an atmosphere of nitrogen and cooled to about 0° C. The solution is treated with about 0.5 equivalents of monochloroborane-methyl sulfide. The reaction mixture is allowed to warm to room temperature and stirred for about 8 to 16 hours. The solvent is removed under vacuum under a nitrogen atmosphere to provide borane (9).

In Scheme III, step B, borane (9) is methylated to provide the methylborane of structure (10). For example borane (9) is dissolved in a suitable organic solvent, such as dry hexanes under an atmosphere of nitrogen. The solution is cooled to about 0° C. and treated with about 0.3 equivalents of trimethylaluminum in hexanes. The reaction mixture is allowed to warm to room temperature and stirred for about 1.5 hours. A precipitate results and the supernatant is transferred via cannula to a nitrogen flushed separatory funnel containing saturated aqueous ammonium chloride. The organic phase is then transferred via cannula to a flask containing anhydrous sodium sulfate. The organic solution is then transferred via cannula to a dry, nitrogen flushed flask and the solvent is removed under vacuum in the presence of a nitrogen atmosphere to provide the methylated borane (10).

In Scheme III, step C, the methylated borane (10) is hydrolyzed to the trans-cyclopentylamine of structure (13). For example, methylated borane (10) is dissolved in a suitable organic solvent, such as dry tetrahydrofuran and cautiously treated in small portions with a slight excess of hydroxylamine-O-sulfonic acid (referred to herein as “HAS”) dissolved in tetrahydrofuran. The reaction is exothermic. After addition is complete, the reaction mixture is stirred for about 24 hours and then filtered. The filtrate is concentrated under vacuum and the residue is treated with concentrated HCl:methanol:water:diethyl ether (30:15:20:60, by volume). The mixture is stirred at room temperature for about 30 minutes. The layers are separated, the organic phase is washed with water and the water wash is combined with the aqueous phase. The aqueous phase is cooled to about 0° C., diethyl ether is added and the aqueous is made basic with sodium hydroxide. The organic phase is separated and the aqueous phase is extracted with diethyl ether and ethyl acetate. The organic phase and organic extracts are combined, dried over anhydrous sodium sulfate, filtered and concentrated under vacuum to provide the cyclopentylamine (13).

In Scheme III, step D, the cyclopentene of structure (8) is nitrated under standard conditions to provide the compound of structure (11). For example, see the procedure disclosed by F. G. Bordwell, et al., J. Org. Chem., 1765-1769 (1963).

In Scheme III, step E, the nitrated compound of structure (11) is reduced under standard conditions to provide the amine of structure (12). For example, compound (11) is dissolved in a suitable organic solvent, such as ethanol, treated with a suitable hydrogenation catalyst, such as palladium on carbon, the solution is placed under hydrogen at about 413.69 kPa (60 psi). After about 8 to 16 hours, the reaction mixture is filtered and the filtrate is concentrated under vacuum to provide the compound (12).

The compound of structure (12) can be prepared by the alternative procedures set forth in Schemes IIIA and IIIB below. The reagents and starting materials are readily available to one of ordinary skill in the art. All substituents, unless otherwise specified are as previously defined.

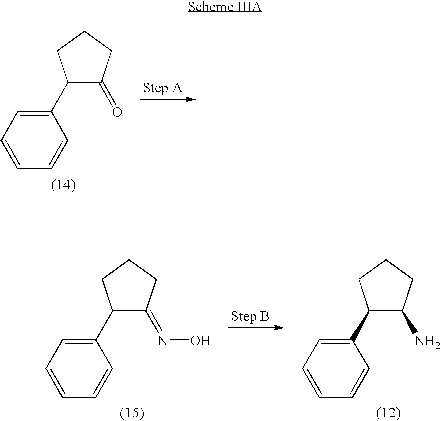

In Scheme IIIA, step A, the cyclopentanone of structure (14) is converted to the corresponding oxime of structure (15) under conditions well known in the art. For example, cyclopentanone (14) is dissolved in a suitable organic solvent, such as ethanol, treated with about 2 equivalents of aqueous sodium hydroxide and about 1.5 equivalents of hydroxylamine hydrochloride. The reaction mixture is stirred for about 8 to 16 hours at room temperature. It is then diluted with water and the precipitated oxime (15) is collected by filtration and dried under vacuum at about 35° C.

In Scheme IIIA, step B, oxime (15) is hydrogenated under standard conditions to provide the amine of structure (12). For example, oxime (15) is dissolved in a suitable organic solvent, such as ethanol, treated with a suitable catalyst, such as palladium on carbon, and placed under hydrogen at about 413.69 kPa (60 psi). The hydrogenation is carried out at about 40° C. for about 8 to 16 hours. The reaction mixture is then filtered and the filtrate concentrated under vacuum to provide the amine (12).

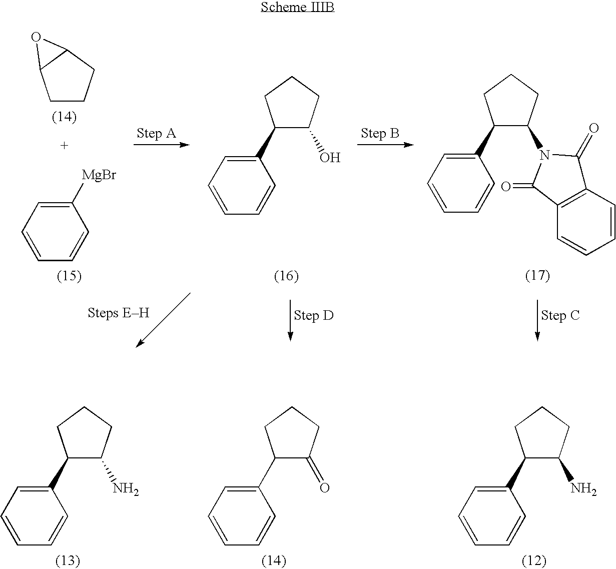

In Scheme IIIB, step A, the epoxide (14) is coupled with the Grignard reagent (15) to provide the alcohol (16). For example, Grignard (15) is dissolved in a suitable organic solvent, such as tetrahydrofuran and treated with a catalytic amount of copper iodide. To this solution is slowly added the epoxide (14) dissolved in tetrahydrofuran. The reaction is exothermic. The reaction is stirred until the temperature reaches room temperature and it is quenched with aqueous ammonium chloride. The quenched reaction is extracted with a suitable organic solvent, such as diethyl ether. The organic extracts are combined, washed with aqueous ammonium chloride, dried over anhydrous magnesium sulfate, filtered, and concentrated under vacuum to provide alcohol (16).

In Scheme IIIB, step B, alcohol (16) is converted to the compound of structure (17) under standard conditions well known in the art. For example, about one equivalent of triphenylphosphine is dissolved in a suitable organic solvent, such as tetrahydrofuran. The solution is cooled to about 0° C. and a solution of about one equivalent of diisopropyl azodicarboxylate in tetrahydrofuran is added dropwise to the solution with stirring. To this reaction mixture is added about one equivalent of phthalimide followed by addition of about one equivalent of alcohol (16) dissolved in tetrahydrofuran maintaining the temperature between about 5° C. and 0° C. The reaction is then stirred at about 0° C. for about 4 hours, warmed to room temperature, and stirred for 4 to 12 hours. The reaction is then quenched with water and extracted with a suitable organic solvent, such as chloroform. The organic extracts are combined, washed with water, dried over anhydrous magnesium sulfate, filtered, and concentrated under vacuum to provide compound (17).

In Scheme IIIB, step C, compound (17) is converted to compound (12) in an exchange reaction well known in the art. For example, compound (17) is dissolved in a suitable organic solvent, such as toluene, and an excess of anhydrous hydrazine is added dropwise over about 15 minutes with stirring. The reaction mixture is stirred for about one hour at room temperature and then heated at about 90-95° C. for about 6 hours. The reaction mixture is then cooled to room temperature, filtered, the precipitate rinsed with toluene, the filtrates combined, concentrated under vacuum to provide compound (12).

Alternatively, compound (17) is dissolved in 2-aminoethanol and heated at about 80-90° C. for about 1 to 2 hours. The reaction is then diluted with diethyl ether, washed with dilute sodium hydroxide, brine, dried over anhydrous sodium sulfate, filtered, and concentrated to provide compound (12).

In Scheme IIIB, step D, compound (16) oxidized to the ketone of structure (14) under standard conditions well known in the art. For example, compound (16) is added dropwise to a suspension of an excess of pyridinium chlorochromate in a suitable organic solvent, such as methylene chloride. The reaction is stirred for about 8 to 48 hours at room temperature. It is then diluted with a diethyl ether, filtered through a pad of silica gel and the filtrate concentrated under vacuum to provide crude compound (14). This material can be purified by standard techniques, such as flash chromatography on silica gel with a suitable eluent, such as ethyl acetate/hexane.

In Scheme IIIB, steps E through H, compound (16) is converted to the amine (13) using standard techniques and reactions well known in the art. For example, in step E, compound (16) is subjected to Mitsunobu conditions to provide the cis-benzoate derivative. More specifically, compound (16) is dissolved in a suitable organic solvent, such as THF and combined with about 1.05 equivalents of diethyl azodicarboxylate (referred to herein as “DEAD”), about 1.2 equivalents of benzoic acid and about 1.2 equivalents of triphenylphosphine at about 0° C. The reaction is stirred for about 2 hours, allowed to warm to room temperature and then concentrated under vacuum. The crude residue can be purified by chromatography on silica gel with a suitable eluent, such as hexanes/methylene chloride to provide the cis-benzoate derivative.

In Scheme IIIB, step F, the cis-benzoate is hydrolyzed under standard conditions to provide the cis-alcohol. For example, the cis-benzoate is combined with 5% NaOH/methanol and stirred at room temperature for about 3 hours. The reaction mixture is then concentrated under vacuum, the residue dissolved in a suitable organic solvent, such as diethyl ether, which is washed with water. The organic phase is then dried over potassium carbonate, filtered, and concentrated under vacuum. The residue can be purified by chromatography on silica gel with a suitable eluent, such as hexanes/methylene chloride to provide the cis-alcohol.

In Scheme IIIB, step G, the cis-alcohol is converted to the phthalimide derivative in a manner analogous to the procedure described above in Scheme IVB, step B.

In Scheme IIIB, step H, the phthalimide derivative is converted to the trans-amine (13) in a manner analogous to the procedure described above in Scheme IIIB, step C.

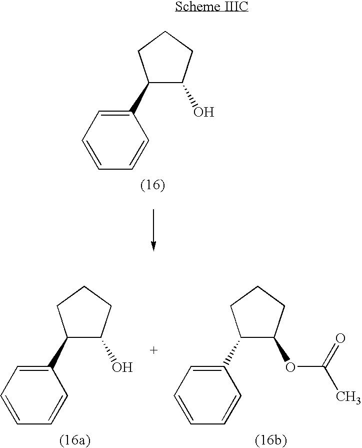

In Scheme IIIC, the compound (16) is subjected to an enzymatic resolution to provide the unreacted optically active alcohol (16a) and the optically active acetate (16b). For example, see the procedure described by Seemayer and Schneider, Recl. Trav. Chim. Pays-Bas, 110, 171-174 (1991), “Enzymatic Hydrolysis and Esterification. Routes to Optically Pure Cyclopentanols”. More specifically, the alcohol (16) is dissolved in a suitable organic solvent, such as tert-butyl methyl ether and combined with a suitable enzyme, such as Candida antartctica B lipase. With stirring, about 0.5 to about 0.6 equivalents of vinyl acetate is added and the reaction is stirred at room temperature for about 2 to 4 hours. The reaction mixture is then filtered and the filtrated is concentrated under vacuum to provide a mixture of the optically active alcohol (16a) and optically active acetate (16b). These compounds are then readily separated from each other using standard techniques well known in the art, such as flash chromatography on silica gel with a suitable eluent, such as ethyl acetate/hexane.

It is understood by one of ordinary skill in the art that the corresponding cyclohexyl derivatives of structures (12′) and (13′)

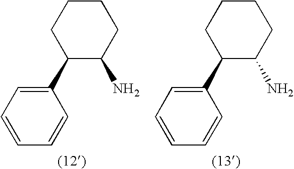

can be prepared in a manner analogous to the procedures set forth above.

Compounds of formula Ic can be prepared by the procedures set forth in Scheme IV. The reagents and starting materials are readily available to one of ordinary skill in the art. All substituents, unless otherwise specified are as previously defined.

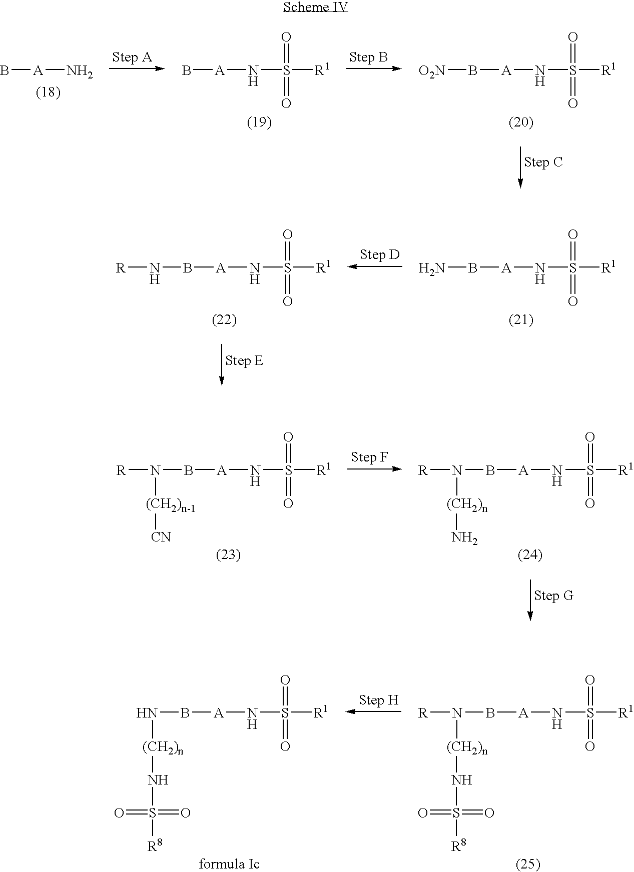

In Scheme IV, step A the compound of structure (18) is sulfonylated to provide the compound of structure (19) in a manner analogous to the procedure set forth in Scheme I, step C.

In Scheme IV, step B the compound (19) is nitrated under standard conditions well known in the art to provide the nitro derivative of structure (20). For example, compound (19) is dissolved in trifluoroacetic acid and treated with excess sodium nitrate. The reaction mixture is stirred for about 3 to 8 hours and then diluted with water. The quenched reaction mixture is extracted with a suitable organic solvent, such as methylene chloride, the organic extracts are combined, washed with saturated sodium carbonate, dried over anhydrous magnesium sulfate, filtered, and concentrated under vacuum to provide crude nitro derivative (20). This crude material can then be purified by chromatography on silica gel with a suitable eluent, such as hexanes/ethyl acetate.

In Scheme IV, step C, the nitro derivative (20) is reduced to the amine of structure (21) under standard conditions. For example, the nitro derivative (20) is dissolved in a suitable organic solvent, such as ethanol and treated with a suitable hydrogenation catalyst, such as 5% palladium on carbon. The mixture is placed under about 40 psi of hydrogen and agitated for 8 to 14 hours. The reaction mixture is then filtered through Celite® to remove the catalyst and the filtrate is concentrated under vacuum to provide the crude amine (21). The crude material can then be purified by chromatography on silica gel with a suitable eluent, such as hexanes/ethyl acetate.

In Scheme IV, step D, the amine (21) is alkylated under conditions well known in the art to provide the compound of structure (22). For example, amine (21) is dissolved in a suitable organic solvent, such as methanol, and treated with an equivalent of an aldehyde, such as benzaldehyde and treated with a catalytic amount of acetic acid. The reaction mixture is stirred for about 4 hours and then treated with about 2 equivalents of a suitable reducing agent, such as sodium borohydride. The reaction mixture is then stirred for about 8 to 14 hours at room temperature. The reaction is then diluted with water, extracted with a suitable organic solvent, the organic extracts are combined, dried over potassium carbonate, filtered, and concentrated under vacuum to provide the crude compound (22). This crude material can then be purified by chromatography on silica gel with a suitable eluent, such as hexanes/ethyl acetate.

In Scheme IV, step E, compound (22) is again alkylated under standard conditions well known in the art to provide the nitrile of structure (23). For example, compound (22) dissolved in a suitable organic solvent, such as methanol, is added to a solution of about 1.1 equivalents of sodium cyanide in water. The reaction mixture is cooled to about 0° C. and treated with hydrochloric acid and about 1.1 equivalents of formaldehyde is added. The reaction mixture is stirred for about 3 hours at 0° C., warmed to room temperature and stirred for about 14 hours. The reaction is then quenched with water and extracted with as suitable organic solvent, such as methylene chloride. The organic extracts are combined, washed with brine, dried over anhydrous magnesium sulfate, filtered, and concentrated under vacuum to provide the crude nitrile (23). The crude material can be purified by chromatography on silica gel with a suitable eluent, such as hexanes/ethyl acetate.

In Scheme IV, step F, the nitrile (23) is reduced to the amine of structure (24) in a manner analogous to the procedure described in Scheme I, step B.

In Scheme IV, step G, the amine (24) is sulfonylated to provide the compound of structure (25) in a manner analogous to the procedure described in Scheme I, step C.

In Scheme IV, step H, the compound (25) is dealkylated under standard conditions to provide the compound of formula Ic. For example, compound (25) is dissolved in a suitable organic solvent, such as THF, treated with a slight excess of ammonium formate and a catalytic amount of palladium oh carbon. The reaction mixture is stirred at room temperature for about 14 hours and then heated at reflux for about 8 hours. After cooling, the reaction mixture is filtered through Celite® which is washed is water. The filtrate is extracted with a suitable organic solvent, such as ethyl acetate. The organic extracts are combined, washed with brine, dried over anhydrous magnesium sulfate, filtered, and concentrated under vacuum to provide the crude compound of formula Ic. This crude material can then be purified by chromatography on silica gel with a suitable eluent, such as hexanes/ethyl acetate.

Compounds of formulas Id and Ie can be prepared by the procedures set forth in Scheme V. The reagents and starting materials are readily available to one of ordinary skill in the art. All substituents, unless otherwise specified are as previously defined.

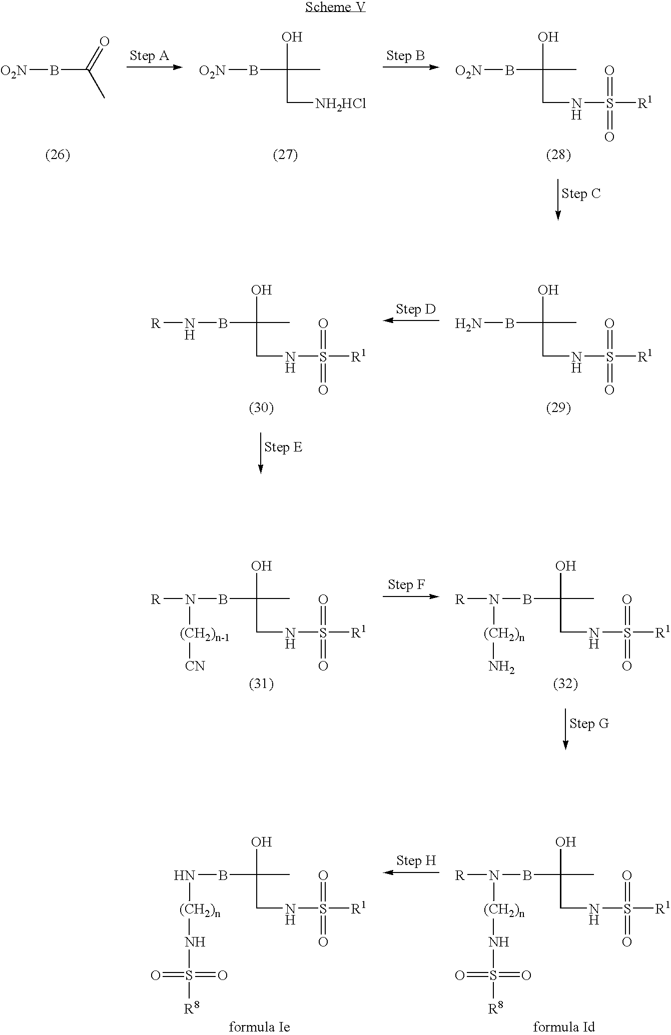

In Scheme V, step A, the compound of structure (26) is converted to the alcohol of structure (27) under standard conditions. For example, compound (26) is treated with excess trimethylsilylcyanate and zinc iodide under a nitrogen atmosphere at room temperature. The mixture is stirred for about 14 hours and treated with methylene chloride and saturated sodium carbonate. The layers are separated and the organic layer is washed with water, dried over anhydrous magnesium sulfate, filtered, and concentrated under vacuum. The residue is dissolved in a suitable organic solvent and treated with a suitable reducing agent, such as borane-dimethylsulfide. The reaction mixture is stirred at room temperature for about 14 hours and then treated with concentrated hydrochloric acid until a pH of about 2 is achieved. A suitable organic solvent, such as diethyl ether is then added and the resulting precipitate is collected by filtration, rinsed with diethyl ether and dried under vacuum to provide compound (27) as the hydrochloride salt.

In Scheme V, step B, the compound (27) is sulfonylated to provide the compound of structure (28) in a manner analogous to the procedure described in Scheme I, step C.

In Scheme V, step C, the compound (28) is reduced to provide the amine of structure (29) in a manner analogous to the procedure described in Scheme IV, step C.

In Scheme V, step D, the amine (29) is alkylated to provide the compound of structure (30) in a manner analogous to the procedure described in Scheme IV, step D.

In Scheme V, step E, the compound (30) is again alkylated to provide the nitrile of structure (31) in a manner analogous to the procedure described in Scheme IV, step E.

In Scheme V, step F, the nitrile (31) is reduced to the amine of structure (32) in a manner analogous to the procedure described in Scheme IV, step F.

In Scheme V, step G, the amine (32) is sulfonylated to provide the compound of formula Id in a manner analogous to the procedure described in Scheme IV, step G.

In Scheme V, step H, the compound of formula Id is dealkylated to provide the compound of formula Ie in a manner analogous to the procedure described in Scheme IV, step H.

Compounds of formula Ig can be prepared by the procedures set forth in Scheme VI. The reagents and starting materials are readily available to one of ordinary skill in the art. All substituents, unless otherwise specified are as previously defined.

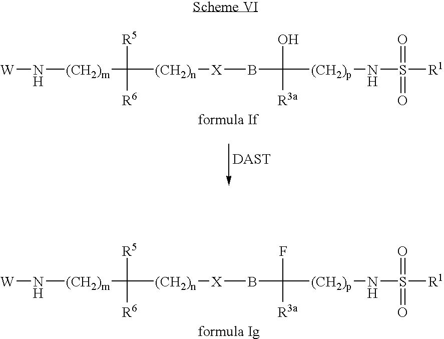

In Scheme VI the compound of formulas If is converted under conditions well known in the art to provide the corresponding fluorinated compound of formulas Ig. For example, For example, the compound of formula If is dissolved in a suitable organic solvent, such as methylene chloride and the solution is cooled to about −78° C. under an inert atmosphere, such as nitrogen. To this solution is added slowly, about one equivalent of diethylaminosulfur trifluoride (DAST) dissolved in a suitable organic solvent, such as methylene chloride with stirring. The reaction is then allowed to warm to room temperature and the compound of formula If or Ig is then isolated and purified using techniques and procedures well known in the art, such as extraction techniques and chromatography. For example, the reaction is diluted with water and methylene chloride. The layers are separated and the organic layer is washed with water, dried over anhydrous sodium sulfate, filtered and concentrated under vacuum to provide the crude compound of formula Ig. This crude material can then be purified by standard techniques, such as recrystallization from a suitable eluent, or flash chromatography or radial chromatography on silica gel, with a suitable eluent, such as hexane/ethyl acetate or methylene chloride to provide purified compound of formula Ig.

Compounds of formula I wherein W represents R¹³C(═O)—, R¹³R¹⁵NC(═O)—H₂NC(═O)—, R¹⁶, or R¹⁴OC(═O)— can further be prepared following the procedures set forth in Scheme VII. The reagents and starting materials are readily available to one of ordinary skill in the art. All substituents, unless otherwise specified are as previously defined.

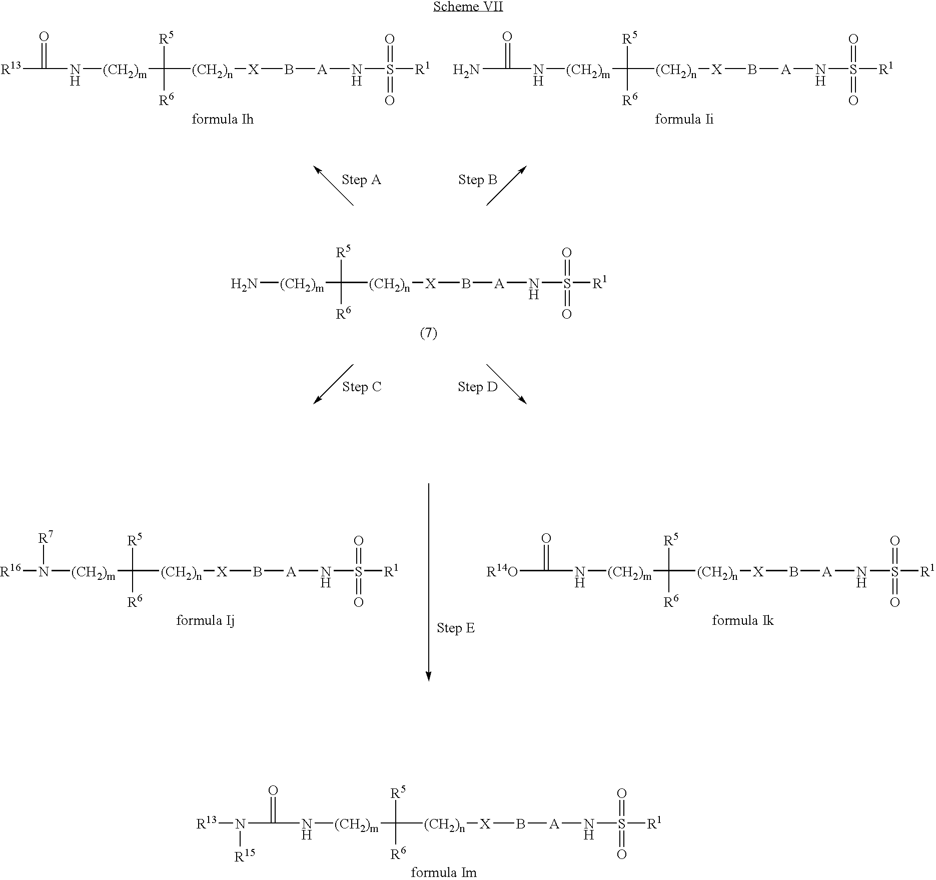

In Scheme I, step A the compound of structure (7) is acylated under standard conditions to provide the compound of formula Ih. For example, the compound (7) is dissolved in a suitable organic solvent, such as methylene chloride, the solution is cooled to about 0° C., and about 2 to 3 equivalents of a suitable base, such as triethylamine is added. The reaction is then treated with about 1.5 equivalents of a compound of formula R¹³C(═O)Lg wherein Lg is a leaving group, such as Cl or Br. Examples of compounds of formula R¹³C(═O)Lg are acetyl chloride, propionyl chloride, butyryl chloride, benzoyl chloride, and the like. The reaction mixture is then stirred for about 4 to 14 hours, quenched with water and the layers separated. The organic phase is rinsed with water, dried over anhydrous sodium sulfate, filtered, and concentrated under vacuum to provide the crude compound of formula Ih. This crude material can be purified by chromatography on silica gel with a suitable eluent, such as hexanes/ethyl acetate to provide the purified compound of formula Ih.

In Scheme VII, step B the compound (7) is converted to the compound of formula Ii under conditions well known in the art. For example, compound (7) is combined with about 2 equivalents of sodium cyanate in a suitable organic solvent, such as toluene and heated to about 50° C. The mixture is then treated with about 1.4 equivalents of trifluoroacetic acid and the mixture is heated to about 70° C. for about one hour. The reaction is then concentrated under vacuum and the residue is treated with aqueous sodium hydroxide and methylene chloride. The layers are separated and the organic phase is rinsed with brine, filtered through potassium carbonate, and the filtrate is concentrated under vacuum to provide the crude compound of formula Ii. This crude material can be purified by chromatography on silica gel with a suitable eluent, such as hexanes/ethyl acetate to provide the purified compound of formula Ii.

In Scheme VII, step C the compound (7) is converted to the secondary or tertiary amine of formula Ij under standard conditions well known in the art. The secondary amine can be prepared via reductive alkylation as described by Jerry March, “Advanced Organic Chemistry: Reactions, Mechanisms and Structure,” Fourth Edition, John Wiley & Sons, (1992), pages 898-900, reaction No. 6-15. For example, compound (7) is dissolved in a suitable organic solvent, such as methanol and treated with about one equivalent of an aldehyde or ketone, such as benzaldehyde and treated with about 0.05 equivalents of acetic acid (catalytic amount). The reaction mixture is stirred for about 2 to 8 hours and then treated with a suitable reducing agent, such as about 2 equivalents of sodium borohydride. The reaction mixture is then stirred for about 8 to 14 hours at room temperature and then diluted with water. The quenched reaction is extracted with a suitable organic solvent, such as methylene chloride, the organic extracts are combined, filtered through potassium carbonate, and concentrated under vacuum to provide the crude secondary amine of formula Ij. This crude material can be purified by chromatography on silica gel with a suitable eluent, such as ethyl acetate to provide the purified compound of formula Ij.

In addition, the tertiary amine can be prepared via dialkylation conditions as described by Jerry March, “Advanced Organic Chemistry: Reactions, Mechanisms, and Structure,” Fourth Edition, John Wiley & Sons, (1992), pages 411-413, 476 and 899-900. For example, compound (7) is dissolved in a suitable organic solvent, such as methanol and treated with an excess of formaldehyde. The reaction mixture is stirred at room temperature for about 1 to 3 hours. About 2 to 8 equivalents of a suitable reducing agent, such as sodium borohydride is then added and the reaction mixture is stirred for about 6 to 14 hours at room temperature. The reaction is then concentrated under vacuum and the residue is dissolved in water and a suitable organic solvent, such as methylene chloride. The layers are separated and the aqueous is extracted with methylene chloride. The organic layer and organic extracts are combined, washed with water and brine, dried over anhydrous magnesium sulfate, filtered and concentrated under vacuum to provide the crude tertiary amine of formula Ij. This crude material can be purified by chromatography on silica gel with a suitable eluent, such as methylene chloride:methanol to provide the purified compound of formula Ij.

In Scheme VII, step D compound (7) is converted to the carbamate of formula Ik under conditions well known in the art such as those described by Theodora W. Greene, “Protective Groups in Organic Synthesis,” John Wiley & Sons, (1981), Chapter 7. For example, compound (7) is dissolved in a suitable organic solvent such as methylene chloride, the solution is cooled to about 0° C. and about 2.5 equivalents of a suitable base, such as triethylamine is added. To this stirring solution is then added about 1.5 equivalents of a suitable chloroformate, such as methyl chloroformate, and the reaction is stirred for about 8 to 14 hours. The reaction is then quenched with water and the product is isolated and purified using standard techniques and procedures, such as extraction and chromatography. For example, the quenched reaction is extracted with a suitable organic solvent, such as methylene chloride, the combined organic extracts are dried over anhydrous sodium sulfate, filtered, and concentrated under vacuum to provide crude compound of formula Ik. This crude material can be purified by chromatography on silica gel with a suitable eluent, such as ethyl acetate to provide the purified compound of formula Ik.

In Scheme VII, step E compound (7) is converted to the substituted urea of formula Im under conditions well known in the art. For example, compound (7) is dissolved in a suitable organic solvent, such as methylene chloride, the solution is cooled to about 0° C. and treated with about 2.5 equivalents of a suitable base, such as triethylamine. To this stirring solution is then added about 1.5 equivalents of a carbamoyl chloride, such as N-methyl-N-phenylcarbamoyl chloride and the reaction is stirred for about 8 to 14 hours. The reaction is then quenched with water and the product is isolated and purified using standard techniques and procedures, such as extraction and chromatography. For example, the quenched reaction is extracted with a suitable organic solvent, such as methylene chloride, the combined organic extracts are dried over anhydrous sodium sulfate, filtered, and concentrated under vacuum to provide crude is compound of formula Im. This crude material can be purified by chromatography on silica gel with a suitable eluent, such as ethyl acetate to provide the purified compound of formula Im.

The following examples further illustrate the invention and represent typical syntheses of the compounds of formula I as described generally above. The reagents and starting materials are readily available to one of ordinary skill in the art. As used herein the term “Chromatotron®” (Harrison Research Inc., 840 Moana Court, Palo Alto Calif. 94306) is recognized by one of ordinary skill in the art as an instrument which is used to perform centrifugal thin-layer chromatography. As used herein, the following terms have the meanings indicated: “eq” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “aq” refers to aqueous; “iPrOAc” refers to isopropyl acetate; “MTBE” refers to tert-butyl methyl ether; “methyl DAST” refers to dimethylaminosulfur trifluoride, “DAST” refers to diethylaminosulfur trifluoride, “DBU” refers to 1,8-diazabicyclo[5.4.0]undec-7-ene; as used herein “Pd(dppf)₂Cl₂ catalyst” refers to ([1,1′-bis(diphenylphosphino)ferrocene]dichloropalladium(II) complex with CH₂Cl₂; as used herein the terms “Me”, “Et”, “Pr”, “iPr” and “Bu” refer to methyl, ethyl, propyl, isopropyl, and butyl respectively, and “RT” refers to room temperature.

### Preparation 1

### Chloro-bis-(2-phenyl-cyclopentyl)-borane

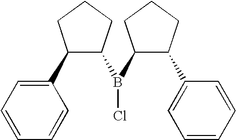

Scheme III, step A: Modification of H. C. Brown et. al., Tetrahedron Asymmetry, 7, 3527-3534 (1996). 1-Phenylcyclopentene (commercial 96%)(10.0 g, 69.4 mmol) was placed in an oven-dried flask under nitrogen and diluted with 60 mL of dry methylene chloride. The solution was cooled to 0° C. and monochloroborane-methyl sulfide complex (3.6 mL, 34.7 mmoL) was added dropwise via syringe. The solution was allowed to warm to room temperature and stirred overnight. The solvent is removed by aspirator vacuum under a nitrogen atmosphere to provide a crude colorless oil. This oil is used directly in the next step without further characterization.

### Preparation 2

### Methyl-bis-(2-phenyl-cyclopentyl)-borane

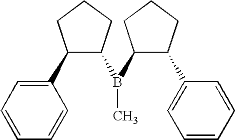

Scheme III, step B: Chloro-bis-(2-phenyl-cyclopentyl)borane from preparation 1 was diluted with 60 mL of dry hexanes under nitrogen. The solution was cooled to 0° C. and a 2M solution of trimethylaluminum in hexanes (5.8 mL) was added dropwise causing the reaction to turn orange. The reaction was allowed to warm to room temperature and stirred for 1.5 hours. During this time a red-brown mass precipitated out of solution, leaving a yellow supernatant. The hexane supernatant was transferred via cannula to a nitrogen flushed separatory funnel containing 50 mL of saturated aqueous ammonium chloride. The organic phase becomes colorless and was transferred via cannula to a dry flask containing sodium sulfate for drying. The solution was then transferred via cannula to a dry, nitrogen-flushed flask and the solvent removed under aspirator vacuum and nitrogen. The clear oil was used directly without further characterization.

### Preparation 3

### (+,−) Trans-2-phenyl-cyclopentylamine

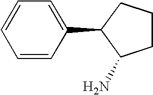

Scheme III, step C: Methyl-bis-(2-phenyl-cyclopentyl)-borane (theoretical 34.7 mmoL) from preparation 2 was diluted with 40 mL of dry tetrahydrofuran. 8.3 g (72.9 mmol) of hydroxylamine-O-sulphonic acid (HSA) was slurried in a separate dry flask in 60 mL of THF and small portions are transferred via cannula to control the exothermic reaction. The cloudy white solution was stirred at room temperature for 24 hours. The reaction mixture was filtered and the THF removed in vacuo. The residue was treated with 30 mL of concentrated HCl, 15 mL of methanol, 20 mL of water and 60 mL of diethyl ether and stirred at room temperature for 30 minutes. The aqueous phase was collected and the organic phase washed with water and combined with the aqueous phase. The aqueous phase was cooled to 0° C., layered with diethyl ether, and made strongly basic with sodium hydroxide pellets. The organic phase was separated and the aqueous phase extracted with diethyl ether (2×) and ethyl acetate (1×). The organic phases were combined and dried over sodium sulfate. The filtrate was concentrated to 5.96 (53%) of the title compound as a yellow oil.

Mass Spectrum (ES MS): M+1=162.

### Preparation 4

### (5-Nitro-cyclopent-1-enyl)-benzene

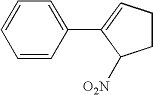

Scheme III, step D: (5-Nitro-cyclopent-1-enyl)-benzene was prepared according to the procedure of F. G. Bordwell et. al., J. Org. Chem., 1765-1769, 1963. The title compound was prepared by nitration of 1-phenylcyclopentene (3.0 g, 20.8 mmol) and purified by radial chromatography eluting with 85:15 hexanes:ethyl acetate to yield 0.63 g (12%) as a yellow oil.

### Preparation 5

### (+,−) Cis-2-phenyl-cyclopentylamine

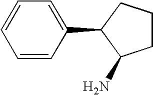

Scheme III, step E: (5-Nitro-cyclopent-1-enyl)-benzene (0.63 g, 3.3 mmol) from preparation 4 above, was hydrogenated in 25 mL of ethanol using 0.16 g of 5% Pd/C at room temperature overnight at 413.69 kPa (60 psi). The solution was filtered over celite and concentrated in vacuo to 230 mg (43%) of the title compound as a colorless oil.

Mass Spectrum (ES MS): M+1=162.

### Preparation 6

### (+,−) 2-Phenyl-cyclopentanone oxime

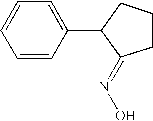

Scheme IIIA, step A: 2-Phenyl-cyclopentanone (prepared according to R. Sudha et. al. J. Org. Chem., 61, 1877-1879, 1996) (1.0 g, 6.2 mmol) was dissolved in 20 mL of absolute ethanol. To this solution was added sodium hydroxide (0.5 g, 12.5 mmol) dissolved in 10 mL water followed by hydroxylamine hydrochloride (0.65 g, 9.36 mmol) and stirred overnight at room temperature. The reaction was diluted with water and the precipitate collected by filtration. The white solid was vacuum oven-dried at 35° C. for 30 minutes to give 0.75 g (69%) of the title compound.

Analysis calculated for C₁₁H₁₃NO: % C, 75.40; % H, 7.48; % N, 7.99. Found: % C, 75.32; % H, 7.22; % N, 7.92. Mass Spectrum (ES MS): M+1=176.

### Preparation 7

### Alternative synthesis of (+,−) Cis-2-Phenyl-cyclopentylamine

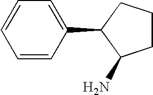

Scheme IIIA, step B: (+,−) 2-Phenyl-cyclopentanone oxime from preparation 6 above was dissolved in 35 mL of ethanol and hydrogenated using 90 mg of 5% Pd/C at 40° C. overnight at 413.69 kPa (60 psi). The solution was filtered and concentrated in vacuo to give 0.43 g (62%) of a colorless oil. Some dimeric material resulted by this procedure according to the mass spec. The cis:trans ratio was estimated to be 4:1. The amine was used directly without further purification. Mass Spectrum (ES MS): M+1=306, 162.

### Preparation 8

### Alternative synthesis of (+,−) Cis-2-Phenyl-cyclopentylamine

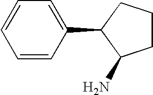

Scheme IIIB, step A: A one liter three necked round bottom flask equipped with a mechanical stirrer, addition funnel, thermometer is charged with 1M THF solution of phenylmagnesium bromide (300 mL, 300.0 mmol) and copper iodide (3.8 g, 20.0 mmol). To this reaction mixture was then added cyclopentene oxide (25.23 g, 300.0 mmol) dissolved in THF (50.0 mL) dropwise over a period of 60 minutes (reaction was quite exothermic, reaching THF reflux by the end of addition). The reaction mixture was then stirred to room temperature and quenched with 25% solution of ammonium chloride (200.0 mL). Added ether (80.0 mL) and separated upper organic layer. Washed organic layer with 25% ammonium chloride solution, dried with anhydrous magnesium sulfate, filtered and concentrated filtrate to provide (+,−) trans-2-phenyl-cyclopentanol as a brown oil (mass=47.7 g);

¹H nmr (CDCl₃) δ 1.6-1.8 (m, 4H), 2.0-2.2 (m, 2H), 2.8-2.88 (m, 1H), 4.13-4.16 (m, 1H), 7.2-7.4 (aromatic, 5H); ¹³C (CDCl₃) δ 22.46, 32.57, 34.64, 55.13, 81.11, 127.10, 128.11, 129.25, 144.05).

Scheme IIIB, step B: A 500 mL three necked round bottom flask equipped with a mechanical stirrer, thermometer, reflux condenser, addition funnel and a nitrogen blanket is charged with triphenylphosphine (16.19 g, 61.73 mmol) and THF (200 mL). To the solution at 0° C. was added dropwise, a solution of diisopropyl azodicarboxylate (12.15 mL, 61.73 mmol) dissolved in THF (30 mL) over a period of 10 minutes. A massive precipitate formed immediately after addition. To the slurry was then added solid phthalimide (9.08 g, 61.73 mmol), followed by a solution of 5-phenylcyclopentane-1-ol (10.0 g, 61.73 mmol) dissolved in THF (30 mL) over a period of 20 minutes maintaining temperature at 0° C. to 5° C. (reaction mixture went into solution by the end of alcoholic substrate addition). Reaction was then stirred at 0° C. for 4.0 hours and brought to room temperature overnight for convenience. Quenched reaction with water (200 mL) and extracted organics with chloroform (200 mL). Washed the organic with water (100 mL) and dried with anhydrous magnesium sulfate. Subsequent filtration and concentration under reduced pressure afforded an oil which solidified on equilibrating to room temperature. To the precipitate was then added hexane (250.0 mL) with vigorous stirring. Filtered off triphenylphosphine oxide precipitate and concentrated filtrate to an oil. Silica gel plug filtration of the oil with 1:1 ethyl acetate:hexanes and subsequent concentration of product fractions afforded an off white precipitate of (+,−) Cis-2-(2-phenyl-cyclopentyl)-isoindole-1,3-dione (mass=12.5 g, 69.6%);

¹H nmr (CDCl₃) δ 1.6-1.8 (m, 1H), 2.0-2.1 (m, 1H), 2.2-2.35 (m, 2H), 2.4-2.68 (m, 2H), 3.39-3.5 (m, 1H), 5.0-5.1 (m, 1H), 6.9-7.15 (aromatic, 5H), 7.52-7.64 (aromatic, 4H); ¹³C (CDCl₃) δ 25.4, 28.89, 30.56, 50.34, 54.60, 122.89, 126.44, 128.01, 128.41, 131.67, 139.68, 168.86).

Scheme IIIB, step C: A 1000 mL three necked flask equipped with a mechanical stirrer, thermometer, addition funnel and a reflux condenser is charged with (+,−) cis-2-(2-phenyl-cyclopentyl)isoindole-1,3-dione (27.34 g, 93.91 mmol) and toluene (400.0 mL). To this solution was added anhydrous hydrazine (29.48 mL, 939.09 mmol) dropwise over a period of 15 minutes. Stirred reaction at room temperature for 60 minutes then heated it at 90° C.-95° C. for 6.0 hours. Cooled. reaction to room temperature, filtered precipitates, washed cake with toluene (50.0 mL) and concentrated filtrate to provide the title compound as an oil (mass=15.13 g);

¹H nmr (CDCl₃) δ 0.6-0.8 (b, 1H), 1.5-1.6 (m, 1H), 1.63-1.69 (m, 1H), 1.9-2.0 (m, 2H, 2.0-2.1 (m, 2H), 3.05-3.1 (m, 1H), 3.4-3.7 (m, 1H), 7.19-7.35 (aromatic, 5H); ¹³C (CDCl₃) δ 23.05, 27.96, 34.98, 51.75, 56.68, 126.86, 128.96, 129.20, 142.00).

### EXAMPLE 1

### Preparation of [(methylethyl)sulfonyl]{2-[4-({[(methylethyl)sulfonyl]amino}methyl)phenoxy]propyl}amine

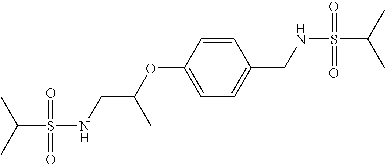

### Preparation of 4-(cyanoethoxy)benzenecarbonitrile

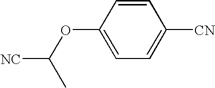

In a 250 mL round-bottomed flask fitted with a stir bar, at room temperature, and under a nitrogen atmosphere, a solution of 4-cyanophenol (2 g, 16.8 mmol) in acetone (75 mL) is treated with potassium carbonate (2.8 g, 20.2 mmol) and 2-bromopropionitrile (2.48 g, 18.5 mmol). The reaction mixture is heated at reflux (60° C.) overnight. The mixture is then allowed to cool to room temperature, acetone is removed in vacuum, 1N HCl (50 mL) is added and the organic is extracted with EtOAc (3×50 mL). The combined organic layer is washed with H₂O (2×50 mL), brine (50 mL), dried over anhydrous Na₂SO₄, filtered, and concentrated under reduced vacuum to yield crude product which is purified by recrystallization from CH₂Cl₂/Et₃N to yield the intermediate title compound (1.1 g, 38%). Electrospray MS 190 (M*+18).

### Preparation of 2-[4-(aminomethyl)phenoxy]propylamine dihydrochloride

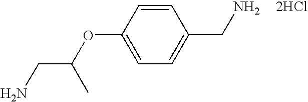

Scheme I, step B: Into a 100 mL single neck flask a solution of 4-(cyanoethoxy)benzenecarbonitrile (1 g, 5.8 mmol) in THF (20 mL) is treated with boron dimethylsulfide 10 M in THF (1.3 mL, 12.8 mmol) and the mixture is heated to reflux overnight. The reaction mixture is cooled down to room temperature and quenched with saturated solution of HCl in methanol (10 mL). Diethyl ether (20 mL) is added to the mixture and it is cooled down to 0° C. The product is precipitated out of the solution as dihydrochloride salt. The salt is filtered and dried in vacuum to provide the intermediate title compound (1.35 g, 88%) as a white solid crystal. Electron spray M.S. 181 (M*+1).

### Preparation of Final Title Compound

Scheme I, step C: Into a 25 mL single neck flask is placed 2-[4-(aminomethyl)phenoxy]propylamine dihydrochloride (0.5 g, 1.97 mmol) in methylene chloride (10 mL) and the solution is cooled down to 0° C. DBU, (1.75 mL, 11.8 mmol) is added to the mixture and after 30 minutes isopropylsulfonyl chloride (0.62 mL, 4.33 mmol) is added to the reaction mixture. The mixture is warmed up to RT while stirring for 12 hour. The reaction mixture is quenched with a 1 N HCl until pH is below 4-5. The product is extracted with CH₂Cl₂ (3×30 mL) and the combined organic layers are washed with H₂O (30 mL), dried over anhydrous Na₂SO₄, filtered, and concentrated under reduced vacuum. The resulting semi-solid is purified via flash chromatography (silica gel, gradient) and eluting with a solvent of Hexanes/EtOAc 40-45% to provide the final title compound (307 mg, 40%) as a white crystalline solid. Electron spray M.S. 391.1 (M*−1).

### EXAMPLE 2

### Preparation of [(methylethyl)sulfonyl]{2-[4-(2-{[(methylethylsulfonyl]amino}ethyl)phenoxy]propyl}amine

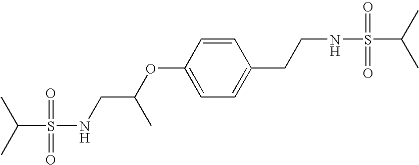

### Preparation of 2-[4-(cyanomethyl)phenoxy]propanenitrile

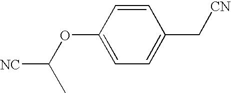

4-Hydroxybenzylcyanide (2 g, 15 mmol), Potassium carbonate (2.5 g, 18 mmol), and 2-bromopropionitrile (1.43 mL, 16.5 mmol), in acetone (65 mL) are combined in a manner analogous to the procedure described in example 1 to provide the intermediate title compound (2.1 g, 75%) as a white crystalline solid.

Electron spray M.S. 203.9 (M*+18).

### Preparation of 2-[4-(2-aminoethyl)phenoxy]propylamine dihydrochloride

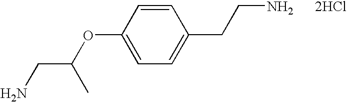

Scheme I, step B: 2-[4-(Cyanomethyl)phenoxy]propanenitrile (2 g, 10.7 mmol) in THF (50 mL) is treated with boron dimethylsulfide 2 M in THF (11.8 mL, 23.6 mmol) in a manner analogous to the procedure described in example 1 to provide the intermediate title compound (2.7 g, 95%) as a white crystalline solid.

Electron spray M.S. 195 (M+1).

### Preparation of Final Title Compound

Scheme I, step C: 2-[4-(2-Aminoethyl)phenoxy]propylamine dihydrochloride (1 g, 3.74 mmol), DBU (3.4 mL, 22.4 mmol), and isopropylsulfonyl chloride (0.92 mL, 8.2 mmol) in methylene chloride (20 mL) at 0° C. are combined in a manner analogous to the procedure described in example 1 to provide the final title compound (280 mg, 18%) as a white crystalline solid.

Electron spray M.S. 407 (M*+H).

Analysis for C₁₇H₃₀N₂O₅S₂:

| | | | |
|---|---|---|---|
| Theory: | C, 50.22 | H, 7.44 | N, 6.89 |
| Found: | C, 50.26 | H, 7.36 | N, 6.85 |

### EXAMPLE 3

### Preparation of [(methylethyl)sulfonyl]{2-[4-(3-{[(methylethyl)sulfonyl]amino}propyl)phenoxy]propyl}amine

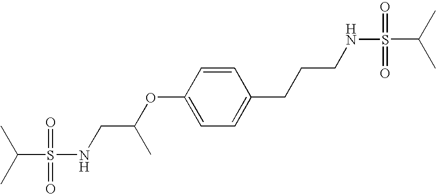

### Preparation of 2-[4-(2-cyanoethyl)phenoxy]propanenitrile

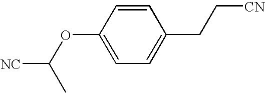

3-(4-Hydroxyphenyl)propionitrile (2 g, 13.6 mmol), potassium carbonate (2.25 g, 16.31 mmol), and 2-bromopropionitrile (1.3 mL, 14.95 mmol), in acetone (60 mL) are combined in a manner analogous to the procedure described in example 1 to provide the intermediate title compound (1.8 g, 67%) as a white crystalline solid. Electron spray M.S. 218 (M*+18).

### Preparation of 2-[4-(3-aminopropyl)phenoxy]propylamine dihydrochloride

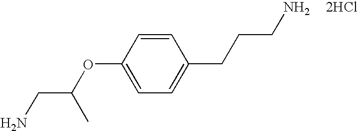

Scheme I, step B: 2-[4-(2-Cyanoethyl)phenoxy]propanenitrile (1 g, 5 mmol) in THF (20 mL) is treated with boron dimethylsulfide 2 M in THF (5.5 mL, 11 mmol) in a manner analogous to the procedure described in example 1 to provide the intermediate title compound (1.3 g, 93%) as a white crystalline solid.

Electron spray M.S. 209 (M+1).

### Preparation of the Final Compound

Scheme I, step C: 2-[4-(3-Aminopropyl)phenoxy]propylamine dihydrochloride (850 mg, 3 mmol), DBU (2.7 mL, 18 mmol), and isopropylsulfonyl chloride (0.74 mL, 6.6 mmol), in methylene chloride (15 mL) at 0° C. are combined in a manner analogous to the procedure described in example 1 to provide the final title compound (515 mg, 41 %) as a white crystalline solid.

Electron spray M.S. 421.1 (M*+H).

Analysis for C₁₈H₃₂N₂O₅S₂:

| | | | |
|---|---|---|---|
| Theory: | C, 51.40 | H, 7.67 | N, 6.66 |
| Found: | C, 51.43 | H, 7.62 | N, 6.63 |

### EXAMPLE 4

### Preparation of [(methylethyl)sulfonyl]{[4-(2-{[(methylethyl)sulfonyl]amino}ethoxy)phenyl]methyl}amine

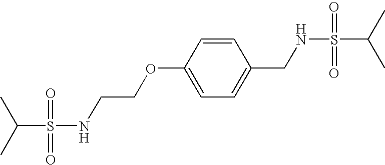

### Preparation of 4-(cyanomethoxy)benzenecarbonitrile

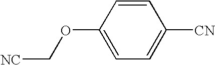

4-Cyanophenol (2 g, 16.8 mmol), potassium carbonate (2.8 g, 20 mmol), and bromoacetonitrile (1.3 mL, 18.45 mmol) in acetone (70 mL) are combined in a manner analogous to the procedure described in example 1 to provide the intermediate title compound (2.6 g, 98%) as a white crystalline solid. Electron spray M.S. 175.9 (M*+18).

### Preparation of 2-[4-(aminomethyl)phenoxy]ethylamine dihydrochloride

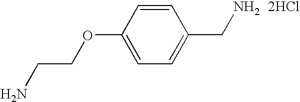

Scheme I, step B′: 4-(Cyanomethoxy)benzenecarbonitrile (1 g, 6.3 mmol) in THF (25 mL) is treated with boron dimethylsulfide 2 M in THF (7 mL, 13.9 mmol) in a manner analogous to the procedure described in example 1 to provide the intermediate title compound (1.4 g, 93%) as a white crystalline solid.

Electron spray M.S. 257 (M+18).

### Preparation of the Final Compound

Scheme I, step C′: 2-[4-(Aminomethyl)phenoxy]ethylamine dihydrochloride (500 mg, 2.1 mmol), DBU (1.9 mL, 12.5 mmol), and isopropylsulfonyl chloride (0.52 mL, 4.6 mmol), in methylene chloride (10 mL) at 0° C. are combined in a manner analogous to the procedure described in example 1 to provide the final title compound (126 mg, 16%) as a white crystalline solid. Electron spray M.S. 396 (M*+18).

Analysis for C₁₅H₂₆N₂O₅S₂:

| | | | |
|---|---|---|---|
| Theory: | C, 47.60 | H, 6.92 | N, 7.40 |
| Found: | C, 47.43 | H, 6.77 | N, 7.27 |

### EXAMPLE 5

### Preparation of [(methylethyl)sulfonyl]{2-[4-(2-{[(methylethyl)sulfonyl]amino}ethyl)phenoxy]ethyl}amine

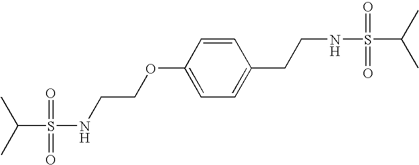

### Preparation of 2-[4-(cyanomethyl)phenoxy]ethanenitrile

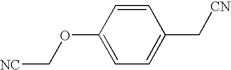

4-Hydroxybenzylcyanide (2 g, 15 mmol), potassium carbonate (2.5 g, 18 mmol), and bromoacetonitrile (1.15 mL, 16.5 mmol), in acetone (65 mL) are combined in a manner analogous to the procedure described in example 1 to provide the intermediate title compound (2.55 g, 98%) as a white crystalline solid.

Electron spray M.S. 172.1 (M*).

### Preparation of 2-[4-(2-aminoethoxy)phenyl]ethylamine dihydrochloride

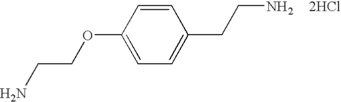

Scheme I, step B′: 2-[4-(Cyanomethyl)phenoxy]ethanenitrile (1 g, 5.8 mmol) in THF (25 mL) is treated with boron dimethylsulfide 2 M in THF (6.4 mL, 12.8 mmol) in a manner analogous to the procedure described in example 1 to provide the intermediate title compound (1.25 g, 85%) as a white crystalline solid.

Electron spray M.S. 181 (M+1).

### Preparation of the Final Compound

Scheme I, step C′: 2-[4-(2-Aminoethoxy)phenyl]ethylamine dihydrochloride (300 mg, 1.18 mmol), DBU (1.05 mL, 7.08 mmol), and isopropylsulfonyl chloride (0.29 mL, 2.6 mmol), in methylene chloride (6 mL) at 0° C. are combined in a manner analogous to the procedure described in example 1 to provide the final title compound (158 mg, 34%) as a white crystalline solid. Electron spray M.S. 393 (M*+H).

Analysis for C₁₆H₂₈N₂O₅S₂:

| | | | |
|---|---|---|---|
| Theory: | C, 48.96 | H, 7.19 | N, 7.14 |
| Found: | C, 49.25 | H, 7.27 | N, 7.18 |

### EXAMPLE 6

### Preparation of [(methylethyl)sulfonyl]{2-[4-(3-{[(methylethyl)sulfonyl]amino}propyl)phenoxy]ethyl}amine

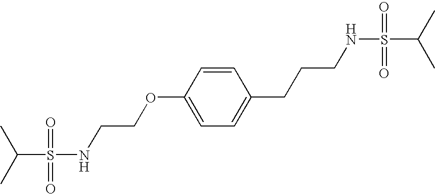

### Preparation of 3-[4-(cyanomethoxy)phenyl]propanenitrile

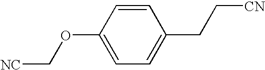

3-(4-Hydroxyphenyl)propionitrile (2 g, 13.6 mmol), potassium carbonate (2.25 g, 16.31 mmol), and bromoacetonitrile (1.04 mL, 14.95 mmol), in acetone (60 mL) are combined in a manner analogous to the procedure described in example 1 to provide the intermediate title compound (1.8 g, 72%) as a white crystalline solid. Electron spray M.S.186.1(M*+1).

### Preparation of 3-[4-(2-aminoethoxy)phenyl]propylamine dihydrochloride

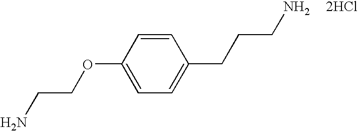

Scheme I, step B′: 3-[4-(Cyanomethoxy)phenyl]propanenitrile (1 g, 5.37 mmol) in THF (25 mL) is treated with boron dimethylsulfide 2 M in THF (6 mL, 11.8 mmol) in a manner analogous to the procedure described in example 1 to provide the intermediate title compound (1.33 g, 93%) as a white crystalline solid.

Electron spray M.S. 195 (M+1).

### Preparation of the Final Compound

Scheme I, step C′: 3-[4-(2-Aminoethoxy)phenyl]propylamine dihydrochloride (300 mg, 1.12 mmol), DBU (1 mL, 6.72 mmol), and isopropylsulfonyl chloride (0.28 mL, 2.47 mmol), in methylene chloride (6 mL) at 0° C. are combined in a manner analogous to the procedure described in example 1 to provide the final title compound (155 mg, 34%) as a white crystalline solid. Electron spray M.S. 407.1 (M*+1).

Analysis for C₁₇H₃₀N₂O₅S₂:

| | | | |
|---|---|---|---|
| Theory: | C, 50.22 | H, 7.44 | N, 6.89 |
| Found: | C, 49.94 | H, 7.30 | N, 6.84 |

### EXAMPLE 7

### Preparation of [(methylethyl)sulfonyl]{2-[4-(1-methyl-2-{[(methylethyl)sulfonyl]amino}ethyl)phenoxy]ethyl}amine

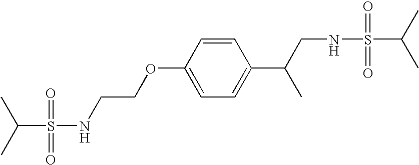

### Preparation of 2-[4-(cyanomethyl)phenoxy]ethanenitrile

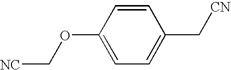

4-Hydroxybenzylcyanide (2 g, 15 mmol), potassium carbonate (2.5 g, 18 mmol), and 2-bromopropionitrile (1.15 mL, 16.5 mmol), in acetone (65 mL) are combined in a manner analogous to the procedure described in example 1 to provide the intermediate title compound (2.55 g, 98%) as a white crystalline solid.

Electron spray M.S. 172.1 (M*).

### Preparation of 2-[4-(cyanomethoxy)phenyl]propanenitrile

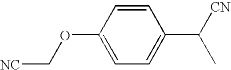

Scheme I, step A: In a 50 mL round-bottomed flask fitted with a stir bar, at room temperature, and under a nitrogen atmosphere, a solution of 2-[4-(cyanomethyl)phenoxy]ethanenitrile (1.4 g, 8.13 mmol) in THF (25 mL) is cooled down to −78 ° C. and is then treated with 1M solution of hexamethylsilylazide (8.9 mL, 8.9 mmol) in THF. The mixture is stirred at −78° C. for 30 minutes prior to the addition of iodomethane (0.51 mL, 8.13 mmol). The reaction mixture is allowed to warm up to RT overnight while stirring. Water (50 mL) is then added to the mixture and the organic is extracted with EtOAc (3×50 mL). The combined organic layers are washed with H₂O (2×50 mL), brine (50 mL), dried over anhydrous Na₂SO₄, filtered, and concentrated under reduced vacuum to yield crude product which is purified by flash chromatography (silica gel, isocratic) and eluting with a solvent of Hexanes/EtOAc 30% to provide the final title compound (690 mg, 46%) as a white crystalline solid. Electron pray M.S. 204.1 (M*+18).

### Preparation of 2-[4-(2-aminoethoxy)phenyl]propylamine dihydrochloride

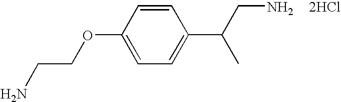

Scheme I, step B: 2-[4-(Cyanomethoxy)phenyl]propanenitrile (690 mg, 3.7 mmol) in THF (15 mL) is treated with boron dimethylsulfide 10 M in THF (0.815 mL, 8.15 mmol) in a manner analogous to the procedure described in example 1 to provide the intermediate title compound (800 mg, 81%) as a white crystalline solid. Electron spray M.S. 195 (M+1).

### Preparation of the Final Compound

Scheme I, step C: 2-[4-(2-Aminoethoxy)phenyl]propylamine dihydrochloride (500 mg, 1.87 mmol), DBU (1.67 mL, 11.2 mmol), and isopropylsulfonyl chloride (0.4 mL, 4.11 mmol), in methylene chloride (10 mL) at 0° C. are combined in a manner analogous to the procedure described in example 1 to provide the final title compound (415 mg, 54%) as a white crystalline solid. Electron spray M.S. 407.2 (M*+H).

Analysis for C₁₇H₃₀N₂O₅S₂:

| | | | |
|---|---|---|---|
| Theory: | C, 50.22 | H, 7.44 | N, 6.89 |
| Found: | C, 50.42 | H, 7.23 | N, 6.99 |

### EXAMPLE 8

### Preparation of [(methylethyl)sulfonyl]{2-[4-(2-{[(methylethyl)sulfonyl]amino}ethylthio)phenyl]ethyl}amine

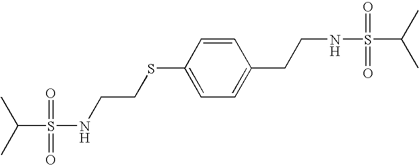

### Preparation of 2-[4-(ethoxythioxomethylthio)phenyl]ethanenitrile

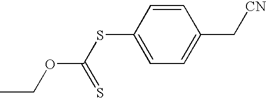

In a 100 mL round-bottomed flask fitted with a stir bar, at room temperature, and under a nitrogen atmosphere, a solution of 4-aminobenzylcyanide (5 g, 37.8 mmol) in HCl:H₂O (10:50 mL) is gradually treated with an aqueous solution of sodium nitrite (2.9 g, 41.6 mmol). The reaction mixture is stirred at 0° C. for 2 hours. This mixture is then added to a solution of potassium ethylxanthate (7.3 g, 46.4 mmol) in H₂O. The reaction mixture is then stirred overnight. Water is added to the mixture and the organic is extracted with EtOAc (3×50 mL). The combined organic layer is washed with H₂O (2×50 mL), brine (50 mL), dried over anhydrous Na₂SO₄, and filtered. The filtrate is concentrated under reduced vacuum to yield the crude product which is purified by flash chromatography (Silica gel, isocratic) and eluting with a solvent of Hexanes/EtOAc 15% to provide the intermediate title compound (2.6 g, 29%).

### Preparation of 2-(4-sulfanylphenyl)ethanenitrile

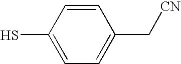

Into a 100 mL single neck flask a solution of 2-[4-(ethoxythioxomethylthio)phenyl]ethanenitrile (2.6 g, 11 mmol) in THF:MeOH:H₂O (25:15:10 mL) is treated with potassium hydroxide (1.85 g, 33 mmol) and the mixture is heated to 60° C. for 4 hours. The reaction mixture is then quenched with a 6N H₂SO₄. The product is extracted with EtOAc (3×20 mL) and the combined organic layer is washed with H₂O (30 mL), dried over anhydrous Na₂SO₄, filtered, and concentrated under reduced vacuum. The resulting crude product is purified via flash chromatography (Silica gel, gradient) and eluting with a solvent of Hexanes/EtOAc 15-25% to provide the intermediate title compound (576 mg, 36%) as a white solid crystal.

### Preparation of 2-[4-(cyanomethylthio)phenyl]ethanenitrile

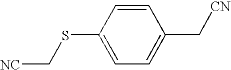

2-(4-Sulfanylphenyl)ethanenitrile (550 mg, 3.7 mmol), potassium carbonate (622 mg, 4.5 mmol), and bromoacetonitrile (280 μL, 4 mmol), in acetone (15 mL) are combined in a manner analogous to the procedure described in example 1 to provide the intermediate title compound (371 mg, 54%) as a white crystalline solid. Electron spray M.S. 187.0 (M*−1).

### Preparation of 2-[4-(2-aminoethylthio)phenyl]ethylamine dihydrochloride

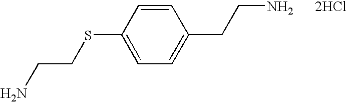

Scheme I, step B′: 2-[4-(Cyanomethylthio)phenyl]ethanenitrile (360 mg, 1.9 mmol) in THF (10 mL) is treated with boron dimethylsulfide 2 M in THF (2.1 mL, 4.2 mmol) in a manner analogous to the procedure described in example 1 to provide the intermediate title compound (500 mg, 98%) as a white crystalline solid. Electron spray M.S. 197.1 (M+1).

### Preparation of the Final Compound

Scheme I, step C′: 2-[4-(2-Aminoethylthio)phenyl]ethylamine dihydrochloride (500 mg, 1.85 mmol), DBU (1.7 mL, 11.1 mmol), and isopropylsulfonyl chloride (0.46 mL, 4.1 mmol) in methylene chloride (10 mL) at 0° C. are combined in a manner analogous to the procedure described in example 1 to provide the final title compound (392 mg, 52%) as a white crystalline solid.

Electron spray M.S. 409.2 (M*+H).

Analysis for C₁₆H₂₈N₂O₄S₃:

| | | | |
|---|---|---|---|
| Theory: | C, 47.03 | H, 6.91 | N, 6.86 |
| Found: | C, 47.29 | H, 6.97 | N, 6.88 |

### EXAMPLE 9

### Preparation of [(methylethyl)sulfonyl](2-{4-[methyl(2-{[(methylethyl)sulfonyl]amino}ethyl)amino]phenyl}ethyl)amine

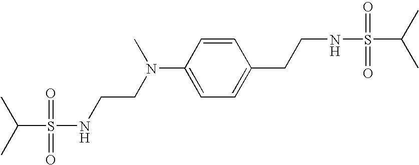

### Preparation of 2-{4-[(cyanomethyl)amino]phenyl}ethanenitrile

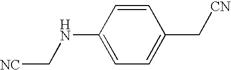

In a 100 mL round-bottomed flask fitted with a stir bar, at room temperature, and under a nitrogen atmosphere, a solution of 4-aminobenzylcyanide (2 g, 15.1 mmol) in methanol (30 mL) is treated with a solution of sodium cyanide (0.74 g, 15.1 mmol) in water (20 mL). The reaction mixture is cooled to 0° C. The mixture is treated first with 5N HCl (3 mL) and then with formaldehyde (1.25 mL, 15.1 mmol). The reaction mixture is stirred at 0° C. for 3 hours and at room temperature overnight. The reaction is poured into H₂O and the organic is extracted with CH₂CL₂ (3×25 mL). The combined organic layer is washed with H₂O (2×50 mL), brine (50 mL), dried over anhydrous Na₂SO₄, filtered, and concentrated under reduced vacuum to provide the intermediate title compound (2.5 g, 97%). Electrospray MS 189.0 (M*+18).

### Preparation of 2-{4-[(cyanomethyl)methylamino]phenyl}ethanenitrile

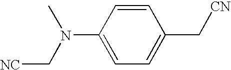

In a 25 mL round-bottomed flask fitted with a stir bar, at room temperature, and under a nitrogen atmosphere, a solution of 2-{4-[(cyanomethyl)amino]phenyl}ethanenitrile (1 g, 5.8 mmol) in formaldehyde (4 mL) and formic acid (4 mL) is prepared. The reaction mixture is refluxed for 2 hours. The mixture was cooled to room temperature. The mixture is basified and poured into H₂O and the organic was extracted with EtOAc (3×20 mL). The combined organic layer is washed with H₂O (2×20 mL), brine (20 mL), dried over anhydrous Na₂SO₄, filtered, and concentrated under reduced vacuum, The resulting semi-solid is purified via flash chromatography (Silica gel, gradient) and eluting with a solvent of Hexanes/EtOAc 25-35% to provide the intermediate title compound (610 mg, 57%). Electrospray MS 186.0(M*+1).

### Preparation of (2-aminoethyl)[4-(2-aminoethyl)phenyl]methylamine

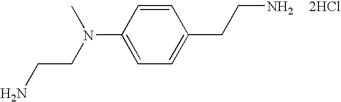

Scheme I, step B′: 2-{4-[(Cyanomethyl)methylamino]phenyl}ethanenitrile (600 mg, 3.24 mmol) in THF (15 mL) is treated with boron dimethylsulfide 2 M in THF (3.6 mL, 7.13 mmol) in a manner analogous to the procedure described in example 1 to provide the intermediate title compound as a white crystalline solid.

### Preparation of the Final Compound

Scheme I, step C′: (2-Aminoethyl)[4-(2-aminoethyl)phenyl]methylamine (1 g, 8.3 mmol), DBU (3.4 mL, 22.5 mmol), and isopropylsulfonyl chloride (0.932 mL, 4.1 mmol), in methylene chloride (15 mL) at 0° C. are combined in a manner analogous to the procedure described in example 1 to provide the final title compound (72 mg, 5%) as a white crystalline solid. Electron spray M.S. 409.2 is (M*+H).

Analysis for C₁₇H₃₁N₃O₄S₂:

| | | | |
|---|---|---|---|
| Theory: | C, 50.35 | H, 7.70 | N, 10.36 |
| Found: | C, 50.42 | H, 7.59 | N, 10.18 |

### EXAMPLE 10

### Preparation of [(methylethyl)sulfonyl](2-{4-[(2-{[(methylethyl)sulfonyl]amino}ethyl)benzylamino]phenyl}ethyl)amine

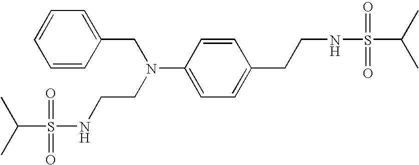

### Preparation of 2-{4-[benzylamino]phenyl}ethanenitrile

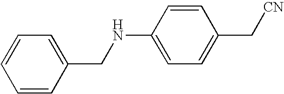

In a 250 mL round-bottomed flask fitted with a stir bar, at room temperature, and under a nitrogen atmosphere, a solution of 4-aminobenzylcyanide (2.3 mL, 22.7 mmol) is treated with benzaldehyde (0.74 g, 22.7 mmol) and a few drops of acetic acid. The reaction mixture is stirred at room temperature for 5 hours. The mixture is treated with sodium borohydride (1.6 g, 44 mmol) and the reaction mixture is stirred at room temperature overnight. The reaction is poured into H₂O and the organic is extracted with CH₂CL₂ (3×50 mL). The combined organic extracts are washed with H₂O (2×50 mL), brine (50 mL), dried over anhydrous Na₂SO₄, filtered, and concentrated under reduced vacuum to yield crude product (5 g, 100%). Electrospray MS 223 (M*+1).

### Preparation of 2-{4-[(cyanomethyl)benzylamino]phenyl}ethanenitrile

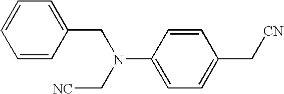

2-{4-[Benzylamino]phenyl}ethanenitrile (2 g, 9 mmol), formaldehyde (0.75 mL, 9 mmol), sodium cyanide (440 mg, 9 mmol), and 5N HCl (1.8 mL) in MeOH:H₂O (15:15 mL) at 0° C. are combined in a manner analogous to the procedure described in example 9 to provide the intermediate title compound(2.25 g, 96%). Electron spray M.S. 262.1 (M*+H).

### Preparation of (2-aminoethyl)[4-(2-aminoethyl)phenyl]benzylamine dihydrochloride

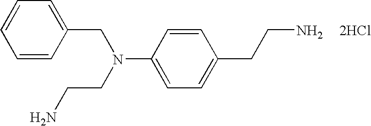

Scheme I, step B′: 2-{4-[(Cyanomethyl)benzylamino]phenyl}ethanenitrile (1 g, 3.8 mmol) in THF (20 mL) is treated with boron dimethylsulfide 2 M in THF (4.2 mL, 8.4 mmol) in a manner analogous to the procedure described in example 1 to provide the intermediate title compound (1.3 g, 100%) as a white crystalline solid. Electron spray M.S. 270.1 (M*+H).

### Preparation of the Final Compound

Scheme I, step C′: (2-Aminoethyl)[4-(2-aminoethyl)phenyl]benzylamine dihydrochloride (1.3 g, 3.8 mmol), DBU (3.4 mL, 22.8 mmol), and isopropylsulfonyl chloride (0.95 mL, 8.4 mmol), in methylene chloride (20 mL) at 0° C. are combined in a manner analogous to the procedure described in example 1 to provide the final title compound (588 mg, 33%) as a white crystalline solid. Electron spray M.S. 482.3 (M*+H).

Analysis for C₂₃H₃₅N₃O₄S₂:

| | | | |
|---|---|---|---|
| Theory: | C, 57.35 | H, 7.32 | N, 8.72 |
| Found: | C, 57.35 | H, 7.40 | N, 8.71 |

### EXAMPLE 11

### Preparation of [(methylethyl)sulfonyl](2-{4-[(2-{[(methylethyl)sulfonyl]amino}ethyl)amino]phenyl}ethyl)amine

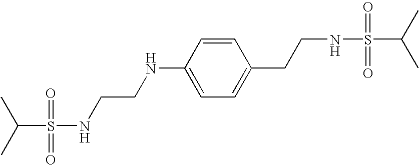

Scheme I, step C′: Into a 25 mL single neck flask is placed [(methylethyl)sulfonyl](2-{4-[(2-{[(methylethyl)sulfonyl]amino}ethyl)benzylamino]phenyl}ethyl)amine (0.465 g, 0.97 mmol) in THF (5 mL) and the solution is treated with aqueous solution of 5M ammonium formate (2 mL, 4.8 mmol) and catalytic amount of the palladium on carbon. The mixture is stirred at room temperature under atmospheric hydrogen for 12 hours. The reaction mixture is filtered over a layer of Celite®, water is added to the filtrate and the organic is extracted with EtOAc (3×20 mL). The combined organic extracts are washed with H₂O (30 mL), dried over anhydrous Na₂SO₄, filtered, and concentrated under reduced vacuum. The resulting semi-solid is purified via flash chromatography (Silica gel, gradient) and eluting with a solvent of Hexanes/EtOAc 35-45% to provide the final title compound (257 mg, 68%) as a white crystalline solid. Electron spray M.S. 392.1 (M*+1).

Analysis for C₁₆H₂₉N₃O₄S₂:

| | | | |
|---|---|---|---|
| Theory: | C, 49.08 | H, 7.47 | N, 10.73 |
| Found: | C, 48.83 | H, 7.40 | N, 10.72 |

### EXAMPLE 12

### Preparation of cis-[(methylethyl)sulfonyl](2-{[4-(2-{[(methylethyl)sulfonyl]amino}cyclopentyl)phenyl]amino}ethyl)amine

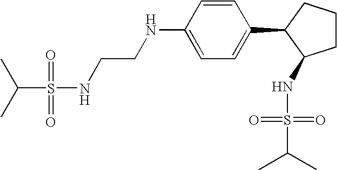

### Preparation of trans-2-phenylcyclopentan-1-ol

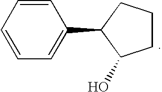

Scheme IIIB, step A: In a 3-neck round bottomed flask fitted with a condenser, a thermometer, a mechanical stirrer, a solution of 1,2-epoxy cyclopentane (25.0 g, 297.2 mmol) in tetrahydrofuran (60 mL) is added dropwise to a mixture of phenylmagnesium bromide (99.1 mL) and copper (II) iodide (4.0 g, 20.8 mmol). After an hour of addition, the temperature inside the vessel exceeds the reflux temperature of 60° C., and the reaction mixture ceases foaming. The reaction mixture is cooled down to room temperature overnight and is then treated dropwise with a 25% solution of ammonium chloride (45 mL) until ‘blue’ copper chloride precipitates out. The organic layer is washed with H₂O, filtered it through magnesium sulfate (MgSO₄), and concentrated under vacuum to yield 26.61 g of the crude product. This crude material is further purified by Prep HPLC 2000 (Hexanes:EtOAc, 3:1, isocratic) to provide the intermediate title compound (22.4 g, 46.5 %) as an orange oil.

### Preparation of cis-2-(2-phenylcyclopentyl)isoindoline-1,3-dione

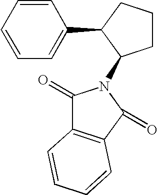

Scheme IIIB, step B: In a 2-Liter 3-neck round bottomed flask fitted with a condenser, a thermometer, and a mechanical stirrer a solution of diisopropyl azodicarboxylate (95 mL, 481.4 mmol) in THF (100 mL) is added to a solution of triphenylphosphine (126.3 g, 481.4 mmol) in THF (1400 mL). This mixture is treated with a phthalimide (70.8 g, 481.4 mmol) and a solution of trans-2-phenylcyclopentan-1-ol (78.1 g, 481.4 mmol) in THF (100 mL). The reaction temperature is kept between 0° C. and 5° C. during the addition and the mixture is warmed up to room temperature gradually. The reaction mixture is then quenched with H₂O (460 mL), and extracted with CH₂Cl₂ (3×300 mL). The combined organic extracts are washed with H₂O, filtered through magnesium sulfate, and concentrated under vacuum, yielding 330 g of brown oil. This crude material is washed with hexanes to yield 60 g of yellow oil. This material is further purified by flash chromatography (CH₂Cl₂:Hexanes, 3:1) to provide the intermediate title compound (30.8 g, 22%) as a white solid.

### Preparation of cis-2-phenylcyclopentylamine

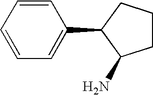

Scheme IIIB, step C: In a 2-Liter 3-neck round bottomed flask fitted with a mechanical stirrer, condenser, thermometer, a solution of cis-2-(2-phenylcyclopentyl)isoindoline-1,3-dione (30.8 g, 105.72 mmol) in toluene (423 mL, Aldrich) is treated with hydrazine (33.2 mL, 105.72 mmol). The mixture is heated at reflux (90° C.) for 6 hours and then cooled to room temperature. The resulting precipitate is filtered and rinsed with an additional 200 mL toluene. The combined filtrate is concentrated under vacuum yielding 18.6 g of yellow oil. The crude material is purified by flash chromatography (CH₂Cl₂:methanol, 9:1) to provide the intermediate title compound (11.52 g, 68%) as a yellow oil.

### Preparation of cis-[(methylethylsulfonyl](2-phenylcyclopentyl)amine

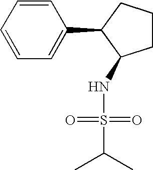

Scheme IV, step A: In a 2-Liter, 3-neck, round bottomed flask fitted with a thermometer, a solution of cis-2-phenylcyclopentylamine (11.52 g, 71.42 mmol,) in CH₂Cl₂ (476 mL) is treated with DBU (10.7 mL, 71.42 mmol) via additional funnel. The reaction is cooled down to 0° C. and isopropylsulfonyl chloride (8.0 mL, 71.42 mmol) is added. The mixture is gradually warmed to room temperature while stirring over night. The reaction mixture is then quenched with H₂O (476 mL), and the mixture is extracted with CH₂Cl₂ (2×300 mL). The combined organic extracts are washed with brine (500 mL), filtered through magnesium sulfate, and concentrated under vacuum to yield 11.0 g of a yellow oil. This crude material is further purified by flash chromatography (Hexanes:EtOAc, 3:1) to provide the intermediate title compound (9.30 g, 49%) as white solid.

### Preparation of cis-[(methylethyl)sulfonyl][2-(4-nitrophenyl)cyclopentyl]amine

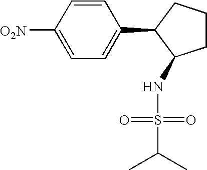

Scheme IV, step B: In a 2-Liter round bottomed flask fitted with a stirrer, a solution of cis-[(methylethyl)sulfonyl](2-phenylcyclopentyl)amine (10.37 g, 38.78 mmol) in trifluoroacetic acid (260 mL) is treated with sodium nitrate (9.9 g, 116.35 mmol) and the mixture is stirred at room temperature for five hours. The reaction mixture then quenched with H₂O (200 mL) and the mixture is extracted with CH₂Cl₂ (2×200 mL). The combined organic extracts are washed with saturated sodium bicarbonate (200 mL), dried over magnesium sulfate, filtered, and concentrated under vacuum to yield 14.3 g of crude product as a brown oil. This material is further purified by Prep HPLC 2000 (Hexane:EtOAc, 3:1) to provide the intermediate title compound (7.8 g, 64%) as a yellow solid.

### Preparation of cis-[2-(4-aminophenyl)cyclopentyl][(methylethyl)sulfonyl]amine

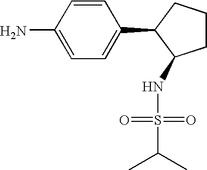

Scheme IV, step C: A solution of cis-[(methylethyl)sulfonyl][2-(4-nitrophenyl)cyclopentyl]amine (7.8 g, 24.97 mmol) and palladium on carbon (390 mg, 5 mole %) in absolute ethanol (200 mL) is combined in a Parr bottle. The mixture is shaken on a Parr shaker at room temperature under 40 psi of hydrogen for 12 hours. The reaction mixture is filtered through the Celite® and the filtrate is concentrated under vacuum to yield 5.9 g of brown crystals. This material is further purified by Prep HPLC 2000 (Hexanes:EtOAc, 1:1) to provide the intermediate title compound (3.4 g, 48%) as white crystals. EMS 284.0 (M*+1).

### Preparation of cis-[(methylethyl)sulfonyl](2-{4-[benzylamino]phenyl}cyclopentyl)amine

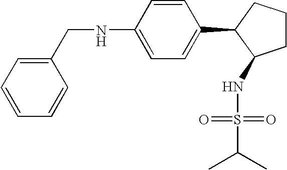

Scheme IV, step D: A solution of benzaldehyde (1.22 mL, 12.0 mmol) in acetic acid (36 mg, 0.6 mmol) is added to a solution of cis-[2-(4-aminophenyl)cyclopentyl][(methylethyl)sulfonyl]amine (3.4 g, 12.0 mmol) in methanol (48 mL). The reaction is stirred for four hours, then sodium borohydride (910 mg, 24 mmol) is added, and the mixture is stirred overnight at room temperature. The reaction mixture is then diluted with H₂O (150 mL), and the mixture is extracted with CH₂Cl₂ (3×100 mL). The combined organic layers are dried over K₂CO₃, filtered, and concentrated under vacuum to yield 4.36 g as a brown oil. This material is further purified by Prep HPLC 2000 (Hexanes:EtOAc, 3:1) to provide the intermediate title compound (2.9 g, 65%) as a yellow oil. EMS 373.0 (M*+1).

### Preparation of cis-2-{4-(2-{[(methylethyl)sulfonyl]amino}cyclopentyl)phenyl]benzylamino}ethanenitrile

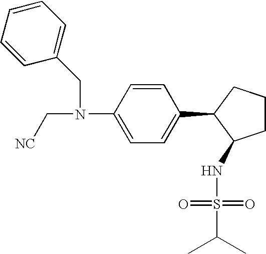

Scheme IV, step E: In a 250 mL 3-neck round bottom flask fitted with a thermometer and stirbar, a solution of sodium cyanide (401 mg, 8.17 mmol) in H₂O (20 mL) is treated with a solution of cis-[(methylethyl)sulfonyl](2-{4-[benzylamino]phenyl}cyclopentyl)amine (2.9 g, 7.79 mmol) in methanol (20 mL). The reaction mixture is cooled to 0° C. in an ice bath, and hydrochloric acid (2.32 mL) is added by syringe followed by formaldehyde, 37%, (0.23 mL, 8.17 mmol). The mixture is stirred for an additional three hours at 0° C. and gradually warmed to room temperature overnight. The reaction mixture is then quenched with H₂O (100 mL) and the mixture is extracted with CH₂Cl₂ (3×100 mL). The combined organic extracts are washed with brine (1×100 mL), dried over magnesium sulfate, filtered, and concentrated under vacuum to yield 1.95 g as a brown foam. This material is further purified by Prep HPLC 2000 (Hexanes:EtOAc, 3:1) to provide the intermediate title compound (850 mg, 27%) as a colorless foam.

### Preparation of cis-(2-{4-[(2-aminoethyl)benzylamino]phenyl}cyclopentyl)[(methylethyl)sulfonyl]amine

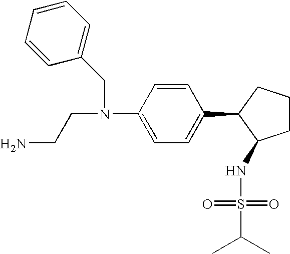

Scheme IV, step F: A solution of cis-2-{[4-(2-{[(methylethyl)sulfonyl]amino}cyclopentyl)phenyl]benzylamino}ethanenitrile (760 mg, 1.85 mmol) in THF (6.2 mL) is treated with a solution of borane-tetrahydrofuran 1M (1.85 mL, 1.85 mmol) and the mixture is heated at reflux (65° C.) overnight. The reaction mixture is cooled to room temperature and treated with a 1:1 mixture THF:MeOH (5.6 mL). When foaming ceases sodium hydroxide 5N (16.7 mL) is added to the reaction and the reaction mixture is heated at reflux (55° C.) for 5 hours. The mixture is then cooled to room temperature and the mixture is extracted with CH₂Cl₂ (2×50 mL). The combined organic layers are further washed by saturated sodium bicarbonate (50 mL), dried over magnesium sulfate, filtered, and concentrated under vacuum to yield 740 mg of a brown oil. This material is further purified on a Chromatotron® (CH₂Cl₂:MeOH, 9:1 with concentrated ammonium hydroxide (1 mL per 500 mL) to provide the intermediate title compound (370 mg, 48%) as a brown foam.

EMS 416.0 (M*+1)

Elemental Analysis:

| | | | |
|---|---|---|---|
| Theory: | C; 66.47 | H; 8.00 | N; 10.11 |
| Found: | C; 65.22 | H; 8.07 | N; 9.72 |

### Preparation of cis-[(methylethyl)sulfonyl](2-{[4-(2-{[(methylethyl)sulfonyl]amino}cyclopentyl)phenyl]benzylamino}ethyl)amine

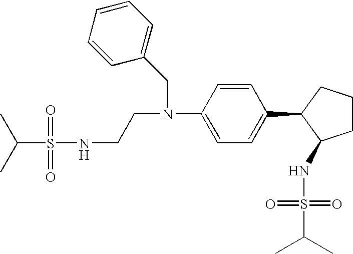

Scheme IV, step G: A solution of cis-(2-{4-[(2-aminoethyl)benzylamino]phenyl}cyclopentyl)[(methylethyl)sulfonyl]amine (175 mg, 0.4211 mmol) in CH₂Cl₂ (2.8 mL) and DBU (0.23 mL, 1.053 mmol) is treated with isopropylsulfonyl chloride (0.07 mL, 0.6317 mmol) at 0° C. The reaction mixture is warmed to room temperature while stirring overnight. The mixture is then quenched with H₂O (5 mL) and the mixture is extracted with CH₂Cl₂ (2×10 mL). The combined organic layers are washed with brine (10 mL), dried over magnesium sulfate, filtered, and concentrated under vacuum to yield 271 mg of a brown oil. This crude material is further purified on a Chromatotron® (Hexanes:EtOAc, 2:1) to provide the intermediate title compound (195 mg, 89%) as a brown foam. EMS 523.0 (M*+1).

Elemental Analysis:

| | | | |
|---|---|---|---|
| Theory: | C; 59.85 | H; 7.53 | N; 8.05 |
| Found: | C; 57.62 | H; 7.12 | N; 7.84 |

### Preparation of Final Title Compound

Scheme IV, step H: In a round bottom flask, a solution of cis-[(methylethyl)sulfonyl](2-{[4-(2-{([(methylethyl)sulfonyl]amino}cyclopentyl)phenyl]benzylamino}ethyl)amine (80 mg, 0.1533 mmol) in THF (10 mL) is treated with ammonium formate (48 mg, 0.7667 mmol) and palladium on carbon (10 mg). The reaction mixture is stirred at room temperature overnight and then heated at reflux (65° C.) for 8 hours. The mixture is then filtered through Celite®, and the Celite® cake is washed with H₂O (10 mL). Ethyl acetate is added to the filtrate and the mixture is extracted with ethyl acetate (3×10 mL). The combined organic layers are washed with brine (40 mL), dried over magnesium sulfate, filtered, and concentrated under vacuum to yield 104 mg as a purple foam. This crude material is further purified on a Chromatotron® (Hexanes:EtOAc;1:1) to provide the final title compound (6 mg, 9%) as a yellow oil. EMS 433.0 (M*+1). FI-EMS 432 (M*+1)

### EXAMPLE 13

### Preparation of cis-(2-{[4-(2-{[(methylethyl)sulfonyl]amino}cyclopentyl)phenyl]amino}ethyl)(methylsulfonyl)amine

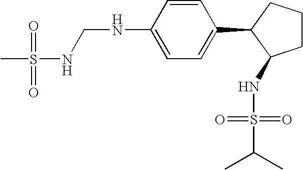

### Preparation of cis-(2-{[4-(2-{[(methylethyl)sulfonyl]amino}cyclopentyl)phenyl]benzylamino}ethyl)(methylsulfonyl)amine

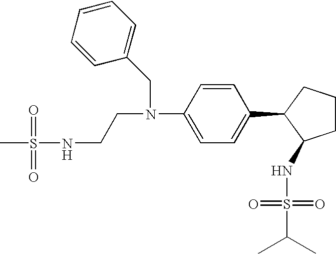

Scheme IV, step G: In a round bottom flask, a solution of cis-(2-{4-[(2-aminoethyl)benzylamino]phenyl}cyclopentyl)[(methylethyl)sulfonyl]amine (175 mg, 0.4211 mmol, prepared in example 12) in CH₂Cl₂ (3 mL) is combined with DBU (0.23 mL, 1.053 mmol) at 0° C. and treated with isopropylsulfonyl chloride (0.07 mL, 0.6317 mmol). The mixture is then gradually warmed to room temperature overnight. The reaction is then quenched with H₂O (5 mL), and mixture is extracted with CH₂Cl₂ (2×10 mL). The combined organic extracts are washed with brine (10 mL), dried over magnesium sulfate, filtered, and concentrated under vacuum to yield 292 mg of a brown oil. This crude material is further purified on a Chromatotron® (Hexanes:EtOAc, 1:1) to provide the is intermediate title compound (149 mg, 72%) as a yellow foam. EMS 495.0 (M*+1).

Elemental Analysis:

| | | | |
|---|---|---|---|
| Theory: | C; 58.39 | H; 7.15 | N; 8.51 |
| Found: | C; 56.50 | H; 6.75 | N; 8.20 |

### Preparation of Final Title Compound

Scheme IV, step H: In a round bottom flask, a solution of cis-(2-{[4-(2-{[(methylethyl)sulfonyl]amino}cyclopentyl)phenyl]benzylamino}ethyl)(methylsulfonyl)amine (65 mg, 0.132 mmol) in THF (10 mL) is treated with ammonium formate (42 mg, 0.66 mmol) and palladium on carbon (10 mg). The reaction mixture is stirred at room temperature overnight and then heated at reflux (65° C.) for 8 hours. The mixture is then filtered through Celite®, and the Celite® cake is washed with H₂O (10 mL). Ethyl acetate is added to the filtrate and mixture is extracted with ethyl acetate (3×10 mL). The combined organic layers are washed with brine (40 mL), dried over magnesium sulfate, filtered, and concentrated under vacuum to yield 59 mg of a purple foam. This crude material is further purified on a Chromatotron® (Hexanes:EtOAc;1:1) to provide the final title compound (6 mg, 11%) as a yellow oil.

EMS 405.0 (M*+1) FI-EMS 404 (M*+1)

### EXAMPLE 14

### Preparation of {2-hydroxy-2-[4-({2-[(methylsulfonyl)amino]ethyl}benzylamino)phenyl]propyl}[(methylethyl)sulfonyl]amine

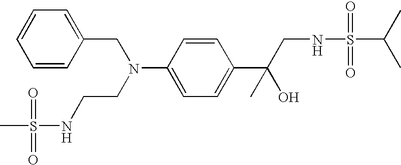

### Preparation of 1-amino-2-(4-nitrophenyl)propan-2-ol hydrochloride

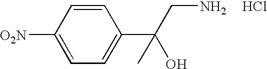

Scheme V, step A: Trimethylsylilcyanate (100 mL, 703.05 mmol, Aldrich) is added to 4-nitroacetophenone (38.75 g, 234.35 mmol) and zinc iodide (7.5 g, 23.44 mmol) in a 1 L round bottomed flask, neat, fitted with a stirbar, and under a nitrogen atmosphere, at room temperature. The mixture is stirred overnight and diluted with 100 mL dichloromethane, followed by slow addition of 100 mL saturated sodium carbonate. The layers are separated, and the organic layer is washed once with deionized water, filtered through magnesium sulfate (MgSO₄), and concentrated under vacuum, yielding 25.9 g orange oil. This product is dissolved in tetrahydrofuran (327 mL, anhydrous) in a 1 L 3-neck round bottomed flask, fitted with a thermometer, reflux condenser, stirbar, and addition funnel. Borane-dimethylsulfide, 2M, (98 mL, 195.96 mmol) is added dropwise, and the reaction is stirred overnight at room temperature. Concentrated hydrochloric acid (HCl) is added to pH=2, and the acidic mixture is poured into 1 L of diethyl ether (Et₂O), and the precipitate is vacuum filtered off. The precipitate is washed with Et₂O (2×100 mL). The precipitate is dried by heated vacuum for 2 hours at 45° C., to provide the intermediate title compound (11.6 g, 25%).

Mass Spec-Electrospray (MS-ES) 197.0 (M*+1)

### Preparation of [2-hydroxy-2-(4-nitrophenyl)propyl][(methylethyl)sulfonyl]amine

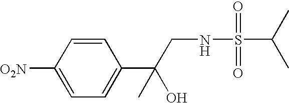

Scheme V, step B: 1-Amino-2-(4-nitrophenyl)propan-2-ol hydrochloride (11.6 g, 59.12 mmol), is added to THF (394 mL) in a 2-Liter, 3-neck, round bottomed flask fitted with a thermometer, addition funnel, and under a nitrogen atmosphere. Triethylamine (20.6 mL, 147.8 mmol) is added, and the reaction temperature is reduced to 0° C. in an ice bath. Isopropylsulfonyl chloride (10.0 mL, 88.68 mmol) is added by addition funnel at 0° C., and gradually warmed to room temperature overnight in the ice bath. The reaction is quenched with water (400 mL), and the layers are separated. The aqueous layer is extracted with CH₂Cl₂ (2×300 mL), and the organic extracts are combined. The combined organic extracts are washed with brine (1×500 mL), filtered through MgSO₄, and concentrated under vacuum, yielding 15.4 g brown oil. This crude material is further purified by running through two Waters Prep-pak's®, on a Waters Prep HPLC 2000, in a 1:1 hexanes:ethyl acetate solvent system to provide the intermediate title compound (1.41 g, 8%) as a yellow oil.

Mass Spec-Electrospray (MS-ES) 303.0 (M*+1).

### Preparation of [2-(4-aminophenyl)-2-hydroxypropyl][(methylethyl)sulfonyl]amine

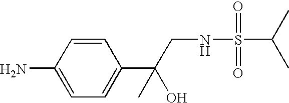

Scheme V, step C: [2-Hydroxy-2-(4-nitrophenyl)propyl][(methylethyl)sulfonyl]amine (2.41 g, 7.97 mmol) is dissolved into absolute ethanol (200 mL) and added to 5% palladium on carbon, wetted with ethanol (2 mL) in a nitrogen flushed Parr bottle. The reaction vessel is stopped off and shaken on a Parr shaker at room temperature under 40 psi of hydrogen, overnight. The reaction mixture is then filtered through Celite® to remove the catalyst, and the filtrate is concentrated under vacuum, yielding 1.82 g colorless oil. This material was further purified by running through one Waters Prep-pak's®, on a Waters Prep HPLC 2000, in a 1:1 hexanes:ethyl acetate solvent system to provide the intermediate title compound (1.1 g, 64%) white crystals. Electrospray-MS 274.0 (M*+1)

### Preparation of (2-hydroxy-2-{4-[benzylamino]phenyl}propyl)[(methylethyl)sulfonyl]amine

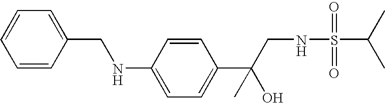

Scheme V, step D: A mixture of benzaldehyde (0.52 mL, 5.14 mmol) and acetic acid (15 mg, 0.257 mmol), is added to a solution of [2-(4-aminophenyl)-2-hydroxypropyl][(methylethyl)sulfonyl]amine (1.4 g, 5.14 mmol) in MeOH (23 mL), in a 250 mL round bottomed flask fitted with a stirbar, and under a nitrogen atmosphere. The reaction is stirred for four hours, then sodium borohydride is added, and stirred overnight, at room temperature. The reaction is then diluted with water (150 mL), and extracted with CH₂Cl₂ (3×100 mL). The organic extracts are combined and filtered through potassium carbonate (K₂CO₃), and concentrated under vacuum, yielding 2.132 g of a brown oil. This material is further purified by running it through one Waters Prep-pak®, on a Waters Prep HPLC 2000, in 3:2 hexanes:ethyl acetate solvent system, to provide the intermediate title compound (1.62 g, 87%) as a yellow oil.

Electrospray-MS 373.0 (M*+1).

### Preparation of 2-{[4-(1-hydroxy-1-methyl-2-{[(methylethyl)sulfonyl]amino}ethyl)phenyl]benzylamino}ethanenitrile

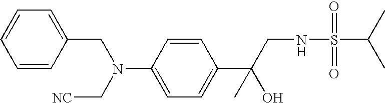

Scheme V, step E: A solution of (2-hydroxy-2-{4-[benzylamino]phenyl}propyl)[(methylethyl)sulfonyl]amine (1.0 g, 2.76 mmol) in MeOH (7 mL) is added to sodium cyanide (142 mg, 2.90 mmol) dissolved in H₂O (7 mL) in a 100 mL 3-neck round bottomed flask fitted with a thermometer, stirbar, and under a nitrogen atmosphere. The reaction vessel is cooled to 0° C. in an ice bath, and hydrochloric acid (0.8 mL) is added by syringe, followed by addition of formaldehyde, 37%, (0.2 mL, 2.90 mmol) by syringe, and the reaction mixture is stirred for an additional three hours at 0° C. The ice bath is removed and the reaction mixture is gradually warmed to room temperature overnight. The reaction is then poured into H₂O (50 mL) and extracted with CH₂Cl₂ (3×25 mL). The organic extracts are combined, washed with brine (1×100 mL), filtered through MgSO₄, and concentrated under vacuum, yielding 939 g of a brown foam. This material is further purified by running it over a 6000 μm rotor on a Chromatotron®, in 1:1 hexanes:ethyl acetate solvent system to provide the intermediate title compound (690 mg, 62%) as a colorless foam.

Electrospray-MS 402.0 (M*+1)

Elemental Analysis:

| | | | |
|---|---|---|---|
| C (Theory) | 62.82 | C (Found) | 62.42 |
| H (Theory) | 6.78 | H (Found) | 6.74 |
| N (Theory) | 10.47 | N (Found) | 10.14 |

### Preparation of (2-{4-[(2-aminoethyl)benzylamino]phenyl}-2-hydroxypropyl)[(methylethyl)sulfonyl]amine

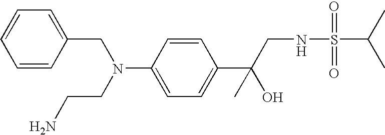

Scheme V, step F: A solution of 2-{[4-(1-hydroxy-1-methyl-2-{[(methylethyl)sulfonyl]amino}ethyl)phenyl]benzylamino}ethanenitrile (600 mg, 1.5 mmol) in warm THF (3 mL) is added to lithium aluminum hydride (57 mg, 1.5 mmol) in THF (7 mL) in a 100 mL 3 neck round bottomed flask, fitted with a thermometer, addition funnel, stirbar, under a nitrogen atmosphere, in an ice bath (0° C.). The reaction mixture is stirred overnight at room temperature. The reaction is then cooled in an ice bath (0° C.), and 1 mL H₂O, 1 mL 5N sodium hydroxide (NaOH), and 3 mL H₂O are added in consecutive increments. After foaming ceases, the mixture is filtered, and the precipitate is washed with 10 mL hot THF. The organic filtrate is concentrated under vacuum yielding 456 mg as a yellow oil. This material is further purified by running it over a 4000 μm rotor on a Chromatotron®, in 9:1 CH₂Cl₂:methanol solvent system, to provide the intermediate title compound (80 mg, 13%) as a yellow foam.

Electrospray-MS 406.0 (M*+1)

### Preparation of Final Title Compound

Scheme V, step G: A solution of (2-{4-[(2-aminoethyl)benzylamino]phenyl}-2-hydroxypropyl)[(methylethyl)sulfonyl]amine (80 mg, 0.1973 mmol) in THF (1.3 mL) is treated with DBU (0.08 mL, 0.4933 mmol), and the reaction temperature is reduced to 0° C. with an ice bath. Methanesulfonyl chloride (0.02 mL, 0.1973 mmol) is added by syringe at 0° C., and gradually warmed to room temperature overnight in the ice bath. The reaction is then quenched with H₂O (2 mL), and the layers are separated. The aqueous layer is then extracted with CH₂Cl₂ (2×5 mL), the organic extracts are combined, washed with brine (1×10 mL), filtered through MgSO₄, and concentrated under vacuum, yielding 100 mg of a yellow oil. This material was further purified by running it over a 1000 μm rotor on a Chromatotron®, in a 3:1 CH₂Cl₂:ethyl acetate solvent system to provide the final title compound (7 mg, 7%) as a yellow oil.

Electrospray-MS 485.0 (M*+1)

### EXAMPLE 15

### Preparation of (methylsulfonyl)[2-(6-{2-[(methylsulfonyl)amino]ethoxy}(2-naphthyl))ethyl]amine

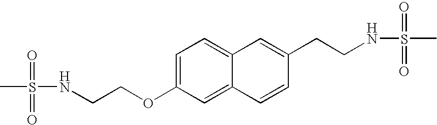

### Preparation of (6-methoxy-2-naphthyl)methan-1-ol

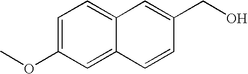

A solution of 6-methoxy-2-naphthoic acid (15.0 g, 74.2 mmol) in warm THF (75 mL) is added to lithium aluminum hydride (2.83 g, 74.2 mmol) in THF (150 mL), in a 1 L 3-neck round bottomed flask, fitted with a thermometer, stir bar, and addition funnel, in an ice bath (0° C.), under a nitrogen atmosphere. The reaction is stirred overnight at room temperature: It is then cooled in ice bath (0° C.), with consecutive addition of 1 mL increments of deionized water and 5N sodium hydroxide (NaOH). 3 mL deionized water is then added (as per Feiser work up). The precipitate is filtered off and washed with hot THF (75 mL). The filtrate is concentrated under vacuum yielding the intermediate title compound (9.23 g, 66%) as a white solid.

### Preparation of 6-(bromomethyl)-2-methoxynaphthalene

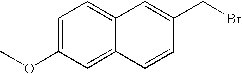

Bromine (2.82 mL, 55.04 mmol) is added dropwise to triphenylphosphine (9.4 g, 35.78 mmol) dissolved in methylene chloride (12 mL) in a 250 mL 3-neck round bottomed flask, fitted with a thermometer, stirbar, addition funnel, under a nitrogen atmosphere, and in an ice bath (0° C.). The solution turns a yellow color and is retitrated with triphenylphosphine until a white color emerges. (6-Methoxy-2-naphthyl)methan-1-ol (5.18 g, 27.52 mmol) dissolved in 1:1 THF:methylene chloride (32 mL) is then added, the ice bath is removed, and the reaction mixture is stirred to room temperature. The reaction mixture is then concentrated under vacuum and extracted with diethyl ether (3×20 mL), and the combined extracts are concentrated under vacuum to yield 16.60 g of a yellow oil. This material is purified by silica gel chromatography, using a Waters HPLC Prep 2000, over two Prep-Pak, in a 3:1 hexanes:ethyl acetate solvent system to yield the intermediate title compound (3.923 g, 57%). Electrospray-MS M*±1 (252.1, 250.1)

### Preparation of 2-(6-methoxy-2-naphthyl)ethanenitrile

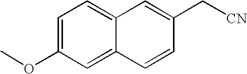

A solution of 6-(bromomethyl)-2-methoxynaphthalene (3.923 g, 15.62 mmol) in dimethylsulfoxide (10 mL) is added to a solution of sodium cyanide (1.15 g, 23.43 mmol) in DMSO (15 mL), heated to 50° C., in a 250 mL 3-neck round bottomed flask, fitted with a stirbar, thermometer, condenser, and addition funnel, under a nitrogen atmosphere. The reaction mixture is stirred at 50° C. for 0.5 hr, then the temperature is increased to 70° C., and stirred an additional 1.0 hr. The reaction is then cooled to room temperature and poured over ice. The mixture is then extracted with methylene chloride (3×20 mL), and the organic extracts are washed with deionized water (3×50 mL). The organic extracts are then filtered through magnesium sulfate, and concentrated under vacuum, yielding 2.10 g of a brown oil. This material is purified by silica gel chromatography, using a Waters HPLC Prep 2000, over two Prep-Pak®, in a 3:1 hexanes:ethyl acetate solvent system to provide the intermediate title compound (990 mg, 32%) as yellow powder. Electrospray-MS 198.1 (M*+1)

### Preparation of 2-(6-methoxy-2-naphthyl)ethylamine

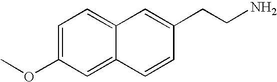

2-(6-Methoxy-2-naphthyl)ethanenitrile (5.0 g, 25.35 mmol) is dissolved into absolute ethanol (200 mL), saturated with ammonia, and added to Rainey nickel (2.5 g) wetted with ethanol (2 mL), in a nitrogen flushed Parr bottle. The reaction vessel is stopped off and shaken on a Parr shaker at 50° C. and 60 psi hydrogen overnight. The catalyst is then filtered off with Celite®, and the organic layer is concentrated under vacuum yielding 6.1 g greenish oil. This material is further purified by running through one Waters Prep-pak's®, on a Waters Prep HPLC 2000, in a 1:1 methylene chloride:ethyl acetate solvent system to provide the intermediate title compound (5.70 g) as white crystals. Electrospray-MS 202.0 (M*+1)

### Preparation of [2-(6-methoxy(2-naphthyl))ethyl](methylsulfonyl)amine

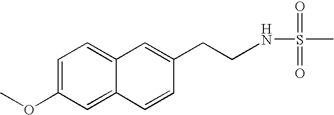

A solution of 2-(6-methoxy-2-naphthyl)ethylamine (2.85 g, 14.16 mmol) in THF (95 mL) in a 250 mL 3-neck round bottomed flask fitted with a thermometer, addition funnel, and under a nitrogen atmosphere is treated with triethylamine (3.94 mL, 28.32 mmol), and the reaction temperature is reduced to 0° C. in an ice bath. Methanesulfonyl chloride (2.4 mL, 21.24 mmol) is added by addition funnel at 0° C., and the mixture is gradually warmed to room temperature overnight. The reaction is then quenched with deionized water (H₂O, 100 mL), and the layers are separated. The aqueous layer is extracted with methylene chloride (2×75 mL), and the organic layers are combined. The combined organic layers are washed with brine (1×100 mL), filtered through MgSO₄, and concentrated under vacuum, yielding 3.10 g orange oil. This crude material is further purified by running through one Waters Prep-Pak®, on a Waters Prep HPLC 2000, in a 3:1 hexanes:ethyl acetate solvent system, to provide the intermediate title compound (600 mg, 14%) as a yellow solid. Mass Spec-Electrospray (MS-ES) 308.0 (M*+1)

### Preparation of [2-(6-hydroxy(2-naphthyl))ethyl](methylsulfonyl)amine

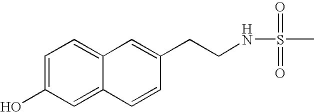

A solution of boron tribromide (3.6 mL, 3.0 eq) in methylene chloride (15 mL) is added dropwise to [2-(6-methoxy(2-naphthyl))ethyl](methylsulfonyl)amine (12.76 mmol) in methylene chloride (70 mL) in a 250 mL round bottomed flask fitted with a stirbar, and under a nitrogen atmosphere, at room temperature. The reaction mixture is stirred for two hours, or until starting material is consumed as indicated by Thin Layer Chromatography. Water (60 mL) is added slowly, and the layers are separated. The organic layer is filtered through potassium carbonate, and concentrated under vacuum to provide the intermediate title compound which is used without further purification.

### Preparation of 2-(6-{2-[(methylsulfonyl)amino]ethyl}-2-naphthyloxy)ethanenitrile

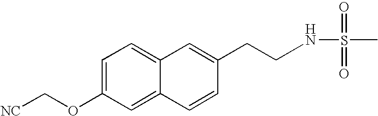

A solution of [2-(6-hydroxy(2-naphthyl))ethyl](methylsulfonyl)amine (12.36 mmol) in acetone (82 mL) is treated with cyanomethylbromide (0.95 mL, 1.1 eq) and potassium carbonate (4.27 g, 2.5 eq) in a 250 mL round bottomed flask fitted with a stirbar and under a nitrogen atmosphere. The reaction mixture is stirred at room temperature, overnight. The reaction mixture is then washed with water (80 mL) and extracted with methylene chloride (3×50 mL). The organic layers are combined and filtered through MgSO₄, and concentrated under vacuum. This material is further purified by silica gel chromatography, employing the Water's Prep 2000 while eluting with a solvent of hexanes/ethyl acetate 3:1 to provide the title compound.

### Preparation of {2-[6-(2-aminoethoxy)(2-naphthyl)]ethyl}(methylsulfonyl)amine

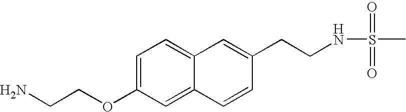

2-(6-{2-[(Methylsulfonyl)amino]ethyl}-2-naphthyloxy)ethanenitrile (191.91 mmol), borane dimethylsulfide, reagent (192 mL, 10 M, 10.0 equivalents), and THF (2300 mL) are combined in a 3000 mL 3-neck round bottomed flask, affixed with a thermometer, condenser, and rubber stopper. Under a nitrogen atmosphere, with stirring, reaction is refluxed at 70° C. overnight. The reaction mixture is cooled to room temperature. 1:1 THF:MeOH (581 mL) is added by syringe, and when foaming ceases, 5N NaOH (1745 mL) is added by syringe. Then the mixture is refluxed at 55° C. for an additional five hours. The reaction mixture is cooled to room temperature, and extracted three times with methylene chloride (3×1000 mL). The organic layer is dried with sodium sulfate, filtered, and concentrated in vacuum. This material is washed with a saturated sodium bicarbonate solution (200 mL) for two hours, and extracted three times with methylene chloride (3×200 mL). The organic layer is dried with sodium sulfate, filtered, and concentrated in vacuum to provide the intermediate title compound which is used without further purification.

### Preparation of Final Title Compound

Scheme II, step D: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The reaction mixture is cooled to 0° C., and triethylamine (0.2 mL, 2.5 equivalents) is added by syringe, followed by methanesulfonyl chloride (0.07 mL, 1.5 Eq.), also by syringe, and mixed overnight. The reaction is quenched with 20 mL water, and layers are separated. The organic layer is washed with water (20 mL), dried with sodium sulfate, filtered, and concentrated in vacuum. The crude residue is further purified by silica gel chromatography on a Chromatotron® employing a 2000 uM rotor in a 1:1 hexanes:ethyl acetate solvent system to provide the final title compound.

### EXAMPLE 16

### Preparation of [(methylethyl)sulfonyl][2-(6-{2-[(methylsulfonyl)amino]ethoxy}2-naphthyl))ethyl]amine

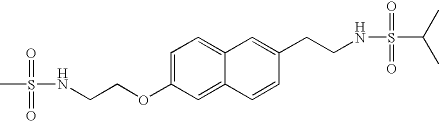

### Preparation of [2-(6-methoxy(2-naphthyl))ethyl][(methylethyl)sulfonyl]amine

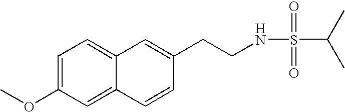

2-(6-Methoxy-2-naphthyl)ethylamine (2.85 g, 14.16 mmol) is added to THF (95 mL) in a 250 mL 3-neck round bottomed flask fitted with a thermometer, addition funnel, and under a nitrogen atmosphere. Triethylamine (3.94 mL, 28.32 mmol) is added, and the reaction temperature is reduced to 0° C. in an ice bath. Isopropylsulfonyl chloride (1.64 mL, 21.24 mmol) is added by addition funnel at 0° C., and gradually warmed to room temperature overnight. The reaction is quenched with deionized water (100 mL), and the layers are separated. The aqueous layer is extracted with methylene chloride (2×75 mL), and the organic layers are combined, washed with brine (1×100 mL), filtered through MgSO₄, and concentrated under vacuum, yielding 1.58 g orange oil. This crude material is further purified by running through one Waters Prep-Pak®, on a Waters Prep HPLC 2000, in a 3:1 hexanes:ethyl acetate solvent system, to provide the intermediate title compound (560 mg, 14%) as a yellow solid. Mass Spec-Electrospray (MS-ES) 280.0 (M*+1)

### Preparation of [2-(6-hydroxy(2-naphthyl))ethyl][(methylethyl)sulfonyl]amine

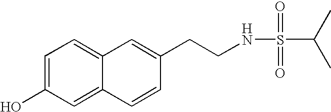

A solution of boron tribromide (13 mL, 3.0 eq) in methylene chloride (15 mL) is added dropwise to [2-(6-methoxy(2-naphthyl))ethyl][(methylethyl)sulfonyl]amine (45.73 mmol) in methylene chloride (260 mL) in a 1 L round bottomed flask fitted with a stirbar, and under a nitrogen atmosphere, at room temperature. The reaction mixture is stirred for two hours, or until starting material is consumed as indicated by Thin Layer Chromatography (TLC). Water (60 mL) is added slowly, and then the layers are separated. The organic layer is filtered through potassium carbonate, and concentrated under vacuum to provide the intermediate title compound which is used without further purification.

### Preparation of 2-[6-(2-{[(methylethyl)sulfonyl]amino}ethyl)-2-naphthyloxy]ethanenitrile

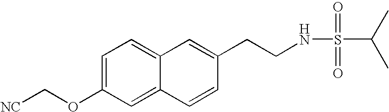

[2-(6-Hydroxy(2-naphthyl))ethyl][(methylethyl)sulfonyl]amine (13.07 mmol), cyanomethylbromide (1.00 mL, 1.1 eq), potassium carbonate (4.52 g, 2.5 eq), and acetone (87 mL) are combined in a 250 mL round bottomed flask fitted with a stirbar and under a nitrogen atmosphere. The reaction mixture is stirred at room temperature, overnight. The reaction mixture is then washed with water (80 mL) and extracted with methylene chloride (3×50 mL). The organic layers are combined and filtered through MgSO₄, and concentrated under vacuum. This material is further purified by silica gel chromatography, employing the Water's Prep 2000 while eluting with a solvent of hexanes/ethyl acetate 3:1 to yield the intermediate title compound.

### Preparation of {2-[6-(2-aminoethoxy)(2-naphthyl)]ethyl}[(methylethyl)sulfonyl]amine

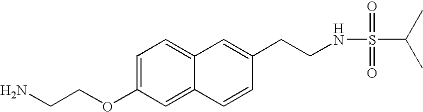

2-[6-(2-{[(Methylethyl)sulfonyl]amino}ethyl)-2-naphthyloxy]ethanenitrile (5.66 mmol), borane dimethylsulfide reagent (2.27 mL, 10 M, 10.0 equivalents), and THF (68 mL) are combined in a 250 mL 3-neck round bottomed flask, affixed with a thermometer, condenser, and rubber stopper. Under a nitrogen atmosphere, with stirring, reaction is refluxed at 70° C. overnight. The reaction mixture is cooled to room temperature. 1:1 THF:MeOH (17 mL) is added by syringe, and when foaming ceases, 5N NaOH (51 mL) is added by syringe. Then the mixture is refluxed at 55° C. for an additional five hours. The reaction mixture is cooled to room temperature, and extracted three times with methylene chloride (3×50 mL). The organic layer is dried with sodium sulfate, filtered, and concentrated in vacuum. This material is washed with a saturated sodium bicarbonate solution (50 mL) for two hours, and extracted three times with methylene chloride (3×50 mL). The organic layer is dried with sodium sulfate, filtered, and concentrated in vacuum to provide the intermediate title compound which is used without further purification.

### Preparation of Final Title Compound

Scheme II, step D: {2-[6-(2-Aminoethoxy)(2-naphthyl)]ethyl}[(methylethyl)sulfonyl]amine (0.571 mmol) is dissolved in methylene chloride in a 15 mL round bottomed flask, under a nitrogen system. The reaction mixture is cooled to 0° C., and triethylamine (0.2 mL, 2.5 eq) is added by syringe, followed by methanesulfonyl chloride (0.07 mL, 1.5 Eq.), also by syringe, and mixed overnight. The reaction is quenched with 20 mL water, and the layers are separated. The organic layer is washed with water (20 mL), dried with sodium sulfate, filtered, and concentrated in vacuum. This material is further purified by silica gel chromatography on a Chromatotron® employing a 2000 uM rotor in a 1:1 hexanes:ethyl acetate solvent system to provide the final title compound.

### EXAMPLE 17

### Preparation of {2-[6-(2-{[(methylethyl)sulfonyl]amino}ethoxy)(2-naphthyl)]ethyl}(methylsulfonyl)amine

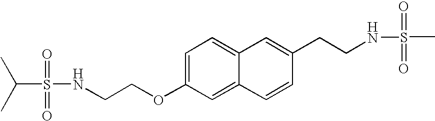

Scheme II, step D: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The reaction mixture is cooled to 0° C., and triethylamine (0.2 mL, 2.5 eq.) is added by syringe, followed by isopropylsulfonyl chloride (0.095 mL, 1.5 eq.), also by syringe, and mixed overnight. The reaction is quenched with 20 mL water, and the layers are separated. The organic layer is washed with water (20 mL), dried with sodium sulfate, filtered, and concentrated in vacuum. This material is further purified by silica gel chromatography on a Chromatotron® employing a 2000 uM rotor in a 1:1 hexanes:ethyl acetate solvent system to provide the title compound.

### EXAMPLE 18

### Preparation of [(methylethyl)sulfonyl]{2-[6-(2-{[(methylethyl)sulfonyl]amino}ethoxy)(2-naphthyl]ethyl}amine

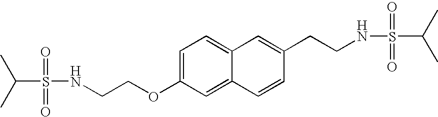

Scheme II, step D: {2-[6-(2-Aminoethoxy)(2-naphthyl)]ethyl}[(methylethyl)sulfonyl]amine (0.571 mmol) is dissolved in methylene chloride in a 15 mL round bottomed flask, under a nitrogen system. The reaction mixture is cooled to 0° C., and triethylamine (0.2 mL, 2.5 eq.) is added by syringe, followed by isopropylsulfonyl chloride (0.095 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The crude residue is further purified by silica gel chromatography on a Chromatotron® employing a 2000 uM rotor in a 1:1 hexanes:ethyl acetate solvent system to provide the title compound.

### EXAMPLE 19

### Preparation of [(methylethyl)sulfonyl][2-(6-{2-[(methylsulfonyl)amino]ethoxy}(2-naphthyl))propyl]amine

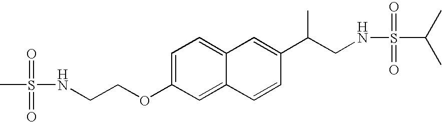

### Preparation of 2-(6-methoxy-2-naphthyl)propanamide

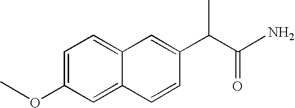

A solution of 2-(6-methoxy-2-naphthyl)propionic acid (50.0 g, 217.14 mmol, LKT Laboratories, Inc., St. Paul Minn.) in THF (217 mL) is added dropwise to oxalyl chloride (76 mL, 868.56 mmol) in THF (217 mL), in a 2000 mL round bottomed flask fitted with a stirbar and under a nitrogen atmosphere. The reaction is catalyzed with dimethylformamide (1 drop), and stirred at room temperature for two hours. The reaction mixture is then concentrated under vacuum, and diluted with 1,4-dioxane (217 mL). This solution is added dropwise to concentrated ammonium hydroxide (65 mL) and stirred overnight. The reaction mixture is extracted with ethyl acetate (2×250 mL), the organic extracts are combined, filtered through potassium carbonate, and concentrated under vacuum, yielding 91.2 g of an off-white solid. This material is purified by vacuuming off excess solvent to provide the intermediate title compound (44.0 g, 88%) as a brown solid._Electrospray-MS 230.1 (M*+1).

### Preparation of 2-(6-methoxy-2-naphthyl)propylamine

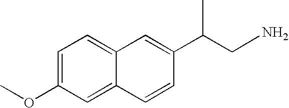

2-(6-Methoxy-2-naphthyl)propanamide (44.0 g, 191.91 mmol), borane dimethylsulfide reagent (192 mL, 10 M), and THF (2312 mL) are combined in a 3000 mL 3-neck round bottomed flask, affixed with a thermometer, condenser, and addition funnel. Under a nitrogen atmosphere, with stirring, the reaction is refluxed at 70° C. overnight. The reaction mixture is cooled to room temperature. 1:1 THF:MeOH (17 mL) is then added by syringe, and when foaming ceases, 5N NaOH (51 mL) is added by syringe. Then the mixture is refluxed at 55° C. for an additional five hours. The reaction mixture is then cooled to room temperature, and extracted three times with methylene chloride (3×50 mL). The organic layer is dried with sodium sulfate, filtered, and concentrated in vacuum. This material is stirred with a saturated sodium bicarbonate solution (50 mL) for two hours, and extracted three times with methylene chloride (3×50 mL). The organic layer is dried with sodium sulfate, filtered, and concentrated in vacuum to provide the intermediate title compound (35.32 g, 85%) as a white solid. Electrospray-MS 216.0 (M*+1).

### Preparation of [2-(6-methoxy(2-naphthyl))propyl][(methylethyl)sulfonyl]amine

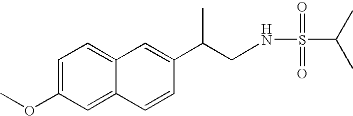

2-(6-Methoxy-2-naphthyl)propylamine (35.32 g, 164.05 mmol) is dissolved in methylene chloride in a 3000 mL round bottomed flask, under a nitrogen system. The mixture is cooled to 0° C., and triethylamine (68.5 mL, 492.15 mmol) is added by syringe, followed by isopropylsulfonyl chloride (36.85 mL, 328.1 mmol), also by syringe, and the reaction mixture is stirred overnight. The reaction is quenched with 1000 mL water, and the layers are separated. The organic layer is washed with water (1000 mL), dried with sodium sulfate, filtered, and concentrated in vacuum, yielding 69.1 g of a viscous yellow oil. This material is further purified by silica gel chromatography on a Waters prep 2000 employing two Prep-Pak's® in a 3:1 hexanes:ethyl acetate solvent system to yield the intermediate title compound (33.5 g, 64%) as white powder. Electrospray-M 322.0 (M*+1).

### Preparation of [2-(6-hydroxy(2-naphthyl))propyl][(methylethyl)sulfonyl]amine

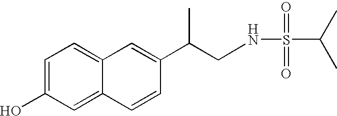

A solution of boron tribromide (3.6 mL, 38.28 mmol) in methylene chloride (15 mL), is added dropwise to [2-(6-methoxy(2-naphthyl))propyl][(methylethyl)sulfonyl]amine (12.76 mmol) in methylene chloride (70 mL) in a 250 mL round bottomed flask fitted with a stirbar, and under a nitrogen atmosphere, at room temperature. The reaction mixture is stirred for two hours, or until starting material is consumed as indicated by Thin Layer Chromatography (TLC). Water (60 mL) is added slowly, and then the layers are separated. The organic layer is filtered through potassium carbonate, and concentrated under vacuum to provide the intermediate title compound (3.80 g, 97%) as an off-white solid. Electrospray-MS 308.0 (M*+1).

### Preparation of 2-[6-(1-methyl-2-{[(methylethyl)sulfonyl]amino}ethyl)-2-naphthyloxy]ethanenitrile

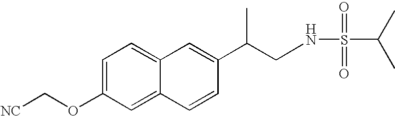

[2-(6-Hydroxy(2-naphthyl))propyl][(methylethyl)sulfonyl]amine (3.80 g, 12.36 mmol), cyanomethylbromide (0.95 mL, 13.60 mmol), and potassium carbonate (4.27 g, 30.9 mmol) are combined in acetone (82.4 mL), in a 250 mL round bottomed flask, at room temperature under a nitrogen atmosphere and stirred overnight. The reaction is washed with water (85 mL), and extracted with methylene chloride (3×100 mL). The organic extracts are combined and filtered through MgSO₄. The filtrate is concentrated under vacuum, yielding 3.97 g of brown oil. This material is purified by silica gel chromatography with a Waters Prep 2000, employing two Prep-Pak's® in a 3:1 hexanes:ethyl acetate solvent system, yielding the intermediate title compound (1.96 g, 46%) as a brown oil.

Electrospray-MS 347.0 (M*+1).

### Preparation of {2-[6-(2-aminoethoxy)(2-naphthyl)]propyl}[(methylethyl)sulfonyl]amine

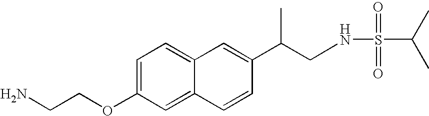

2-[6-(1-Methyl-2-{[(methylethyl)sulfonyl]amino}ethyl)-2-naphthyloxy]ethanenitrile (1.96 g, 5.66 mmol), borane dimethylsulfide reagent (2.27 mL, 22.64 mmol, 10 M), and THF (68 mL) are combined in a 250 mL 3-neck round bottomed flask, affixed with a thermometer, condenser, and rubber stopper. Under a nitrogen atmosphere, with stirring, the reaction is refluxed at 70° C. overnight. The reaction mixture is then cooled to room temperature. 1:1 THF:MeOH (17 mL) is added by syringe, and when foaming ceases, 5N NaOH (51 mL) is added by syringe. Then the mixture is refluxed at 55° C. for an additional five hours. The reaction mixture is cooled to room temperature, and extracted three times with methylene chloride (3×50 mL). The organic layer is dried with sodium sulfate, filtered, and concentrated in vacuum. This material is washed with a saturated sodium bicarbonate solution (50 mL) for two hours, and extracted three times with methylene chloride (3×50 mL). The organic layer is dried with sodium sulfate, filtered, and concentrated in vacuum, yielding 2.33 g of a brown oil. This material is purified by silica gel chromatography, with a Waters Prep 2000, employing one Prep-Pak's® in a 9:1 methylene chloride:methanol solvent system, to provide the intermediate title compound (1.17 g, 59%) as a brown foam. Electrospray-MS 351.0 (M*+1).

### Preparation of Final Title Compound

Scheme II, step D: {2-[6-(2-Aminoethoxy)(2-naphthyl)]propyl}[(methylethyl)sulfonyl]amine (0.571 mmol) is dissolved in methylene chloride in a 15 mL round bottomed flask, under a nitrogen system. The mixture is cooled to 0° C., and triethylamine (0.2 mL, 2.5 eq) is added by syringe, followed by methanesulfonyl chloride (0.07 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum, yielding 118 mg of a viscous yellow oil. This material is further purified by silica gel chromatography on a Chromatotron® employing a 2000 uM rotor in a 1:1 hexanes:ethyl acetate solvent system, to provide the final title compound (46 mg, 19%) as a brown foam.

Electrospray-MS 429.0 (M*+1).

Elemental Analysis:

| | | | |
|---|---|---|---|
| C(Theory): | 53.25 | C(Found): | 52.95 |
| H(Theory): | 6.59 | H(Found): | 6.68 |
| N(Theory): | 6.54 | N(Found): | 6.16 |

### EXAMPLE 20

### Preparation of [(methylethyl)sulfonyl]{2-[6-(2-{[(methylethyl)sulfonyl]amino}ethoxy)(2-naphthyl)]propyl}amine

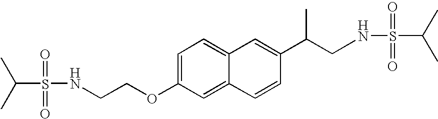

Scheme II, step D: {2-[6-(2-Aminoethoxy)(2-naphthyl)]propyl}[(methylethyl)sulfonyl]amine (0.571 mmol) is dissolved in methylene chloride in a 15 mL round bottomed flask, under a nitrogen system. The mixture is cooled to 0° C., and triethylamine (0.2 mL, 2.5 equivalents) is added by syringe, followed by isopropylsulfonyl chloride (0.095 mL, 1.5 eq.), also by syringe, and mixed overnight. The reaction is quenched with 20 mL water, and the layers are separated. The organic layer is washed with water (20 mL), dried with sodium sulfate, filtered, and concentrated in vacuum, yielding 326 mg of a viscous yellow oil. This material is further purified by silica gel chromatography on a Chromatotron® employing a 2000 uM rotor in a 1:1 hexanes:ethyl acetate solvent system to provide the title compound (100 mg, 38%) as a brown foam. Electrospray-MS 457.0 (M*+1).

Elemental Analysis:

| | | | |
|---|---|---|---|
| C(Theory): | 55.24 | C(Found): | 54.89 |
| H(Theory): | 7.06 | H(Found): | 7.00 |
| N(Theory): | 6.13 | N(Found): | 5.99 |

### EXAMPLE 21

### Preparation of (methylsulfonyl){2-[6-(2-{[trifluoromethyl)sulfonyl]amino}ethoxy)(2-naphthyl)]ethyl}amine

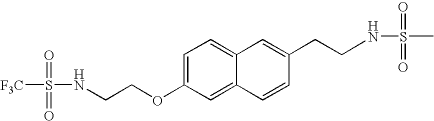

Scheme II, step D: {2-[6-(2-Aminoethoxy)(2-naphthyl)]ethyl}(methylsulfonyl)amine (0.571 mmol, prepared in example 15) is dissolved in methylene chloride in a 15 mL round bottomed flask, under a nitrogen system. The mixture is cooled to 0° C., and triethylamine (0.2 mL, 2.5 eq) is added by syringe, followed by trifluoromethylsulfonyl chloride (0.09 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the title compound.

### EXAMPLE 22

### Preparation of N-[2-(6-{2-[(methylsulfonyl)amino]ethyl}-2-naphthyloxy)ethyl]acetamide

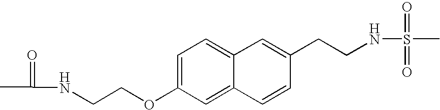

Scheme VII, step A: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 eq.) is added by syringe, followed by acetyl chloride (0.06 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the title compound.

### EXAMPLE 23

### Preparation of (methylsulfonyl)[2-(6-{2-[(phenylsulfonyl)amino]ethoxy}(2-naphthyl))ethyl]amine

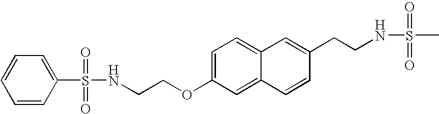

Scheme II, step D: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 eq.) is added by syringe, followed by phenylsulfonyl chloride (0.11 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the title compound.

### EXAMPLE 24

### Preparation of N-[2-(6-{2-[(methylsulfonyl)amino]ethyl}(2-naphthyloxy))ethyl]benzamide

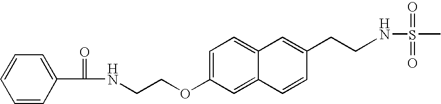

Scheme VII, step A: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 eq.) is added by syringe, followed by benzoyl chloride (0.04 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the final title compound.

### EXAMPLE 25

### Preparation of N-[2-(6-{2-[(methylsulfonyl)amino]ethyl}-2-naphthyloxy)ethyl]butanamide

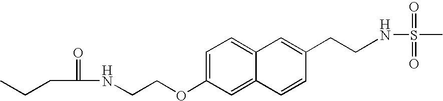

Scheme VII, step A: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 eq.) is added by syringe, followed by butyryl chloride (0.05 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the title compound.

### EXAMPLE 26

### Preparation of methoxy-N-[2-(6-{2-[(methylsulfonyl)amino]ethyl}(2-naphthyloxy))ethyl]carboxamide

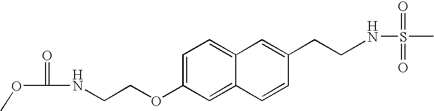

Scheme VII, step D: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 eq.) is added by syringe, followed by methyl chloroformate (0.06 mL, 1.5 eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate solvent system to provide the title compound.

### EXAMPLE 27

### Preparation of (methylphenylamino)-N-[2-(6-{2-[(methylsulfonyl)amino]ethyl}(2-naphthyloxy))ethyl]carboxamide

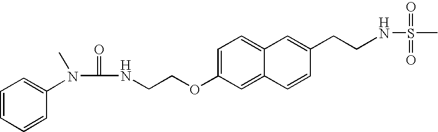

Scheme VII, step E: {2-[6-(2-Aminoethoxy)(2-naphthyl)]ethyl}(methylsulfonyl)amine (0.571 mmol) is dissolved in methylene chloride in a 15 mL round bottomed flask, under a nitrogen system. The mixture is cooled to 0° C., and triethylamine (0.2 mL, 2.5 equivalents) is added by syringe, followed by N-methyl-N-phenylcarbamoyl chloride (80 mg, 1.5 Eq.), also by syringe, and mixed overnight. The reaction is quenched with 20 mL water, and layers are separated. The organic layer is washed with water (20 mL), dried with sodium sulfate, filtered, and concentrated in vacuum. The residue is further purified by silica gel chromatography on a Chromatotron® employing a 2000 uM rotor in a 1:1 hexanes:ethyl acetate to provide the title compound.

### EXAMPLE 28

### Preparation of (methylsulfonyl){2-[6-(2-{[(trifluoromethyl)sulfonyl]amino}ethoxy)(2-naphthyl)]ethyl}amine

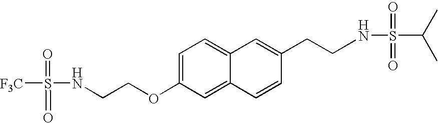

Scheme II, step D: The title compound is prepared in a manner analogous to the procedure set forth in example 21 from {2-[6-(2-aminoethoxy)(2-naphthyl)]ethyl}[(methylethyl)sulfonyl]amine (prepared in example 16).

### EXAMPLE 29

### Preparation of N-[2-(6-{2-[(methylsulfonyl)amino]ethyl}-2-naphthyloxy)ethyl]acetamide

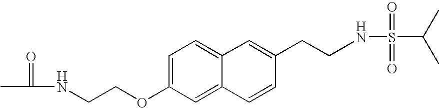

Scheme VII, step A: The title compound is prepared in a manner analogous to the procedure set forth in example 22 from {2-[6-(2-aminoethoxy)(2-naphthyl)]ethyl}[(methylethyl)sulfonyl]amine (prepared in example 16).

### EXAMPLE 30

### Preparation of (methylsulfonyl)[2-(6-{2-[(phenylsulfonyl)amino]ethoxy}(2-naphthyl))ethyl]amine

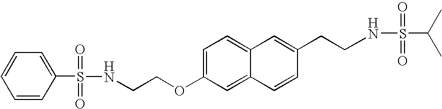

Scheme II, step D: The title compound is prepared in a manner analogous to the procedure set forth in example 23 from {2-[6-(2-aminoethoxy)(2-naphthyl)]ethyl}[(methylethyl)sulfonyl]amine (prepared in example 16).

### EXAMPLE 31

### Preparation of N-[2-(6-{2-[(methylsulfonyl)amino]ethyl}(2-naphthyloxy))ethyl]benzamide

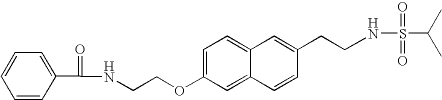

Scheme VII, step A: The title compound is prepared in a manner analogous to the procedure set forth in example 24 from {2-[6-(2-aminoethoxy)(2-naphthyl)]ethyl}[(methylethyl)sulfonyl]amine (prepared in example 16).

### EXAMPLE 32

### Preparation of N-[2-(6-{2-[(methylsulfonyl)amino]ethyl}-2-naphthyloxy)ethyl]butanamide

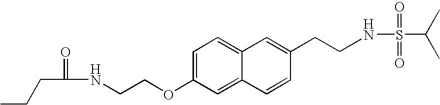

Scheme VII, step A: The title compound is prepared in a manner analogous to the procedure set forth in example 25 from {2-[6-(2-aminoethoxy)(2-naphthyl)]ethyl}[(methylethyl)sulfonyl]amine (prepared in example 16).

### EXAMPLE 33

### Preparation of methoxy-N-[2-(6-{2-[(methylsulfonyl)amino]ethyl}(2-naphthyloxy))ethyl]carboxamide

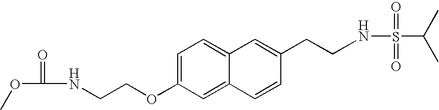

Scheme VII, step A: The title compound is prepared in a manner analogous to the procedure set forth in example 26 from {2-[6-(2-aminoethoxy)(2-naphthyl)]ethyl}[(methylethyl)sulfonyl]amine (prepared in example 16).

### EXAMPLE 34

### Preparation of (methylphenylamino)-N-[2-(6-{2-[(methylsulfonyl)amino]ethyl}(2-naphthyloxy))ethyl]carboxamide

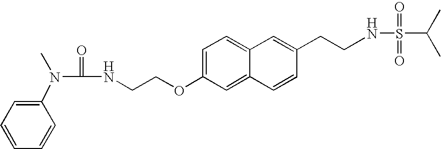

Scheme VII, step E: The title compound is prepared in a manner analogous to the procedure set forth in example 27 from {2-[6-(2-aminoethoxy)(2-naphthyl)]ethyl}[(methylethyl)sulfonyl]amine (prepared in example 16).

### EXAMPLE 35

### Preparation of [(methylethyl)sulfonyl]{2-[6-(2-{[(trifluoromethyl)sulfonyl]amino}ethoxy)(2-naphthyl)]propyl}amine

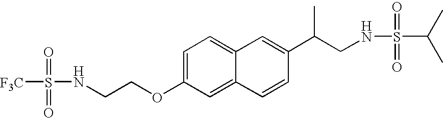

Scheme II, step D: The title compound is prepared in a manner analogous to the procedure set forth in example 21 from {2-[6-(2-aminoethoxy)(2-naphthyl)]propyl}[(methylethyl)sulfonyl]amine (prepared in example 19).

### EXAMPLE 36

### Preparation of N-{2-[6-(1-methyl-2-{[(methylethyl)sulfonyl]amino}ethyl)-2-naphthyloxy]ethyl}acetamide

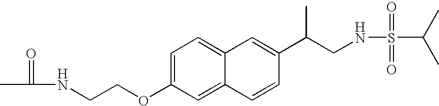

Scheme VII, step A: The title compound is prepared in a manner analogous to the procedure set forth in example 22 from {2-[6-(2-aminoethoxy)(2-naphthyl)]propyl}[(methylethyl)sulfonyl]amine (prepared in example 19).

### EXAMPLE 37

### Preparation of [(methylethyl)sulfonyl][2-(6-{2-[(phenylsulfonyl)amino]ethoxy}(2-naphthyl))propyl]amine

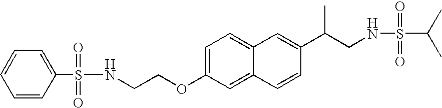

Scheme II, step D: The title compound is prepared in a manner analogous to the procedure set forth in example 23 from {2-[6-(2-aminoethoxy)(2-naphthyl)]propyl}[(methylethyl)sulfonyl]amine (prepared in example 19).

### EXAMPLE 38

### Preparation of N-{2-[6-(1-methyl-2-{[(methylethyl)sulfonyl]amino}ethyl)(2-naphthyloxy)]ethyl}benzamide

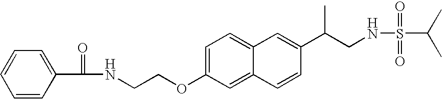

Scheme VII, step A: The title compound is prepared in a manner analogous to the procedure set forth in example 24 from {2-[6-(2-aminoethoxy)(2-naphthyl)]propyl}[(methylethyl)sulfonyl]amine (prepared in example 19).

### EXAMPLE 39

### Preparation of N-{2-[6-(1-methyl-2-{[(methylethyl)sulfonyl]amino}ethyl)-2-naphthyloxy]ethyl}butanamide

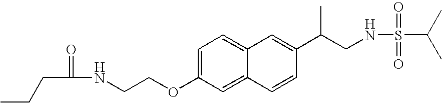

Scheme VII, step A: The title compound is prepared in a manner analogous to the procedure set forth in example 25 from {2-[6-(2-aminoethoxy)(2-naphthyl)]propyl}[(methylethyl)sulfonyl]amine (prepared in example 19)

### EXAMPLE 40

### Preparation of methoxy-N-{2-[6-(1-methyl-2-{[(methylethyl)sulfonyl]amino}ethyl)(2-naphthyloxy)]ethyl}carboxamide

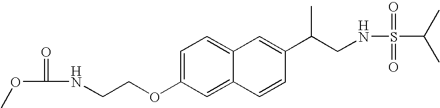

Scheme VII, step D: The title compound is prepared in a manner analogous to the procedure set forth in example 26 from {2-[6-(2-aminoethoxy)(2-naphthyl)]propyl}[(methylethyl)sulfonyl]amine (prepared in example 19)

### EXAMPLE 41

### Preparation of N-{2-[6-(1-methyl-2-{[(methylethyl)sulfonyl]amino}ethyl)(2-naphthyloxy)]ethyl}(methylphenylamino)carboxamide

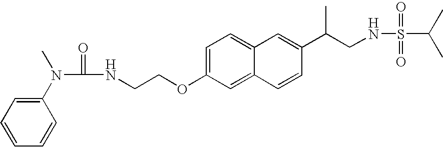

Scheme VII, step E: The title compound is prepared in a manner analogous to the procedure set forth in example 27 from {2-[6-(2-aminoethoxy)(2-naphthyl)]propyl}[(methylethyl)sulfonyl]amine (prepared in example 19)

### EXAMPLE 42

### Preparation of (methylsulfonyl)[2-(6-{2-[benzylamino]ethoxy}(2-naphthyl))ethyl]amine

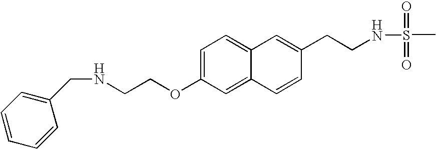

Scheme VII, step C: {2-[6-(2-Aminoethoxy)(2-naphthyl)]ethyl}(methylsulfonyl)amine (0.343 mmol, prepared in example 15) dissolved in methanol (2 mL) is added to a 15 mL round bottomed flask fitted with a stirbar and under a nitrogen atmosphere. Benzaldehyde (0.06 mL, 1.0 eq.) is treated with a catalytic amount of acetic acid and stirred at room temperature for 4 hours. Sodium borohydride (26 mg, 2.0 eq.) is added, and the reaction is stirred overnight at room temperature. The reaction mixture is then diluted with water (5 mL), and extracted with methylene chloride (3×25 mL). The organic extracts are combined and filtered through potassium carbonate, and concentrated under vacuum. The residue is further purified by silica gel chromatography, employing a Chromatotron® and a 2000 uM rotor in 100% ethyl acetate eluent to provide the title compound.

### EXAMPLE 43

### Preparation of [(methylethyl)sulfonyl][2-(6-{2-[benzylamino]ethoxy}(2-naphthyl))ethyl]amine

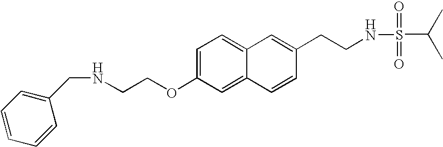

Scheme VII, step C: The title compound is prepared in a manner analogous to the procedure set forth in example 42 from {2-[6-(2-aminoethoxy)(2-naphthyl)]ethyl}[(methylethyl)sulfonyl]amine (prepared in example 16).

### EXAMPLE 44

### Preparation of [(methylethyl)sulfonyl][2-(6-{2-[benzylamino]ethoxy}(2-naphthyl))propyl]amine

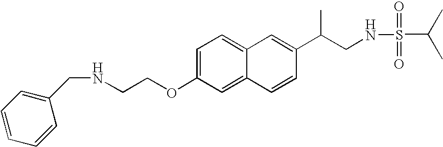

Scheme VII, step C: {2-[6-(2-Aminoethoxy)(2-naphthyl)]propyl}[(methylethyl)sulfonyl]amine (0.343 mmol, prepared in example 19) dissolved in methanol (2 mL) is added to a 15 mL round bottomed flask fitted with a stirbar and under a nitrogen atmosphere. Benzaldehyde (0.06 mL, 1.0 eq.) dissolved in acetic acid (1 mg, 0.05 eq.) is added by syringe and stirred at room temperature for 4 hours. Sodium borohydride (26 mg, 2.0 eq.) is added, and the reaction is stirred overnight at room temperature. Reaction mixture is diluted with water (5 mL), and extracted with methylene chloride (3×25 mL). The organic extracts are combined and filtered through potassium carbonate, and concentrated under vacuum, yielding 126 mg as a brown oil. This material was further purified by silica gel chromatography, employing a Chromatotron® and a 2000 uM rotor in 100% ethyl acetate eluent to provide the title compound (81 mg, 54%) as a yellow foam. Electrospray-MS 441.0 (M*+1).

### EXAMPLE 45

### Preparation of amino-N-[2-(6-{2-[(methylsulfonyl)amino]ethyl}(2-naphthyloxy))ethyl]amide

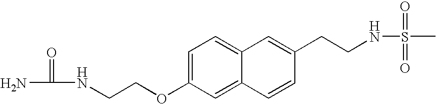

Scheme VII, step B: Trifluoroacetic acid (0.06 mL, 1.4 eq.) in toluene (1 mL) is added dropwise to a solution of {2-[6-(2-aminoethoxy)(2-naphthyl)]ethyl}(methylsulfonyl)amine (0.571 mmol, prepared in example 15) and sodium cyanate (74 mg, 2.0 eq.) in toluene (3 mL) with stirring, at 50° C., and under a nitrogen atmosphere in a 15 mL round bottomed flask. The solution is then heated to 70° C. and stirred for one hour. The reaction mixture is concentrated under vacuum. This material is washed with 1 N NaOH (15 mL), and extracted with methylene chloride (3×15 mL). The organic extracts are combined and washed with brine (1×50 mL), filtered through potassium carbonate, and concentrated under vacuum. The residue is then purified by silica gel chromatography, employing a Chromatotron® with a 4000 uM rotor and 1:1 hexanes:ethyl acetate eluent to provide the title compound.

### EXAMPLE 46

### Preparation of amino-N-{2-[6-(2-{[(methylethyl)sulfonyl]amino}ethyl)(2-naphthyloxy)]ethyl}amide

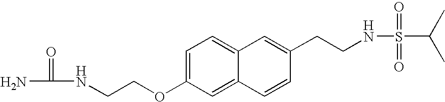

Scheme VII, step B: The title compound is prepared in a manner analogous to the procedure set forth in example 45 from {2-[6-(2-aminoethoxy)(2-naphthyl)]ethyl}[(methylethyl)sulfonyl]amine (prepared in example 16).

### EXAMPLE 47

### Preparation of amino-N-{2-[6-(1-methyl-2-{[(methylethyl)sulfonyl]amino}ethyl)(2-naphthyloxy)]ethyl}amide

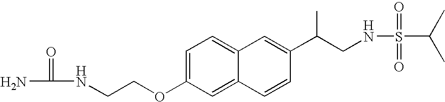

Scheme VII, step B: Trifluoroacetic acid (0.06 mL, 1.4 eq.) in toluene (1 mL) is added dropwise to {2-[6-(2-aminoethoxy)(2-naphthyl)]propyl}[(methylethyl)sulfonyl]amine (0.571 mmol, prepared in Example 19) and sodium cyanate (74 mg, 2.0 eq.) in toluene (3 mL) with stirring, at 50° C., and under a nitrogen atmosphere in a 15 mL round bottomed flask. Solution was heated to 70° C. and stirred for one hour. The reaction mixture is concentrated under vacuum. This material is washed with 1N NaOH (15 mL), and extracted with methylene chloride (3×15 mL). The organic extracts are combined and washed with brine (1×50 mL), filtered through potassium carbonate, and concentrated under vacuum to yield 400 mg orange foam. This material is purified by silica gel chromatography, employing a Chromatotron® with a 4000 uM rotor and 1:1 hexanes:ethyl acetate eluent to provide the title compound (165 mg, 73%) as a yellow liquid. Electrospray-MS 394.0 (M*+1).

### EXAMPLE 48

### Preparation of (methylamino)-N-[2-(6-{2-[(methylsulfonyl)amino]ethyl}(2-naphthyloxy))ethyl]carboxamide

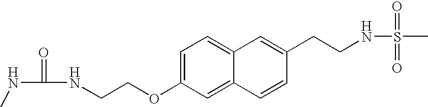

Scheme VII, step E: Methyl isocyanate (0.05 mL, 1.0 eq.) is added dropwise to {2-[6-(2-aminoethoxy)(2-naphthyl)]ethyl}(methylsulfonyl)amine (0.7133 mmol, prepared in example 15) in THF (5 mL) at 0° C., in a 15 mL round bottomed flask fitted with a stirbar. The reaction is stirred overnight in an ice bath, gradually warming to room temperature. The reaction mixture is then quenched with water (5 mL) and is extracted with methylene chloride (3×15 mL). The organic extracts are combined and washed with brine (1×50 mL), filtered through MgSO₄, and concentrated under vacuum. This material is then purified by silica gel chromatography, employing a Chromatotron® with a 2000 uM rotor and 1:1 methylene chloride:ethyl acetate eluent to provide the title compound.

### EXAMPLE 49

### Preparation of (methylamino)-N-{2-[6-(2-{[(methylethyl)sulfonyl]amino}ethyl)(2-naphthyloxy)]ethyl}carboxamide

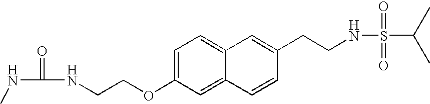

Scheme VII, step E: The title compound is prepared in a manner analogous to the procedure set forth in example 48 from {2-[6-(2-aminoethoxy)(2-naphthyl)]ethyl}[(methylethyl)sulfonyl]amine (prepared in example 16).

### EXAMPLE 50

### Preparation of (methylamino)-N-{2-[6-(1-methyl-2-{[(methylethyl)sulfonyl]amino}ethyl)(2-naphthyloxy)]ethyl}carboxamide

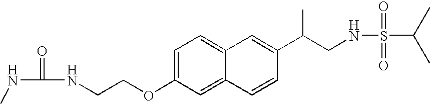

Scheme VII, step E: Methyl isocyanate (0.05 mL, 1.0 eq.) is added dropwise to {2-[6-(2-aminoethoxy)(2-naphthyl)]propyl}[(methylethyl)sulfonyl]amine (0.7133 mmol, prepared in Example 19) in THF (5 mL) at 0° C., in a 15 mL round bottomed flask fitted with a stirbar. The reaction is then stirred overnight in ice bath, gradually warming to room temperature. The reaction mixture is quenched with water (5 mL) and is extracted with methylene chloride (3×15 mL). The organic extracts are combined and washed with brine (1×50 mL), filtered through MgSO₄, and concentrated under vacuum, yielding 184 mg yellow foam. This material is purified by silica gel chromatography, employing a Chromatotron® with a 2000 uM rotor and 1:1 methylene chloride:ethyl acetate eluent to provide the title compound (130 mg, 45%) as a colorless oil.

Electrospray-MS 408.0 (M*+1).

### EXAMPLE 51

### Preparation of (2-{6-[2-(dimethylamino)ethoxy](2-naphthyl)}ethyl)(methylsulfonyl)amine

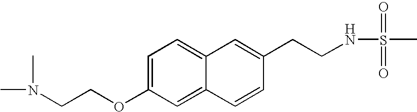

Scheme VII, step C:. To a stirring solution of {2-[6-(2-aminoethoxy)(2-naphthyl)]ethyl}(methylsulfonyl)amine (0.713 mmol, prepared in example 15 in methanol (5 mL) is added formaldehyde (0.85 mL, 16 eq.) over a 15 minute period and let the mixture to stirr at room temperature for one hour. Sodium borohydride (216 mg, 8 eq) is added to the reaction and the mixture is stirred at room temperature over night. The reaction is concentrated under vacuum and the crude product is dissolved in 2:1 methylene chloride:water (30 mL). Organic material is extracted with methylene chloride (2×30 mL) and the combined organic layer is washed with water (30 mL) and brine (30 mL), dried over MgSO₄, and concentrated under vacuum. The residue is then purified by silica gel chromatography, employing a Chromatotron® with a 2000 uM rotor and 9:1 methylene chloride:methanol eluent to provide the title compound.

### EXAMPLE 52

### Preparation of (2-{6-[2-(dimethylamino)ethoxy](2-naphthyl)}ethyl)[(methylethyl)sulfonyl]amine

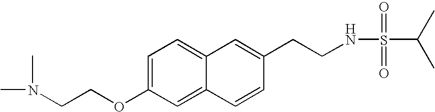

Scheme VII, step C: The title compound is prepared in a manner analogous to the procedure set forth in example 51 from {2-[6-(2-aminoethoxy)(2-naphthyl)]ethyl}[(methylethyl)sulfonyl]amine (prepared in example 16).

### EXAMPLE 53

### Preparation of (2-{6-[2-(dimethylamino)ethoxy](2-naphthyl)}propyl)[(methylethyl)sulfonyl]amine

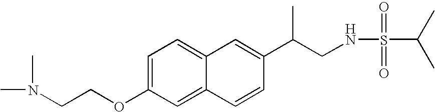

Scheme VII, step C: To a stirring solution of {2-[6-(2-aminoethoxy)(2-naphthyl)]propyl}[(methylethyl)sulfonyl]amine (0.713 mmol, prepared in Example 19) in methanol (5 mL) is added formaldehyde (0.85 mL, 16 eq.) over a 15 minute period and let the mixture to stirr at room temperature for one hour. Sodium borohydride (216 mg, 8 eq) is added to the reaction and the mixture is stirred at room temperature over night. The reaction is concentrated under vacuum and the crude product is dissolved in 2:1 methylene chloride:water (30 mL). Organic material is extracted with methylene chloride (2×30 mL) and the combined organic layer is washed with water (30 mL) and brine (30 mL), dried over MgSO₄, and concentrated under vacuum. The residue is then purified by silica gel chromatography, employing a Chromatotron® with a 2000 uM rotor and 9:1 methylene chloride:methanol eluent to provide the title compound (42 mg, 16%) as a yellow foam. Electrospray-MS 379.0 (M*+1).

Elemental Analysis:

| | | | |
|---|---|---|---|
| C(Theory): | 63.46 | C(Found): | 62.15 |
| H(Theory): | 7.99 | H(Found): | 7.49 |
| N(Theory): | 7.40 | N(Found): | 7.10 |

The ability of compounds of formula I to potentiate glutamate receptor-mediated response may be determined using fluorescent calcium indicator dyes (Molecular Probes, Eugene, Oreg., Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 mL of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl₂, 2 CaCl₂, 10 glucose, pH=7.4 with NaOH, 295 mOsm kg-1. The intracellular recording solution contains (in mM): 140 CsCl, 1 MgCl₂, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg-1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

According to another aspect, the present invention provides a pharmaceutical composition, which comprises a compound of formula I or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 1 mg to about 500 mg, more preferably about 5 mg to about 300 mg (for example 25 mg) of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

As used herein the term “patient” refers to a mammal, such as a mouse, guinea pig, rat, dog or human. It is understood that the preferred patient is a human.

As used herein, the terms “treating” or “to treat” each mean to alleviate symptoms, eliminate the causation either on a temporary or permanent basis, or to prevent or slow the appearance of symptoms of the named disorder. As such, the methods of this invention encompass both therapeutic and prophylactic administration.

As used herein, the term “effective amount” refers to the amount of a compound of formula I which is effective, upon single or multiple dose administration to a patient, in treating the patient suffering from the named disorder.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of formula I can be administered by a variety of routes including oral, rectal, transdermal, subcutaneous, intravenous, intramuscular, bucal or intranasal routes. Alternatively, the compounds of formula I may be administered by continuous infusion. A typical daily dose will contain from about 0.01 mg/kg to about 100 mg/kg of the compound of formula I. Preferably, daily doses will be about 0.05 mg/kg to about 50 mg/kg, more preferably from about 0.1 mg/kg to about 25 mg/kg.

The compounds of the present invention as a class are particularly useful in the treatment methods of the present invention, but certain groups, substituents, and configurations are preferred. The following paragraphs describe such preferred groups, substituents, and configurations. It will be understood that these preferences are applicable both to the treatment methods and to the new compounds of the present invention.

a) R is preferably hydrogen;

b) W is preferably R⁸SO₂— or R¹³C(═O), and most preferably R⁸SO₂.

c) R¹ is preferably (1-4C)alkyl, CF₃, N(CH₃)₂, or NH(CH₃), most preferably methyl, ethyl, propyl, CF₃, or 2-propyl, and it is most especially preferred that R¹ is 2-propyl;

d) R² is preferably hydrogen, F, methyl, ethyl, propyl, hydroxy, or methoxy, most preferably hydrogen, F, hydroxy, or methyl, and it is most especially preferred that R² is F or methyl;

e) R^{3a} is preferably hydrogen, F, methyl, ethyl, propyl, hydroxy, or methoxy, and most preferably hydrogen, F, hydroxy, or methyl;

f) R^{3b} is preferably hydrogen, methyl, ethyl, propyl, or methoxy, most preferably hydrogen, or methyl, and it is most especially preferred that R^{3b} is hydrogen;

g) R^{4a} is preferably hydrogen, F, methyl, ethyl, methoxy, or ethoxy, and most preferably hydrogen, F, methyl or methoxy, and it is most especially preferred that R^{4a} is hydrogen;

h) R^{4b} is preferably hydrogen, F, methyl, ethyl, methoxy, or ethoxy, and most preferably hydrogen, F, methyl or methoxy, and it is most especially preferred that R^{4b} is hydrogen;

i) R⁵ is preferably hydrogen, methyl, or ethyl, most preferably hydrogen or methyl, and it is most especially preferred that R⁵ is hydrogen;

j) R⁶ is preferably hydrogen, methyl, or ethyl, most preferably hydrogen or methyl, and it is most especially preferred that R⁶ is hydrogen;

k) R⁷ is preferably hydrogen or methyl with hydrogen being most preferred;

l) R⁸ is preferably (1-4C)alkyl, CF₃, N(CH₃)₂, phenyl, or NH(CH₃), most preferably methyl, ethyl, propyl, 2-propyl, phenyl, or CF₃, and it is most especially preferred that R⁸ is 2-propyl;

m) R⁹ is preferably hydrogen or methyl;

n) R¹⁰ is preferably hydrogen or methyl;

o) R¹¹ is preferably hydrogen, methyl or ethyl, and most preferably hydrogen;

p) n is preferably zero, 1, 2 or 3, most preferably zero or 1, and it is most especially preferred that n is zero;

q) m is preferably 1, 2 or 3, and most preferably 1 or 2;

r) p is preferably 1;

s) When R² is hydrogen, R^{3a} is preferably F or methyl;

t) When R^{3a} is hydrogen, R² is preferably F or methyl;

u) R¹² is preferably methyl;

v) R¹³ is preferably methyl, ethyl, propyl, or phenyl;

w) R¹⁴ is preferably methyl, ethyl, or propyl, with methyl being most preferred;

x) R¹⁵ is preferably methyl, ethyl, or propyl, with methyl being most preferred;

y) R¹⁶ is preferably methyl, ethyl, propyl, or benzyl, with methyl and benzyl being most preferred;

z) B is preferably;

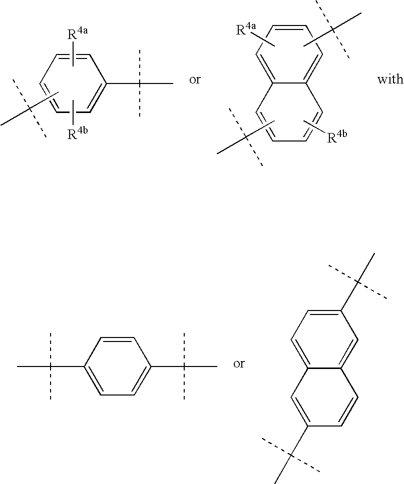

being most preferred.

aa) In addition, when W is R¹³C(═O)— or R¹⁶, it is especially preferred that B is

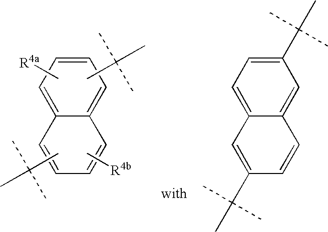

being most especially preferred.

We claim:

1. A compound of the formula: 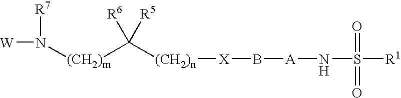
wherein
A represents 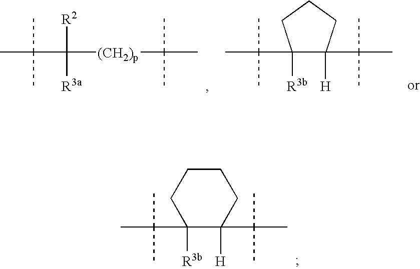
B represents 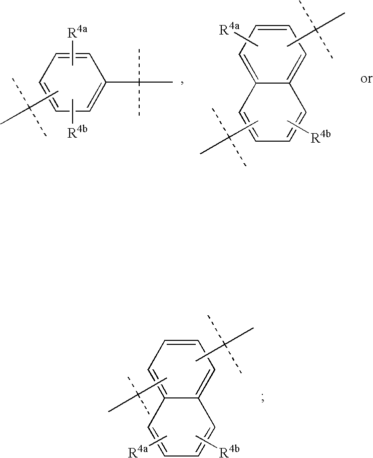
X represents O, NR, or S:
W represents R⁸SO₂—, R¹³C(═O)—, R¹³R¹⁵NC(═O)—, H₂NC(═O)—, R¹⁶, or
R¹⁴OC(═O)—;
R represents hydrogen, (1-6C)alkyl, or -(1-4C)alkylaromatic;
R¹ represents (1-6C)alkyl, (2-6C)alkenyl, halo(1-4C)alkyl, or NR⁹R¹⁰;
R² and R^{3a} each independently represent hydrogen, (1-4C)alkyl, F, or —OR¹¹;
R^{3b} represents hydrogen, (1-4C)alkyl, or —OR¹²;
R^{4a} and R^{4b} each independently represent hydrogen, (1-4C) alkyl, (1-4C)alkoxy, I, Br, Cl, or F;
R⁵ and R⁶ each independently represent hydrogen, (1-4C)alkyl, F, or —OR¹¹;
R⁷ represents hydrogen, (1-4C)alkyl or -(1-4C)alkylaromatic;
R⁸ represents (1-6C)alkyl,-(1-4C)alkylphenyl, halo(1-4C)alkyl, unsubstituted or substituted aromatic group, unsubstituted or substituted heteroaromatic group, cycloalkyl, alkylcycloalkyl, or NR⁹R¹⁰;
n is zero or an integer 1, 2, 3, 4, or 5;
m is zero or an integer 1, 2, 3, 4, or 5;
p is an integer 1 or 2;
R⁹ and R¹⁰ each independently represent hydrogen or (1-4C)alkyl;
R¹¹ represents hydrogen or (1-4C)alkyl;
R¹² represents (1-4C)alkyl;
R¹³ represents phenyl or (1-6C)alkyl;
R¹⁴ represents (1-6C)alkyl;
R¹⁵ represents (1-4C)alkyl; and
R¹⁶ represents (1-4C)alkyl or -(1-4C)alkylphenyl;
or a pharmaceutically acceptable salt thereof, with the proviso that when W is R¹⁶, then B is other than

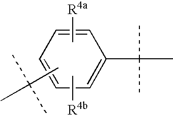

2. A compound according to claim 1 wherein R¹ is (1-6C)alkyl.

3. A compound according to claim 2 wherein R¹ is methyl, ethyl, or 2-propyl.

4. A compound according to claim 3 wherein R¹ is 2-propyl.

5. A compound according to any one of claims 1 to 4 wherein R⁷ is hydrogen.

6. A compound according to any one of claims 1 to 5 wherein X is NR.

7. A compound according to any one of claims 1 to 5 wherein X is O.

8. A compound according to any one of claims 1 to 5 wherein X is S.

9. A compound according to claim 6 wherein R is hydrogen.

10. A compound according to any one of claims 1 to 9 wherein B represents:

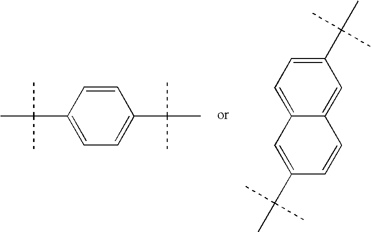

11. A compound according to any one of claims 1 to 9 wherein B represents:

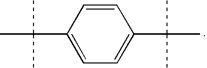

12. A compound according to any one of claims 1 to 9 wherein R^{4a} and R^{4b} each represent hydrogen.

13. A compound according to any one of claims 1 to 12 wherein R² represents methyl and R^{3a} represents hydrogen.

14. A compound according to any one of claims 1 to 12 wherein R² represents methyl and R^{3a} represents hydroxy.

15. A compound according to any one of claims 1 to 14 wherein P represents 1.

16. A compound according to any one of claims 1 to 15 wherein R^{3b} represents hydrogen.

17. A compound according to any one of claims 1 to 16 wherein R⁵ represents hydrogen and R⁶ represent hydrogen or methyl.

18. A compound according to claim 17 wherein R⁶ represents hydrogen.

19. A compound according to any one of claims 1 to 18 wherein n represents zero.

20. A compound according to any one of claims 1 to 19 wherein m represents 1, 2 or 3.

21. A compound according to any one of claims 1 to 20 wherein W represents R⁸SO₂—;

22. A compound according to claim 21 wherein R⁸ represents methyl, ethyl, propyl, 2-propyl, trifluoromethyl or phenyl.

23. A compound according to claim 22 wherein R⁸ represents methyl or 2-propyl.

24. A pharmaceutical composition, which comprises a compound as claimed in any one of claims 1 to 23 and a pharmaceutically acceptable diluent or carrier.

25. A method of potentiating glutamate receptor function in a patient, which comprises administering to said patient an effective amount of a compound according to claim 1.

26. A method of treating a cognitive disorder; Alzheimer's disease; age-related dementia; age-induced memory impairment; depression; attention deficit disorder; attention deficit hyperactivity disorder; psychosis; cognitive deficits associated with psychosis; drug-induced psychosis, Parkinson's disease, or stroke in a patient, which comprises administering to a patient an effective amount of a compound according to claim 1.

27. A compound according to any of claims 1 to 23, or a pharmaceutically acceptable salt thereof, for use as a pharmaceutical.

28. The use of a compound according to any of claims 1 to 23, or a pharmaceutically acceptable salt thereof, for the manufacture of a medicament for potentiating glutamate receptor function.

29. The use of a compound according to any of claims 1 to 23 for the manufacture of a medicament for treating a cognitive disorder; Alzheimer's disease; age-related dementia; age-induced memory impairment; depression; attention deficit disorder; attention deficit hyperactivity disorder; psychosis; cognitive deficits associated with psychosis; drug-induced psychosis, Parkinson's disease, or stroke.
